(19) United States
(12) Patent Application Publication
(10) Pub. No.: US 2016/0293850 A1
(43) Pub. Date: Oct. 6, 2016

(54) CONDENSED CYCLIC COMPOUND AND ORGANIC LIGHT-EMITTING DEVICE INCLUDING THE SAME

(71) Applicant: Samsung Display Co., Ltd., Yongin-si (KR)

(72) Inventor: Jungsub Lee, Yongin-si (KR)

(21) Appl. No.: 15/065,733

(22) Filed: Mar. 9, 2016

(30) Foreign Application Priority Data
Mar. 31, 2015 (KR) 10-2015-0045333

(51) Int. Cl. H01L 51/00; C09K 11/06; C07D 493/04; C07D 495/04; C09K 11/02

(52) U.S. Cl. CPC H01L 51/0071; C07D 495/04; C09K 11/025; C07D 493/04; H01L 51/0085; C09K 11/06; H01L 51/5012

(57) ABSTRACT

An organic light-emitting device includes: a first electrode; a second electrode facing the first electrode; and an organic layer between the first electrode and the second electrode, the organic layer including an emission layer and at least one condensed cyclic compound of Formula 1. According to one or more embodiments of the present disclosure, an organic light-emitting device including the condensed cyclic compound may have a low driving voltage, a high efficiency, a high luminance, and a long lifespan.

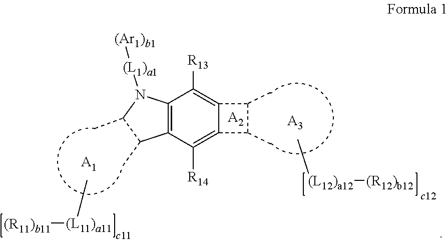

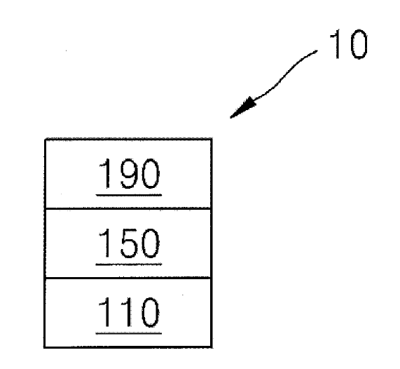

### CROSS-REFERENCE TO RELATED APPLICATION

This application claims priority to and the benefit of Korean Patent Application No. 10-2015-0045333, filed on Mar. 31, 2015, in the Korean Intellectual Property Office, the entire content of which is incorporated herein by reference.

### BACKGROUND

1. Field

One or more aspects of example embodiments relate to a condensed cyclic compound and an organic light-emitting device including the same.

2. Description of the Related Art

Organic light-emitting devices are self-emission devices that have a wide viewing angle, a high contrast ratio, a fast response rate, and excellent brightness, driving voltage, and response speed characteristics, and can produce full-color images.

The organic light-emitting device may have a structure in which a first electrode is positioned on a substrate, and a hole transport region, an emission layer, an electron transport region, and a second electrode are sequentially formed on the first electrode. Holes provided from the first electrode may move toward the emission layer through the hole transport region, and electrons provided from the second electrode may move toward the emission layer through the electron transport region. Carriers such as the holes and the electrons are then recombined in the emission layer to produce excitons. These excitons change from an excited state to a ground state, thereby generating light.

### SUMMARY

One or more aspects of example embodiments are directed toward a condensed cyclic compound and an organic light-emitting device including the same.

Additional aspects will be set forth in part in the description which follows and, in part, will be apparent from the description, or may be learned by practice of the presented embodiments.

According to one or more example embodiments, a condensed cyclic compound is represented by Formula 1:

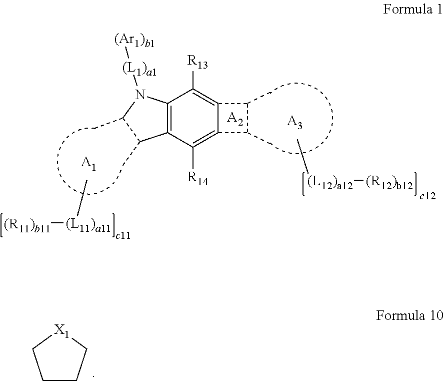

In Formulae 1 and 10,

A₂ ring and A₃ ring may be fused to each other,

A₁ ring and A₃ ring may be each independently a naphthalene ring,

A₂ ring may be represented by Formula 10, and X₁ in Formula 10 may be an oxygen atom (O) or a sulfur atom (S),

L₁, L₁₁, and L₁₂ may be each independently selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

a1, a11, and a12 may be each independently selected from 0, 1, 2, and 3, when a1 is 2 or more, two or more L₁(s) may be identical to or different from each other, when a11 is 2 or more, two or more L₁₁(s) may be identical to or different from each other, and when a12 is 2 or more, two or more L₁₂(s) may be identical to or different from each other,

Ar₁ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group,

b1 may be selected from 1, 2, and 3, and when b1 is 2 or more, two or more Ar₁(s) may be identical to or different from each other,

R₁₁ to R₁₄ may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), and N(Q₆)(Q₇),

b11 and b12 may be each independently selected from 0, 1, 2, and 3, when b11 is 2 or more, two or more R₁₁(s) may be identical to or different from each other, and when b12 is 2 or more, two or more R₁₂(s) may be identical to or different from each other,

c11 and c12 may be each independently selected from 0, 1, 2, 3, 4, 5, and 6, when c11 is 2 or more, two or more *-[(L₁₁)ₐ₁₁-(R₁₁)_{b11}](s) may be identical to or different from each other, and when c12 is 2 or more, two or more *-[(L₁₂)ₐ₁₂-(R₁₂)_{b12}](s) may be identical to or different from each other, and

at least one of substituents of the substituted C₃-C₁₀ cycloalkylene group, substituted C₁-C₁₀ heterocycloalkylene group, substituted C₃-C₁₀ cycloalkenylene group, substituted C₁-C₁₀ heterocycloalkenylene group, substituted C₆-C₆₀ arylene group, substituted C₁-C₆₀ heteroarylene group, substituted divalent non-aromatic condensed polycyclic group, substituted divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₁-C₆₀ heteroaryl group, substituted monovalent non-aromatic condensed polycyclic group, and substituted monovalent non-aromatic condensed heteropolycyclic group may be selected from:

deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —B(Q₁₄)(Q₁₅), and —N(Q₁₆)(Q₁₇);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —B(Q₂₄)(Q₂₅), and —N(Q₂₆)(Q₂₇); and

—Si(Q₃₁)(Q₃₂)(Q₃₃), —B(Q₃₄)(Q₃₅), and —N(Q₃₆)(Q₃₇),

wherein Q₁ to Q₇, Q₁₁ to Q₁₇, Q₂₁ to Q₂₇, and Q₃₁ to Q₃₇ may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

According to one or more example embodiments of the present disclosure, an organic light-emitting device includes a first electrode, a second electrode facing the first electrode, and an organic layer between the first electrode and the second electrode, the organic layer including an emission layer, wherein the organic layer further includes at least one selected from condensed cyclic compound of Formula 1.

### BRIEF DESCRIPTION OF THE DRAWINGS

These and/or other aspects will become apparent and more readily appreciated from the following description of the example embodiments, taken in conjunction with the drawing, which is a schematic view of a structure of an organic light-emitting device according to one or more embodiments of the present disclosure.

### DETAILED DESCRIPTION

Reference will now be made in detail to example embodiments, an example of which is illustrated in the accompanying drawing. In this regard, the present example embodiments may have different forms and should not be construed as being limited to the descriptions set forth herein. Accordingly, the example embodiments are merely described below, by referring to the drawing, to explain aspects of the present description. As used herein, the term “and/or” includes any and all combinations of one or more of the associated listed items. Expressions such as “at least one selected from,” “one selected from,” and “one of,” when preceding a list of elements, modify the entire list of elements and do not modify the individual elements of the list. Further, the use of “may” when describing embodiments of the present invention refers to “one or more embodiments of the present invention.”

A condensed cyclic compound according to an embodiment of the present disclosure is represented by Formula 1:

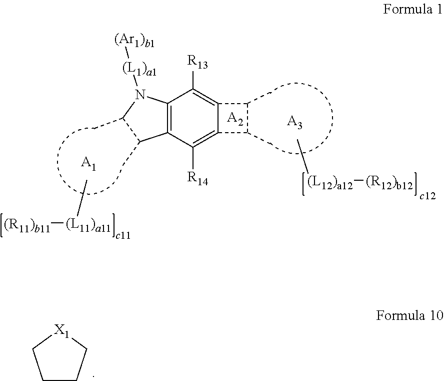

In Formulae 1 and 10,

A₂ ring and A₃ ring may be condensed to each other,

A₁ ring and A₃ ring may be each independently a naphthalene ring, and

A₂ may be represented by Formula 10, where X₁ in Formula 10 is O (an oxygen atom) or S (a sulfur atom).

L₁, L₁₁, and L₁₂ in Formula 1 may be each independently selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group.

For example, L₁, L₁₁, and L₁₂ in Formula 1 may be each independently selected from:

a phenylene group, a pentalenylene group, an indenylene group, a naphthylene group, an azulenylene group, a heptalenylene group, an indacenylene group, an acenaphthylene group, a fluorenylene group, a spiro-fluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a pyrrolylene group, a thiophenylene group, a furanylene group, an imidazolylene group, a pyrazolylene group, a thiazolylene group, an isothiazolylene group, an oxazolylene group, an isoxazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzoimidazolylene group, a benzofuranylene group, a benzothiophenylene group, an isobenzothiazolylene group, a benzoxazolylene group, an isobenzoxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, a thiadiazolylene group, an imidazopyridinylene group, and an imidazopyrimidinylene group; and

a phenylene group, a pentalenylene group, an indenylene group, a naphthylene group, an azulenylene group, a heptalenylene group, an indacenylene group, an acenaphthylene group, a fluorenylene group, a spiro-fluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a pyrrolylene group, a thiophenylene group, a furanylene group, an imidazolylene group, a pyrazolylene group, a thiazolylene group, an isothiazolylene group, an oxazolylene group, an isoxazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzoimidazolylene group, a benzofuranylene group, a benzothiophenylene group, an isobenzothiazolylene group, a benzoxazolylene group, an isobenzoxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, a thiadiazolylene group, an imidazopyridinylene group, and an imidazopyrimidinylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group.

In some embodiments, L₁, L₁₁, and L₁₂ in Formula 1 may be each independently selected from groups represented by Formulae 3-1 to 3-38:

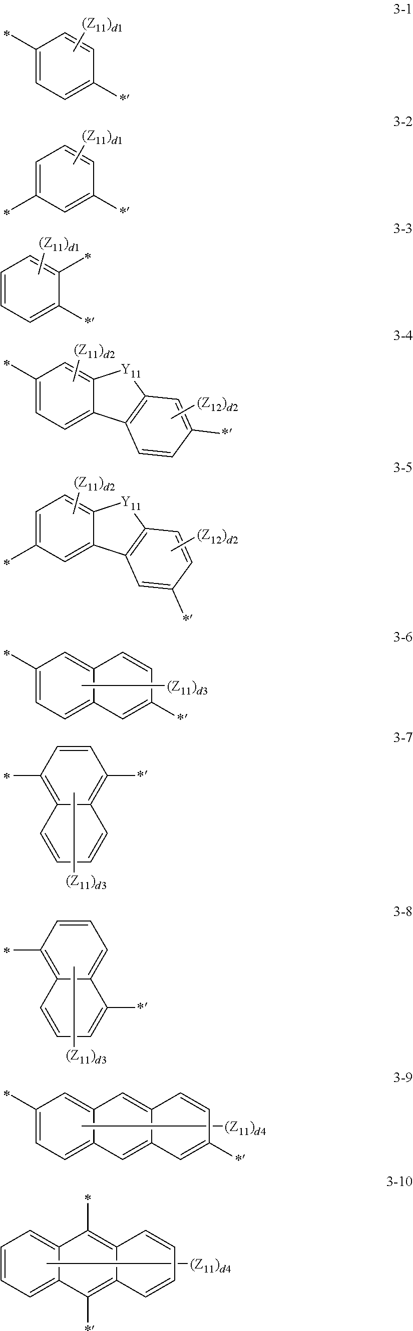

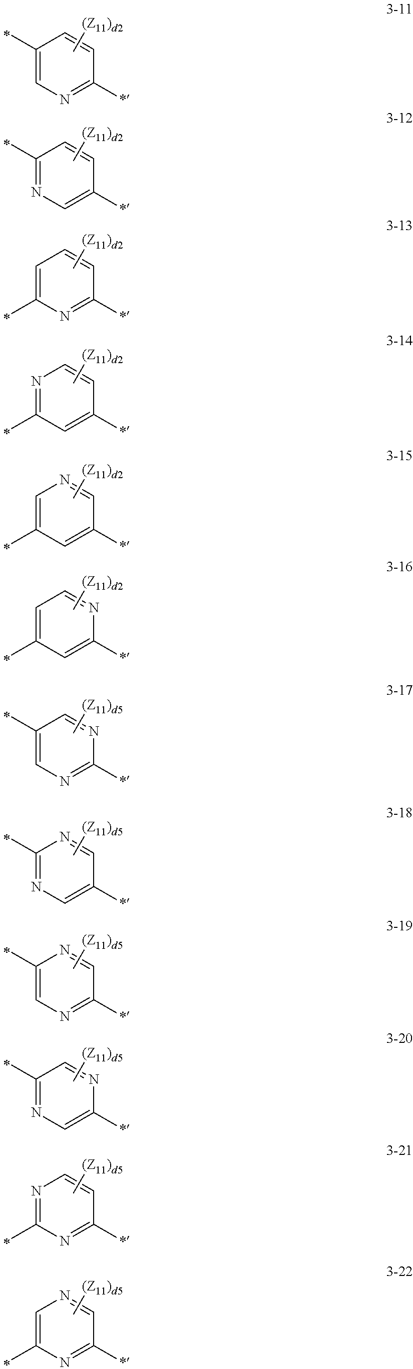

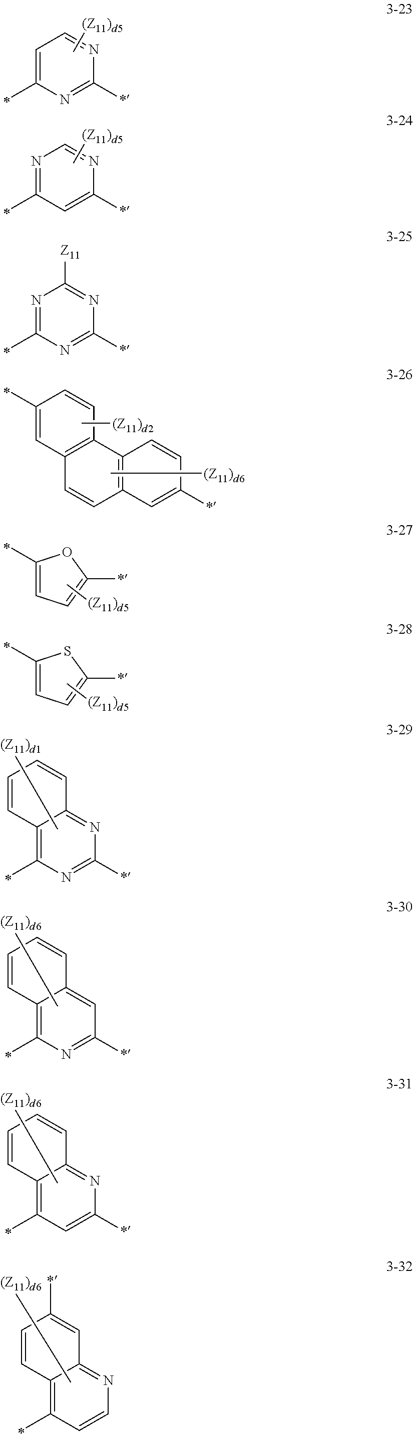

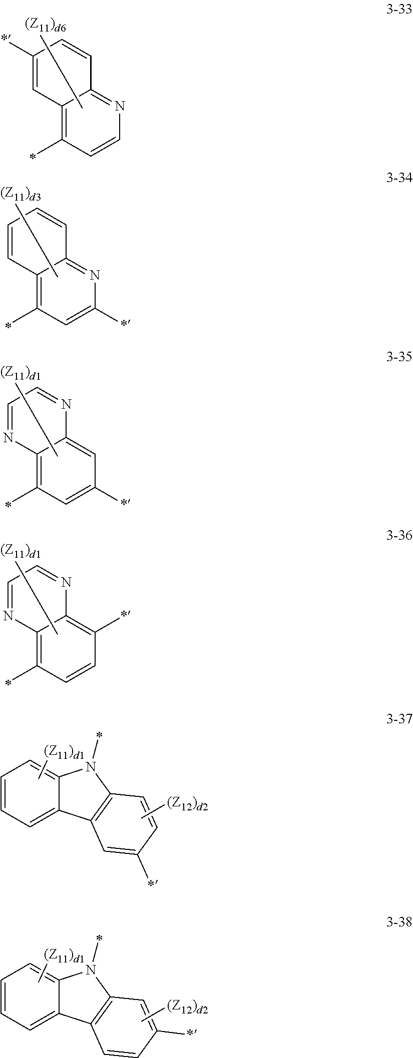

In Formulae 3-1 to 3-38,

Y₁₁ may be selected from O, S, C(Z₁₃)(Z₁₄), N(Z₁₅), and Si(Z₁₆)(Z₁₇),

Z₁₁ to Z₁₇ may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

d1 may be an integer selected from 1, 2, 3, and 4, d2 may be an integer selected from 1, 2, and 3, d3 may be an integer selected from 1, 2, 3, 4, 5, and 6, d4 may be an integer selected from 1, 2, 3, 4, 5, 6, 7, and 8, d5 may be 1 or 2, d6 may be an integer selected from 1, 2, 3, 4, and 5, and each of * and *′ indicates a binding site to a neighboring atom.

In some embodiments, L₁, L₁₁, and L₁₂ in Formula 1 may be each independently selected from:

a phenylene group, a naphthylene group, a pyridinylene group, a dibenzofuranylene group, and a dibenzothiophenylene group; and

a phenylene group, a naphthylene group, a pyridinylene group, a dibenzofuranylene group, and a dibenzothiophenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a phenyl group, a naphthyl group, a pyridinyl group, a pyrimidinyl group, and a triazinyl group, but are not limited thereto.

In some embodiments, L₁, L₁₁, and L₁₂ in Formula 1 may be each independently selected from groups represented by Formulae 4-1 to 4-25, but are not limited thereto:

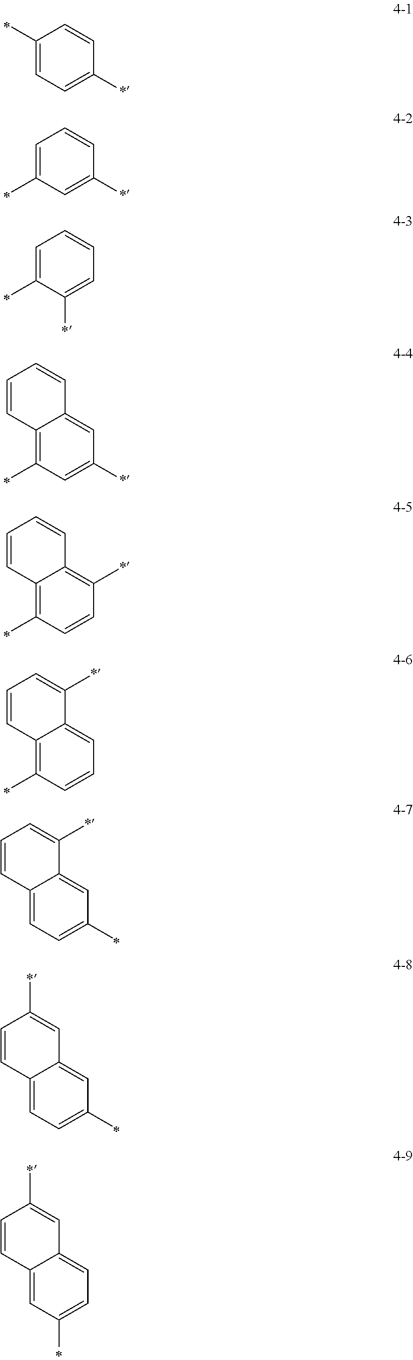

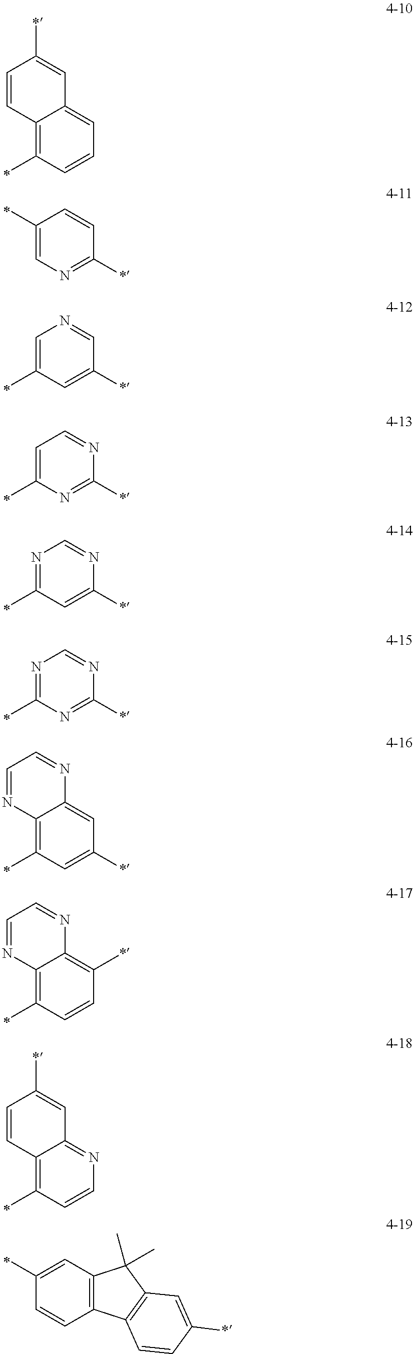

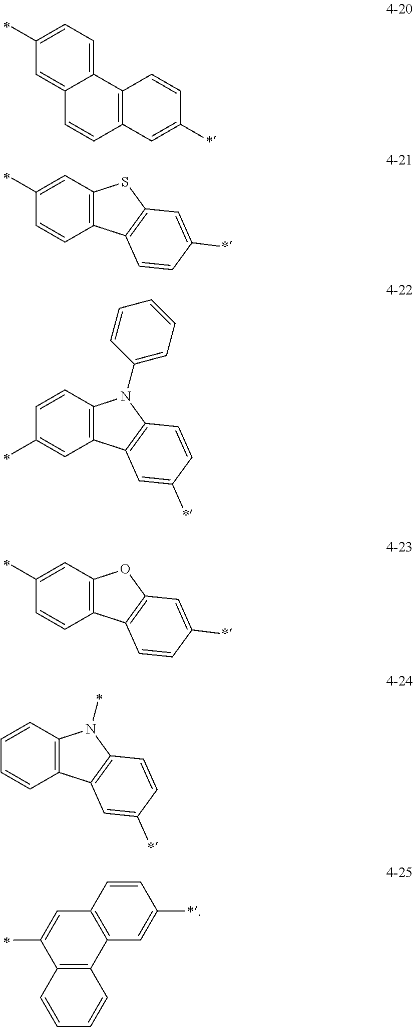

Each of * and *′ in Formulae 4-1 to 4-25 indicates a binding site to a neighboring atom.

In Formula 1, a1, a11, and a12 may be each independently selected from 0, 1, 2, and 3. a1 indicates the number of L₁ in Formula 1, and when a1 is 2 or more, two or more L₁(s) may be identical to or different from each other. When a1 is 0, -(L₁)ₐ₁- is a single bond. In some embodiments, a11 may be 0, 1, or 2. a11 indicates the number of L₁₁ in Formula 1, and when a11 is 2 or more, two or more L₁₁(s) may be identical to or different from each other. When a11 is 0, -(L₁₁)ₐ₁₁- is a single bond. In some embodiments, a12 may be 0, 1, or 2. When a12 is 0, -(L₁₂)ₐ₁₂- is a single bond. a12 indicates the number of L₁₂ in Formula 1, and when a12 is 2 or more, two or more L₁₂(s) may be identical to or different from each other. In some embodiments, a12 may be 0 or 1. For example, a11 and a12 in Formula 1 may be both 0.

Ar₁ in Formula 1 may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

For example, Ar₁ in Formula 1 may be selected from:

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a dibenzosilolyl group, a thiadiazolyl group, an imidazopyridinyl group, and an imidazopyrimidinyl group; and

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a dibenzosilolyl group, a thiadiazolyl group, an imidazopyridinyl group, and an imidazopyrimidinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, and —Si(Q₃₁)(Q₃₂)(Q₃₃),

where Q₃₁ to Q₃₃ may be each independently selected from a C₁-C₁₀ alkyl group, a C₁-C₁₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a carbazolyl group, a phenanthrolinyl group, a benzoimidazolyl group, a benzofuranyl group, a benzothiophenyl group, an isobenzothiazolyl group, a benzoxazolyl group, an isobenzoxazolyl group, a triazolyl group, an oxadiazolyl group, a triazinyl group, a dibenzofuranyl group, a dibenzothiophenyl group, a benzocarbazolyl group, a dibenzocarbazolyl group, a dibenzosilolyl group, a thiadiazolyl group, an imidazopyridinyl group, and an imidazopyrimidinyl group.

In some embodiments, Ar₁ in Formula 1 may be selected from:

a phenyl group, a naphthyl group, a fluorenyl group, a spiro-fluorenyl group, a benzofluorenyl group, a dibenzofluorenyl group, a phenanthrenyl group, an anthracenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a carbazolyl group, a benzoimidazolyl group, a benzofuranyl group, a benzothiophenyl group, an isobenzothiazolyl group, a benzoxazolyl group, an isobenzoxazolyl group, an oxadiazolyl group, a triazinyl group, a dibenzofuranyl group, a dibenzothiophenyl group, an imidazopyridinyl group, and an imidazopyrimidinyl group; and

a phenyl group, a naphthyl group, a fluorenyl group, a spiro-fluorenyl group, a benzofluorenyl group, a dibenzofluorenyl group, a phenanthrenyl group, an anthracenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a carbazolyl group, a benzoimidazolyl group, a benzofuranyl group, a benzothiophenyl group, an isobenzothiazolyl group, a benzoxazolyl group, an isobenzoxazolyl group, an oxadiazolyl group, a triazinyl group, a dibenzofuranyl group, a dibenzothiophenyl group, an imidazopyridinyl group, and an imidazopyrimidinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a carbazolyl group, a benzoimidazolyl group, a benzofuranyl group, a benzothiophenyl group, an isobenzothiazolyl group, a benzoxazolyl group, an isobenzoxazolyl group, an oxadiazolyl group, a triazinyl group, a dibenzofuranyl group, a dibenzothiophenyl group, an imidazopyridinyl group, an imidazopyrimidinyl group, and —Si(Q₃₁)(Q₃₂)(Q₃₃),

where Q₃₁ to Q₃₃ may be each independently selected from a C₁-C₁₀ alkyl group, a C₁-C₁₀ alkoxy group, a phenyl group, and a naphthyl group. However, embodiments of the present disclosure are not limited thereto.

In some embodiments, Ar₁ in Formula 1 may be selected from:

a phenyl group, a naphthyl group, a fluorenyl group, a phenanthrenyl group, a pyridinyl group, a pyrimidinyl group, a triazinyl group, a dibenzofuranyl group, and a dibenzothiophenyl group; and

a phenyl group, a naphthyl group, a fluorenyl group, a phenanthrenyl group, a pyridinyl group, a pyrimidinyl group, a triazinyl group, a dibenzofuranyl group, and a dibenzothiophenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a fluorenyl group, a phenanthrenyl group, a pyridinyl group, a pyrimidinyl group, a triazinyl group, a dibenzofuranyl group, a dibenzothiophenyl group, and —Si(Q₃₁)(Q₃₂)(Q₃₃),

where Q₃₁ to Q₃₃ may be each independently selected from a C₁-C₁₀ alkyl group, a C₁-C₁₀ alkoxy group, a phenyl group, and a naphthyl group.

In some embodiments, Ar₁ may be selected from groups represented by Formulae 5-1 to 5-42, but is not limited thereto:

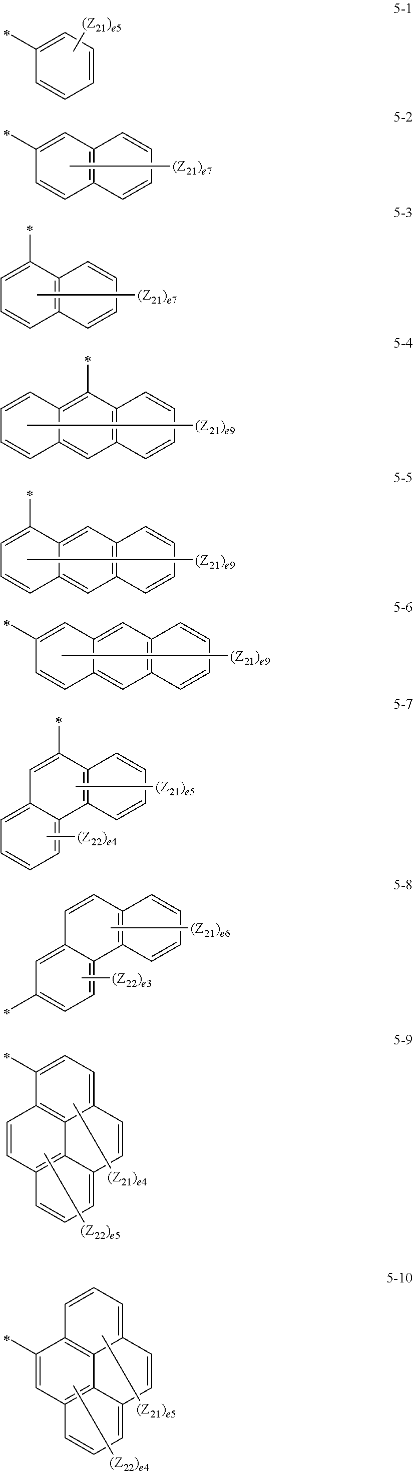

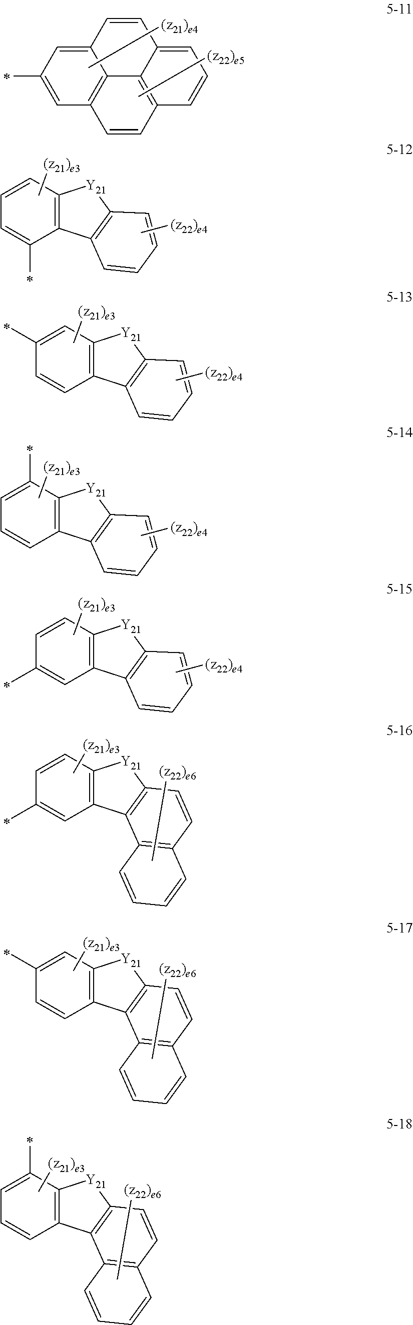

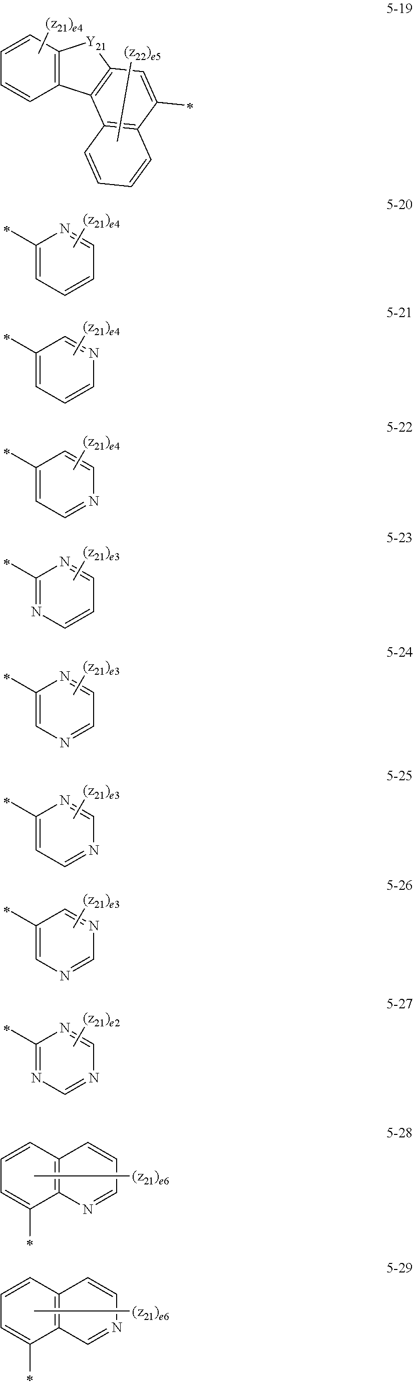

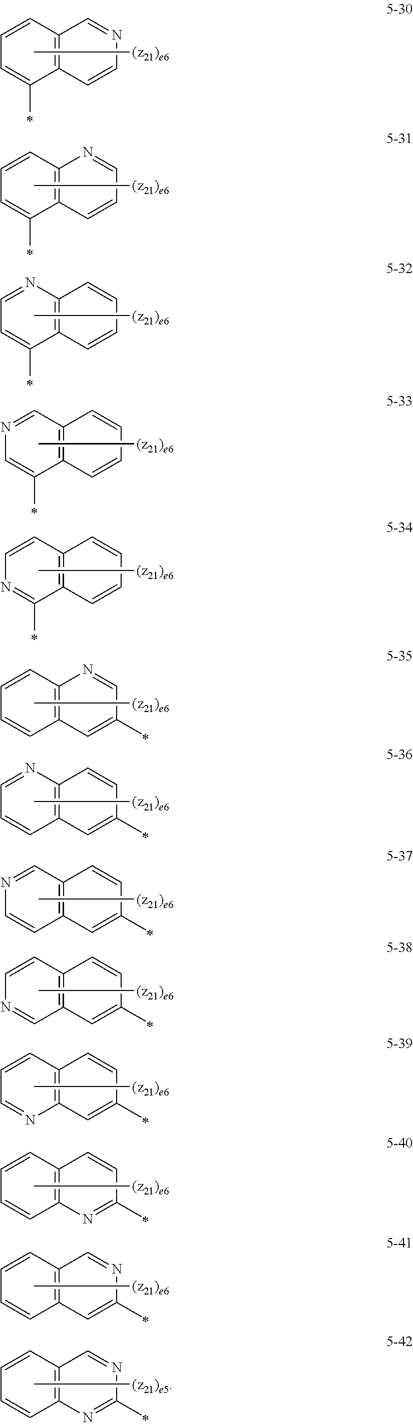

In Formulae 5-1 to 5-42,

Y₂₁ may be selected from O, S, C(Z₂₃)(Z₂₄), N(Z₂₅), and Si(Z₂₆)(Z₂₇),

Z₂₁ to Z₂₇ may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

e2 may be 1 or 2, e3 may be an integer selected from 1, 2, and 3, e4 may be an integer selected from 1, 2, 3, and 4, e5 may be an integer selected from 1, 2, 3, 4, and 5, e6 may be an integer selected from 1, 2, 3, 4, 5, and 6, e7 may be an integer selected from 1, 2, 3, 4, 5, 6, and 7, e9 may be an integer selected from 1, 2, 3, 4, 5, 6, 7, 8, and 9, and * indicates a binding site to a neighboring atom.

In some embodiments, Ar₁ in Formula 1 may be selected from Formulae 6-1 to 6-43, but is not limited thereto:

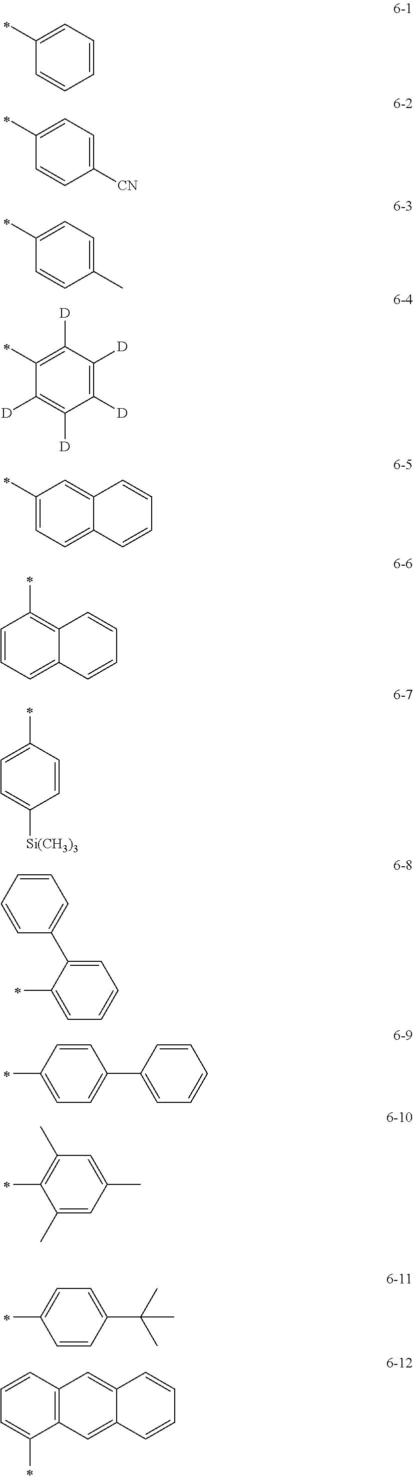

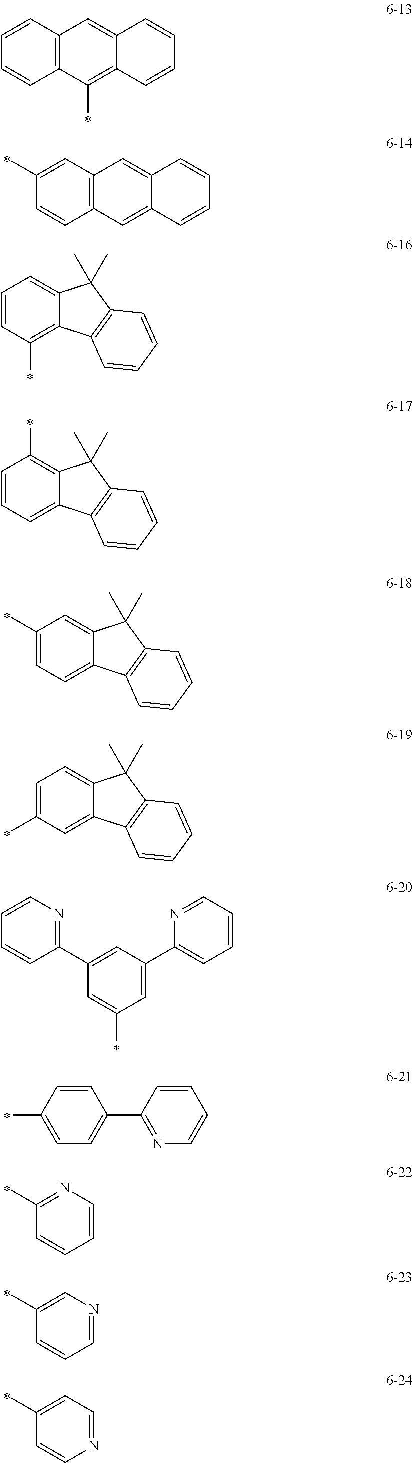

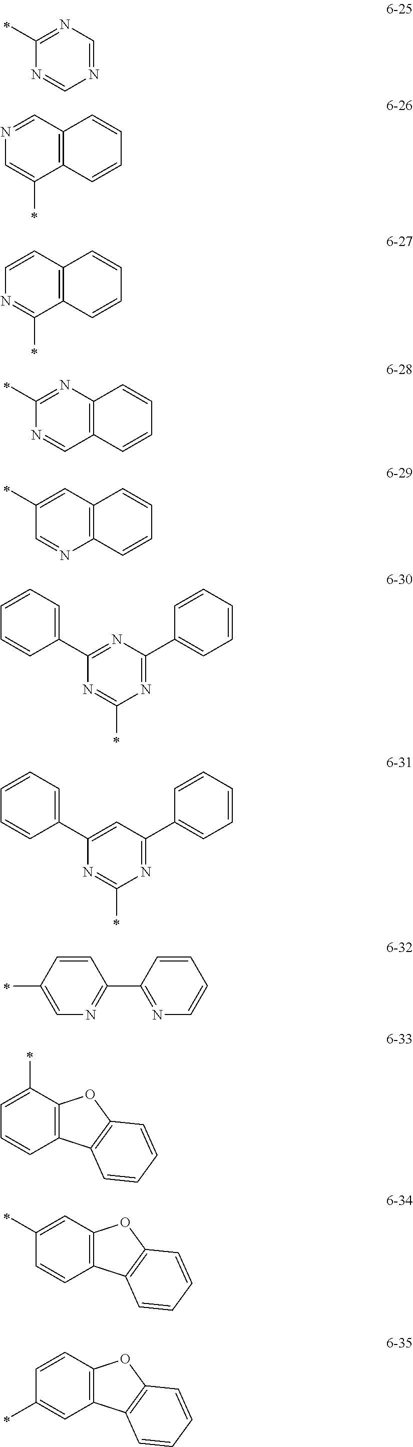

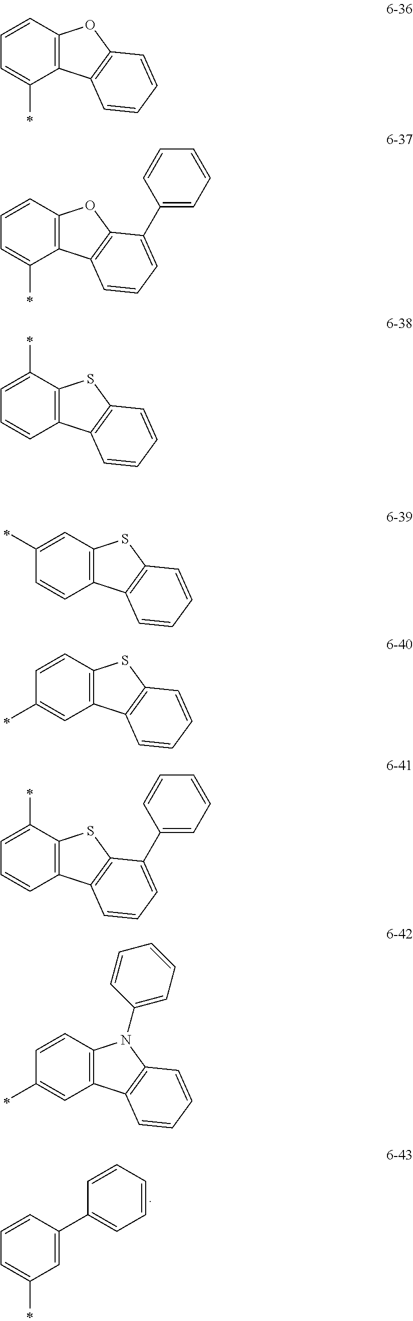

* in Formulae 6-1 to 6-43 indicates a binding site to a neighboring atom.

b1 in Formula 1 may be selected from 1, 2, and 3. b1 indicates the number of Ar₁ in Formula 1, and when b1 is 2 or more, two or more Ar₁(s) may be identical to or different from each other. In some embodiments, b1 may be 1 or 2. For example, b1 in Formula 1 may be 1.

R₁₁ to R₁₄ in Formula 1 may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), and N(Q₆)(Q₇), where Q₁ to Q₇ may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

For example, R₁₁ to R₁₄ in Formula 1 may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), and N(Q₆)(Q₇), where Q₁ to Q₇ may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

In some embodiments, R₁₁ to R₁₄ in Formula 1 may be each independently selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;

a phenyl group, a naphthyl group, a fluorenyl group, a spiro-fluorenyl group, a benzofluorenyl group, a dibenzofluorenyl group, a phenanthrenyl group, an anthracenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a carbazolyl group, a benzoimidazolyl group, a benzofuranyl group, a benzothiophenyl group, an isobenzothiazolyl group, a benzoxazolyl group, an isobenzoxazolyl group, an oxadiazolyl group, a triazinyl group, a dibenzofuranyl group, a dibenzothiophenyl group, an imidazopyridinyl group, and an imidazopyrimidinyl group;

a phenyl group, a naphthyl group, a fluorenyl group, a spiro-fluorenyl group, a benzofluorenyl group, a dibenzofluorenyl group, a phenanthrenyl group, an anthracenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a carbazolyl group, a benzoimidazolyl group, a benzofuranyl group, a benzothiophenyl group, an isobenzothiazolyl group, a benzoxazolyl group, an isobenzoxazolyl group, an oxadiazolyl group, a triazinyl group, a dibenzofuranyl group, a dibenzothiophenyl group, an imidazopyridinyl group, and an imidazopyrimidinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a carbazolyl group, a benzoimidazolyl group, a benzofuranyl group, a benzothiophenyl group, an isobenzothiazolyl group, a benzoxazolyl group, an isobenzoxazolyl group, an oxadiazolyl group, a triazinyl group, a dibenzofuranyl group, a dibenzothiophenyl group, an imidazopyridinyl group, an imidazopyrimidinyl group, and —Si(Q₃₁)(Q₃₂)(Q₃₃); and

—Si(Q₁)(Q₂)(Q₃),

where Q₁ to Q₃ and Q₃₁ to Q₃₃ may be each independently selected from a C₁-C₁₀ alkyl group, a C₁-C₁₀ alkoxy group, a phenyl group, and a naphthyl group.

In some embodiments, R₁₁ to R₁₄ in Formula 1 may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, —Si(Q₁)(Q₂)(Q₃), and groups represented by Formulae 7-1 to 7-18, where Q₁ to Q₃ may be each independently selected from a C₁-C₁₀ alkyl group, a C₁-C₁₀ alkoxy group, a phenyl group, and a naphthyl group, but embodiments of the present disclosure are not limited thereto:

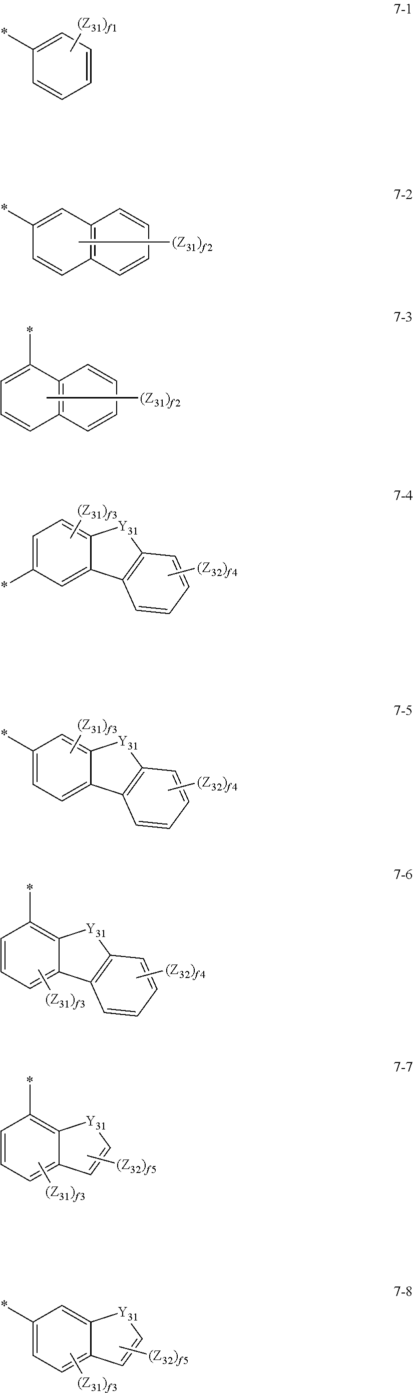

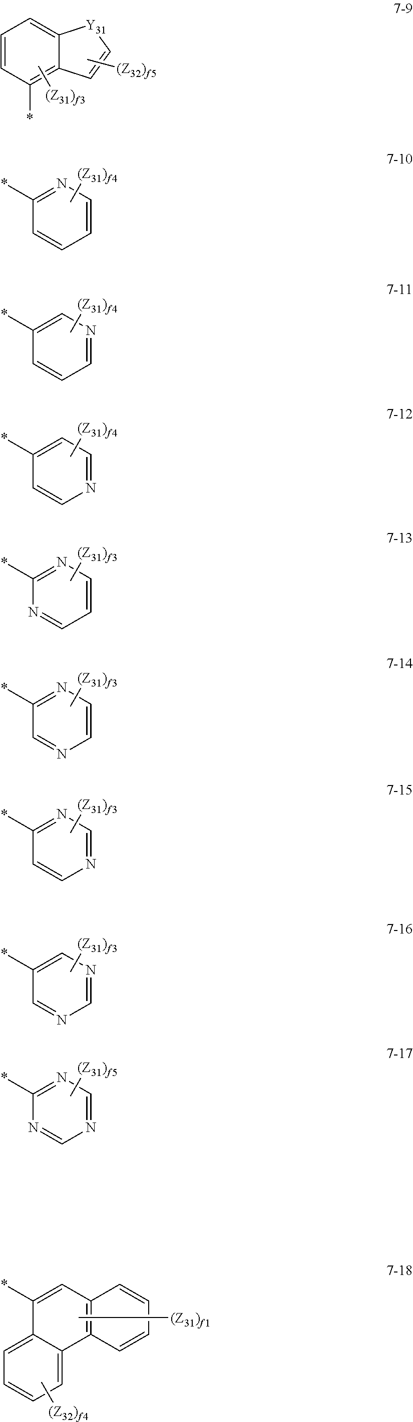

In Formulae 7-1 to 7-18,

Y₃₁ may be selected from O, S, C(Z₃₃)(Z₃₄), N(Z₃₅), and Si(Z₃₆)(Z₃₇),

Z₃₁ to Z₃₇ may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

f1 may be an integer selected from 1, 2, 3, 4, and 5, f2 may be an integer selected from 1, 2, 3, 4, 5, 6, and 7, f3 may be an integer selected from 1, 2, and 3, f4 may be an integer selected from 1, 2, 3, and 4, f5 may be 1 or 2, and

* indicates a binding site to a neighboring atom.

In some embodiments, R₁₁ to R₁₄ in Formula 1 may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, —Si(Q₁)(Q₂)(Q₃), and groups represented by Formulae 8-1 to 8-29, where Q₁ to Q₃ may be each independently selected from a C₁-C₁₀ alkyl group, a C₁-C₁₀ alkoxy group, a phenyl group, and a naphthyl group, but embodiments of the present disclosure are not limited thereto:

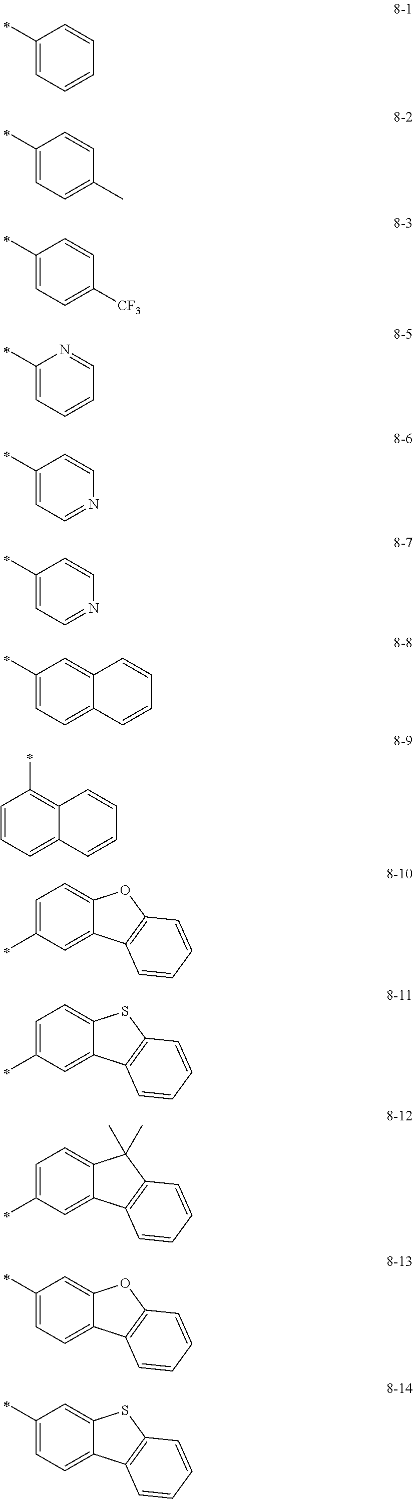

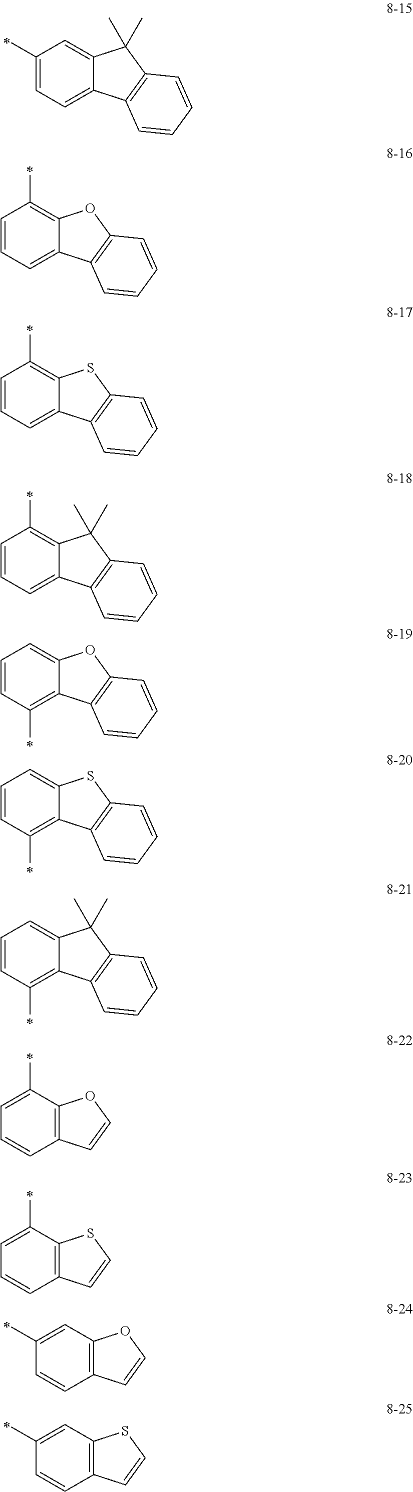

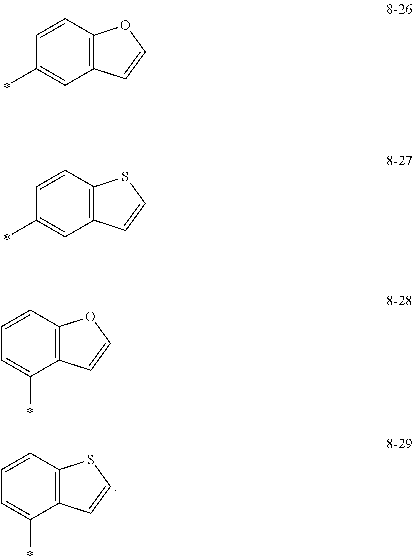

in Formulae 8-1 to 8-29, * indicates a binding site to a neighboring atom.

In some embodiments, in Formula 1, R₁₁ and R₁₂ may be each independently hydrogen or a phenyl group, and R₁₃ and R₁₄ may both be hydrogen.

b11 and b12 in Formula 1 may be each independently selected from 0, 1, 2, and 3. b11 indicates the number of R₁₁, and when b11 is 2 or more, two or more R₁₁(s) may be identical to or different from each other. For example, b11 may be 0 or 1. b12 indicates the number of R₁₂, and when b12 is 2 or more, two or more R₁₂(s) may be identical to or different from each other. For example, b12 may be 0 or 1.

c11 and c12 in Formula 1 may be each independently selected from 0, 1, 2, 3, 4, 5, and 6. c11 indicates the number of *-[(L₁₁)ₐ₁₁-(R₁₁)_{b11}], and when c11 is 2 or more, two or more *-[(L₁₁)ₐ₁₁-(R₁₁)_{b11}](s) may be identical to or different from each other. c12 indicates the number of *-[(L₁₂)ₐ₁₂-(R₁₂)_{b12}], and when c12 is 2 or more, two or more *-[(L₁₂)ₐ₁₂-(R₁₂)_{b12}](s) may be identical to or different from each other.

For example, the condensed cyclic compound represented by Formula 1 may be represented by one of Formulae 1A to 1I:

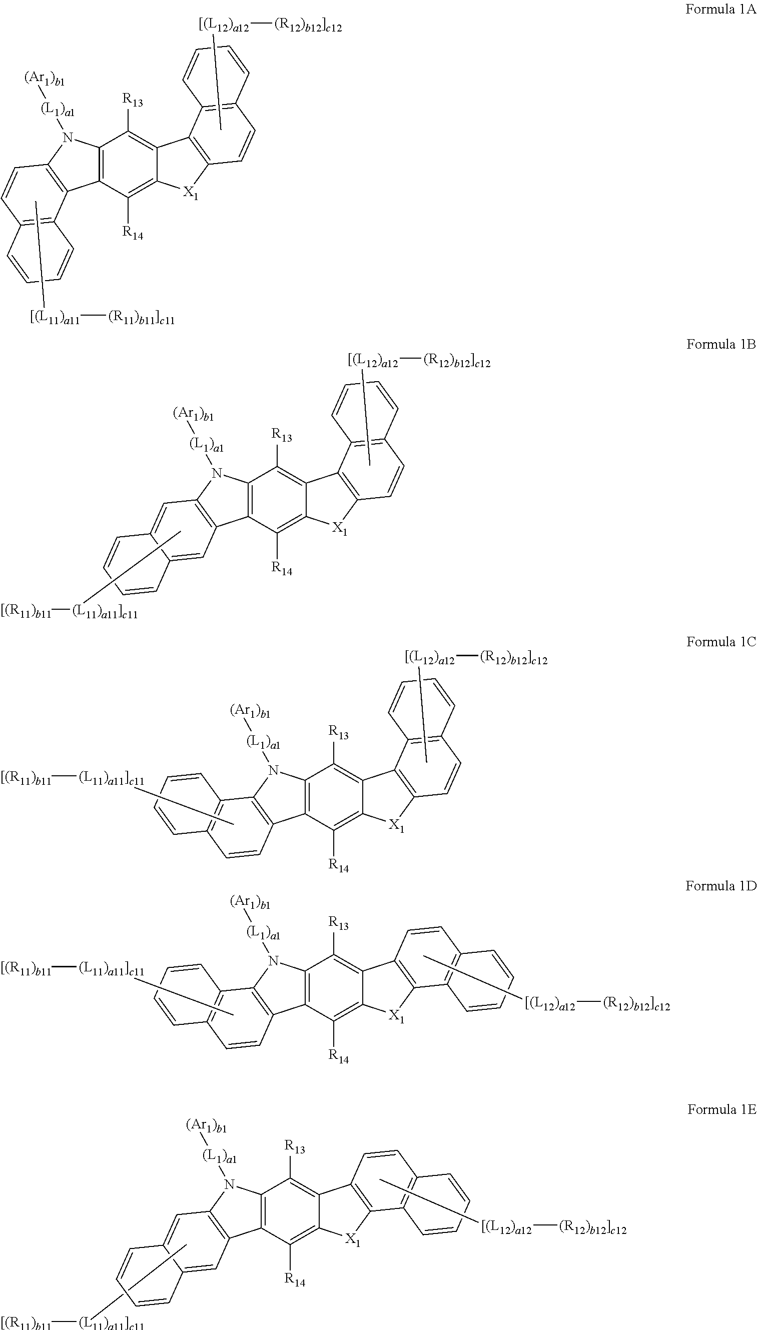

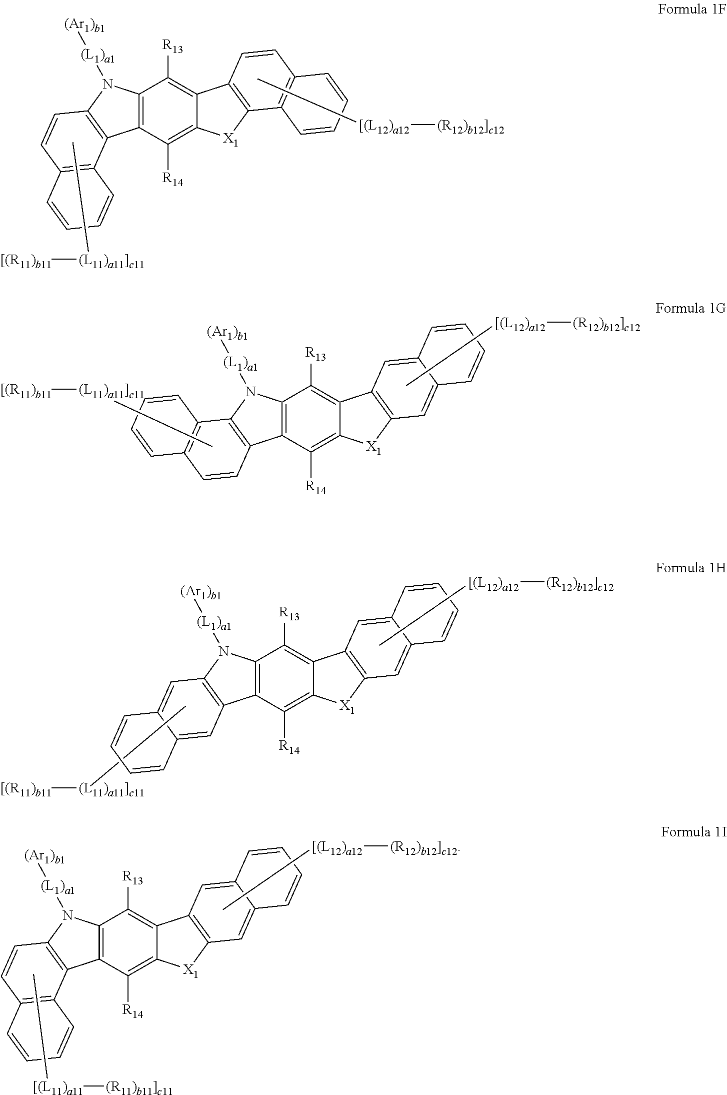

Descriptions of X₁, L₁, L₁₁, L₁₂, a1, a11, a12, Ar₁, b1, R₁₁ to R₁₄, b11, b12, c11, and c12 in Formulae 1A to 1I are the same as described above.

In some embodiments, the condensed cyclic compound represented by Formula 1 may be represented by one of Formulae 2A to 2I, but is not limited thereto:

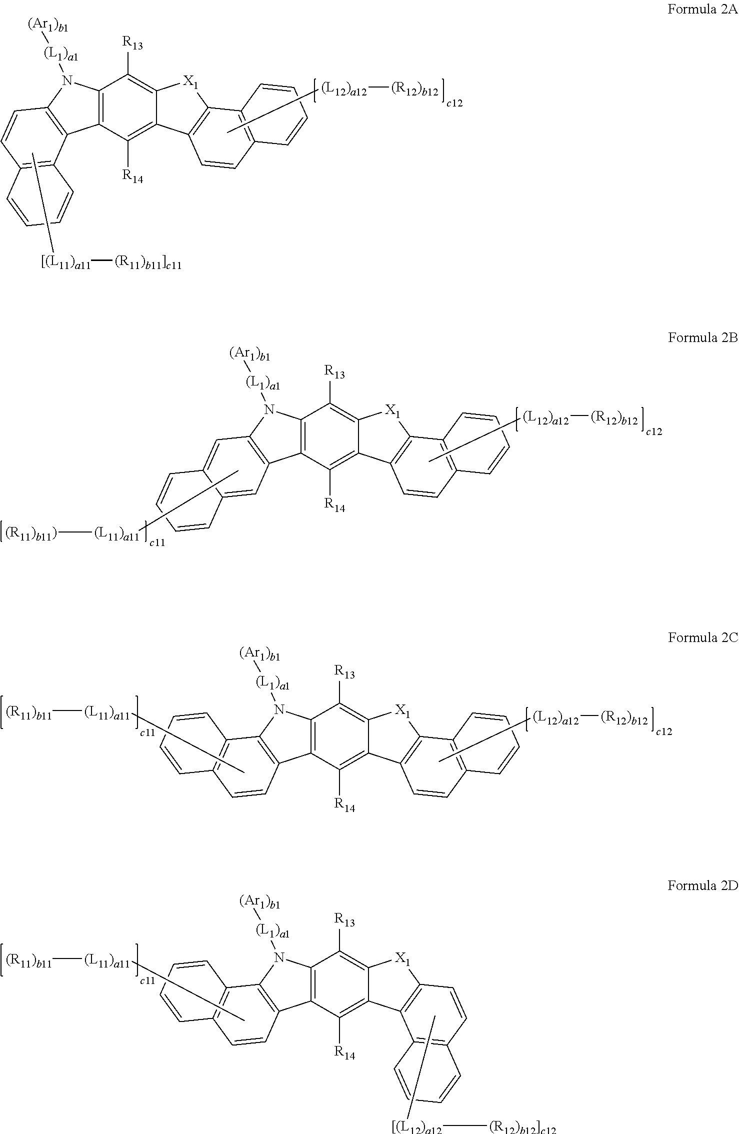

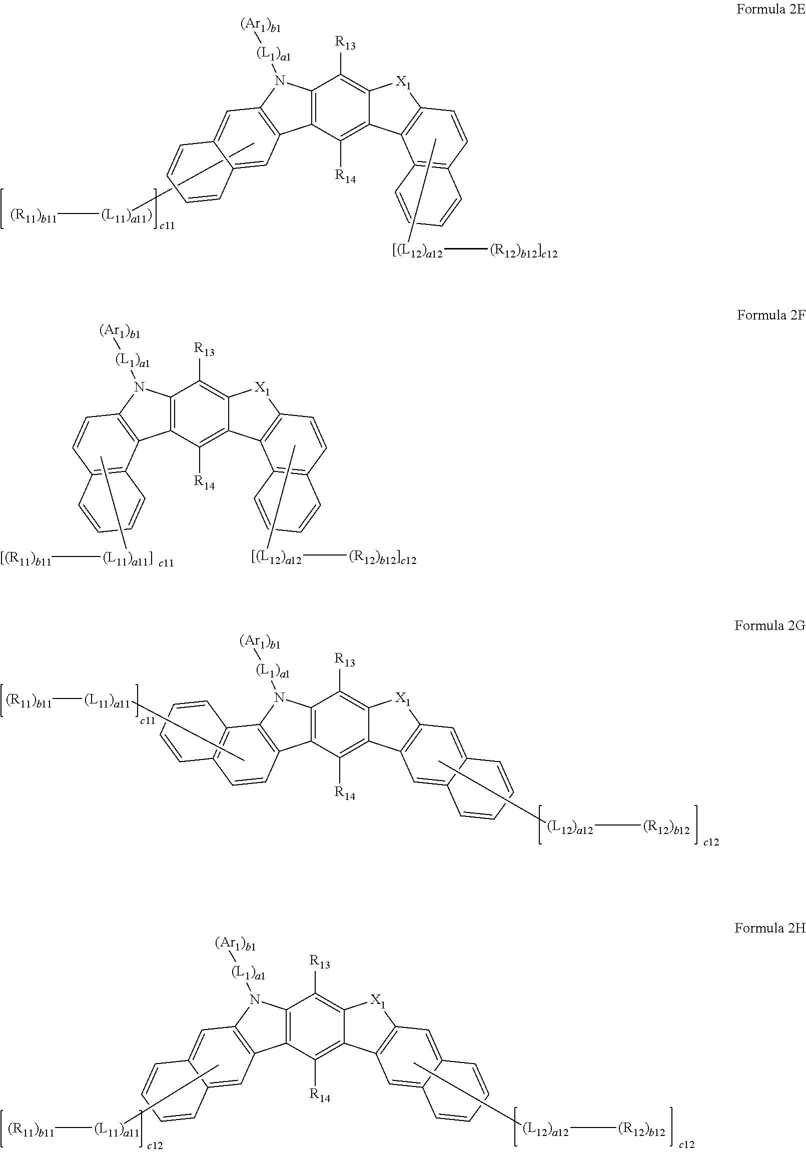

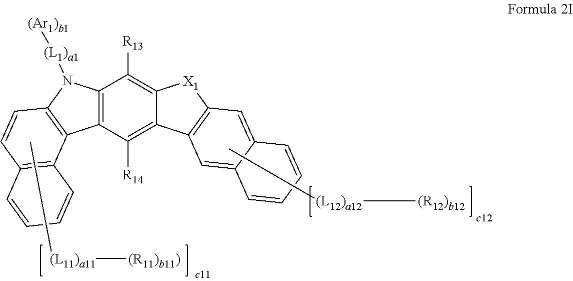

Descriptions of X₁, L₁, L₁₁, L₁₂, a1, a11, a12, Ar₁, b1, R₁₁ to R₁₄, b11, b12, c11, and c12 in Formulae 2A to 2I are the same as described above.

In some embodiments, the condensed cyclic compound may be represented by one of Formulae 1A-1 to 1A-4 and 2F-1 to 2F-4, but is not limited thereto:

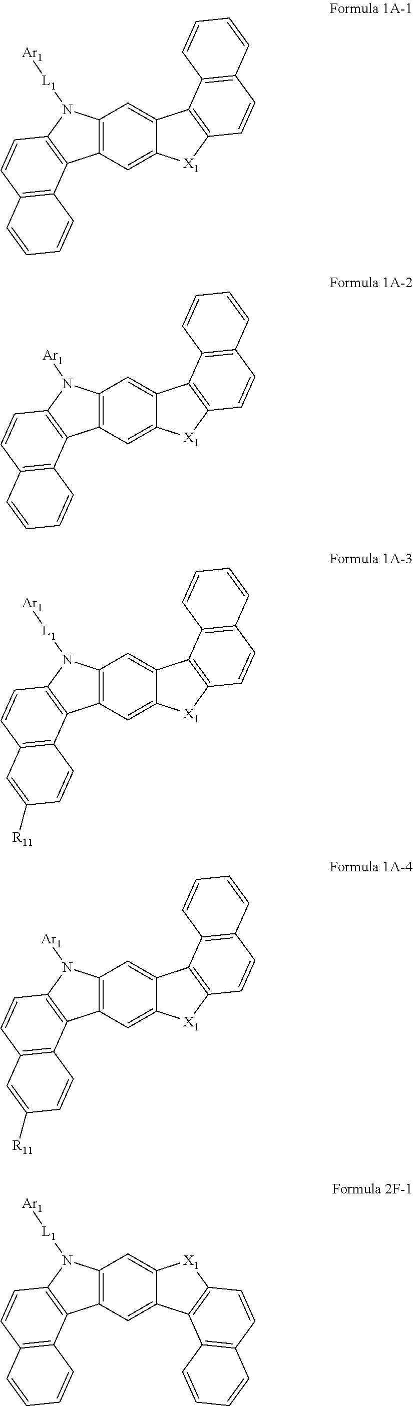

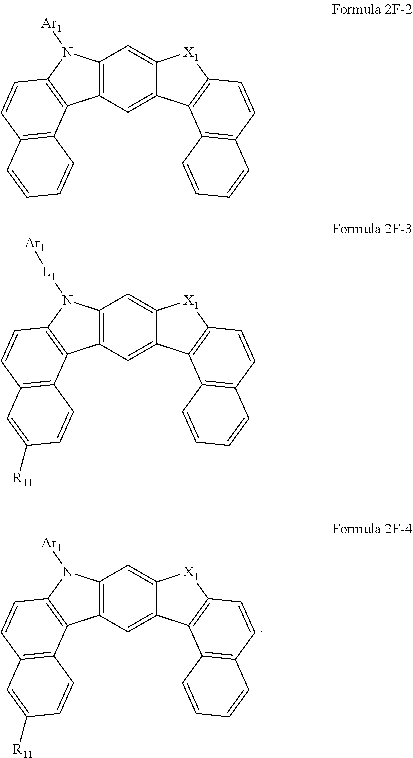

In Formulae 1A-1 to 1A-4 and 2F-1 to 2F-4,

X₁ may be O or S,

L₁ may be selected from groups represented by Formulae 4-1 to 4-25,

Ar₁ may be selected from groups represented by Formulae 6-1 to 6-43, and

R₁₁ may be selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

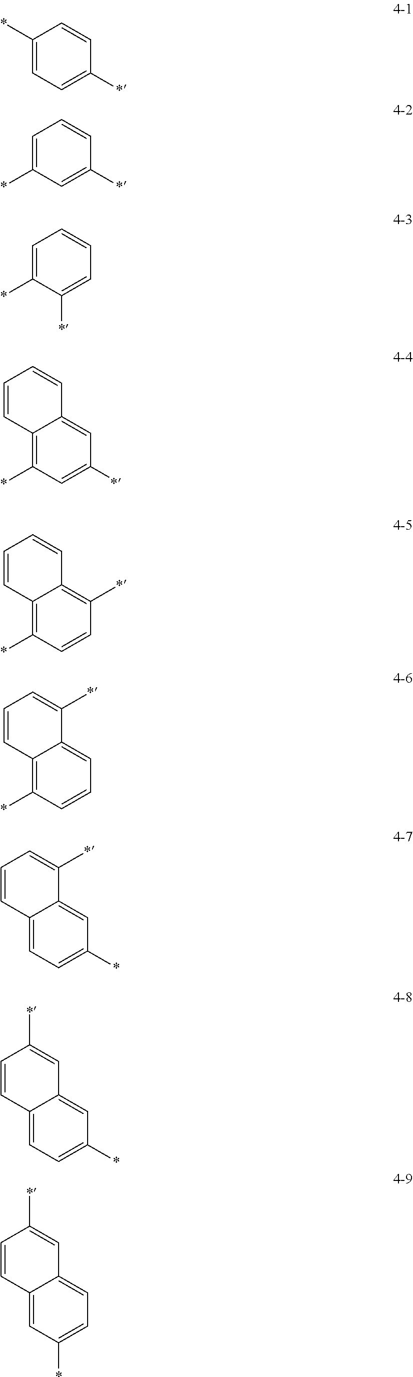

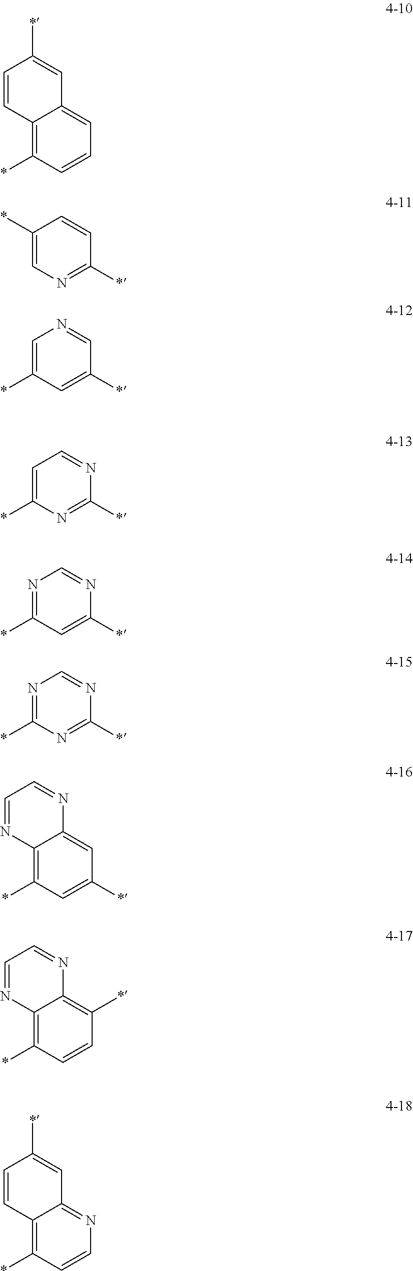

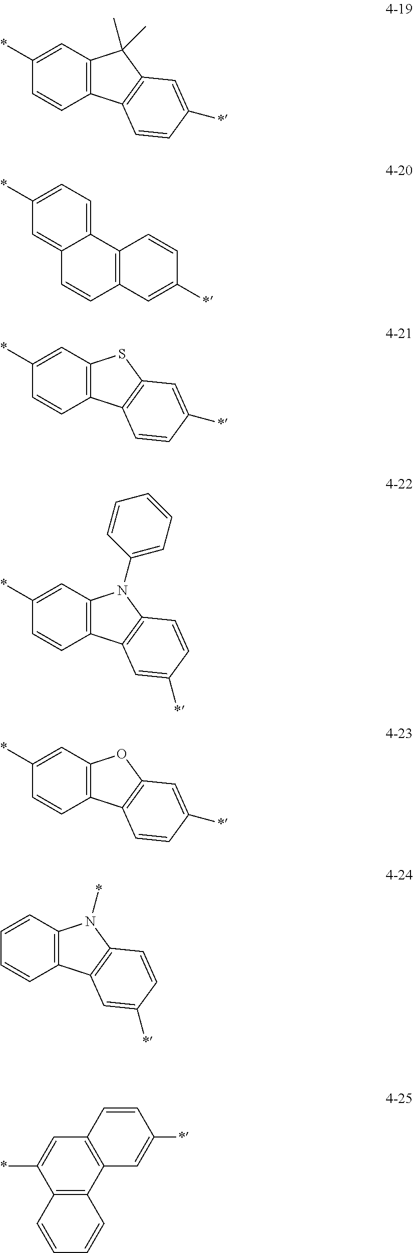

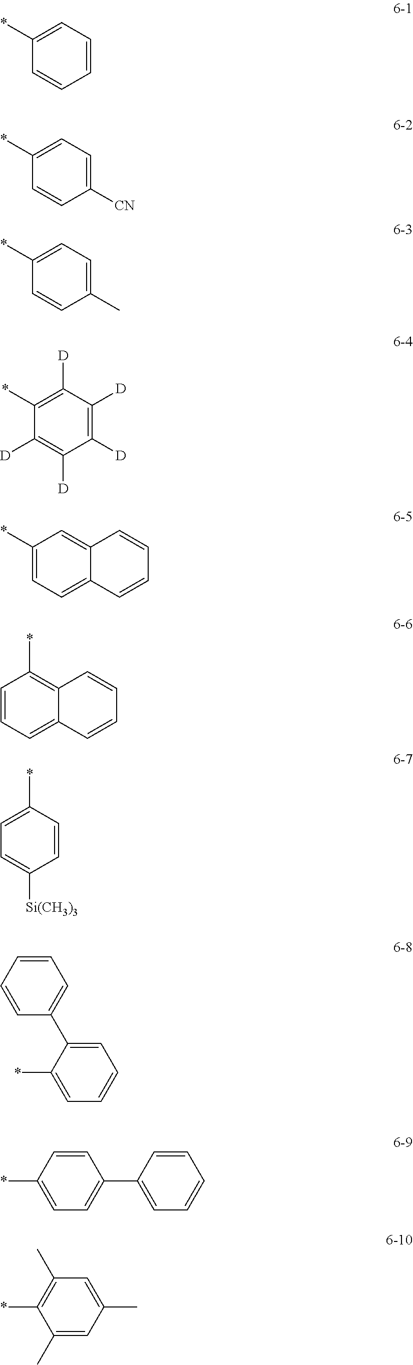

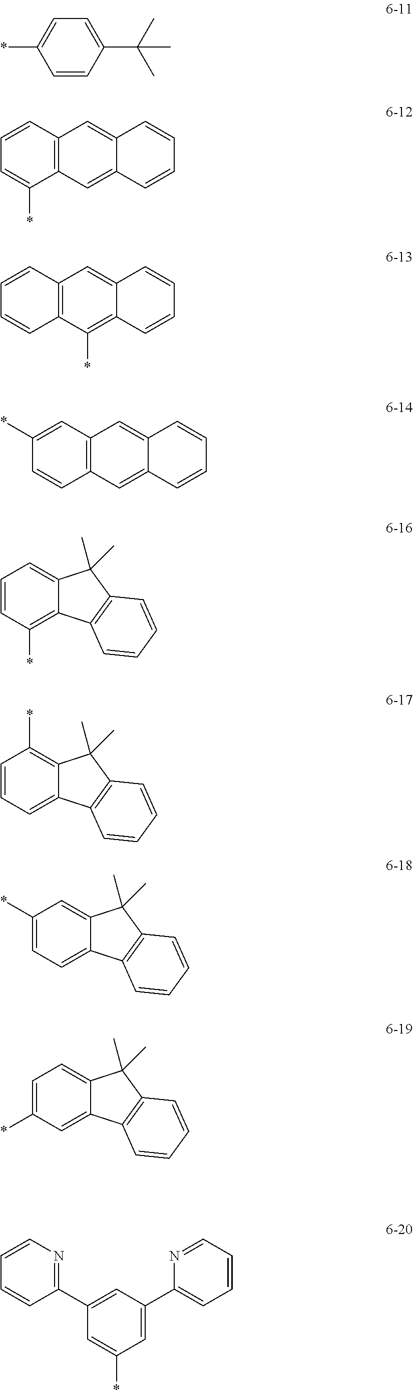

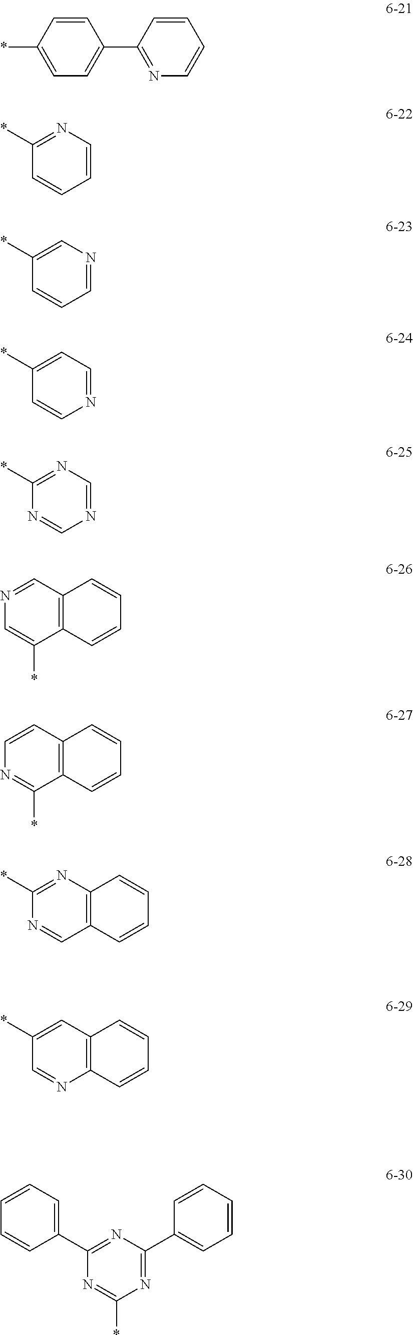

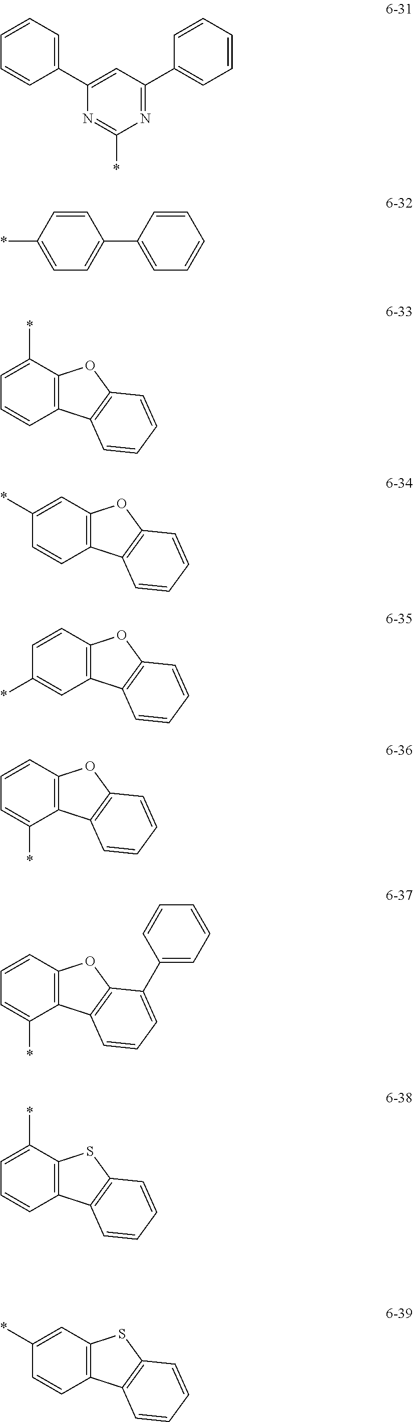

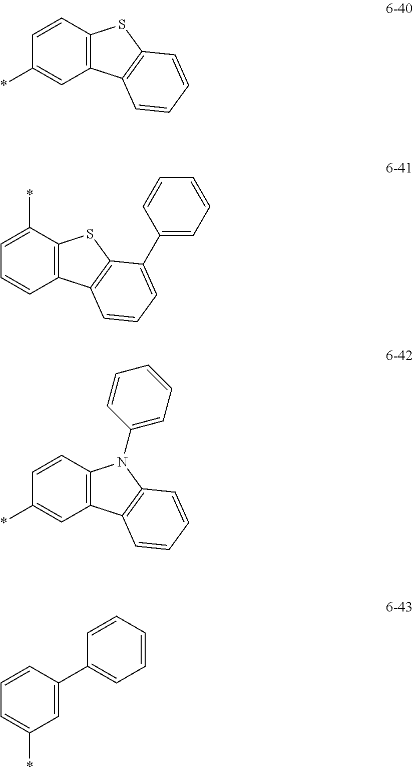

where * and *′ in Formulae 4-1 to 4-25 and Formulae 6-1 to 6-43 each indicate a binding site to a neighboring atom.

For example, the condensed cyclic compound represented by Formula 1 may be one of Compounds 1 to 32, but is not limited thereto:

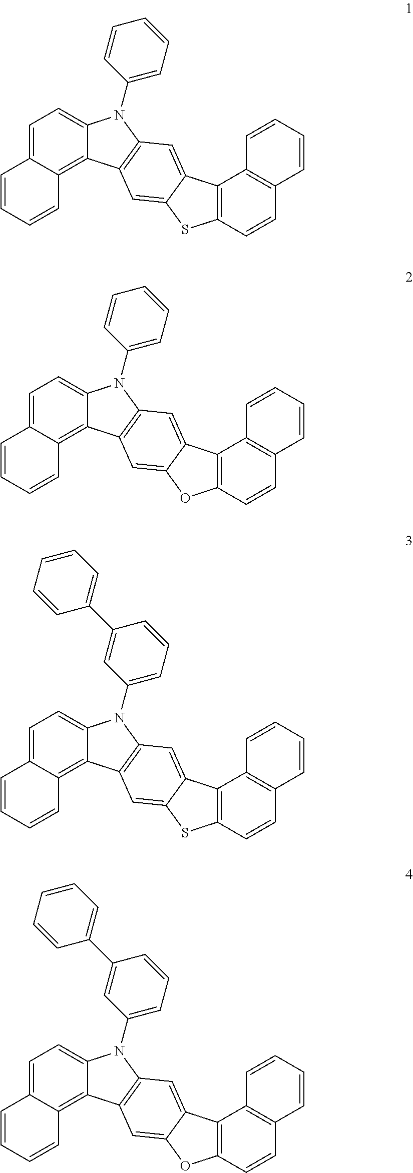 [SMILES: c1ccc(-n2c3cc4c(cc3c3c5ccccc5ccc32)sc2ccc3ccccc3c24)cc1 ; c1ccc(-n2c3cc4c(cc3c3c5ccccc5ccc32)oc2ccc3ccccc3c24)cc1 ; c1ccc(-c2cccc(-n3c4cc5c(cc4c4c6ccccc6ccc43)sc3ccc4ccccc4c35)c2)cc1 ; c1ccc(-c2cccc(-n3c4cc5c(cc4c4c6ccccc6ccc43)oc3ccc4ccccc4c35)c2)cc1]

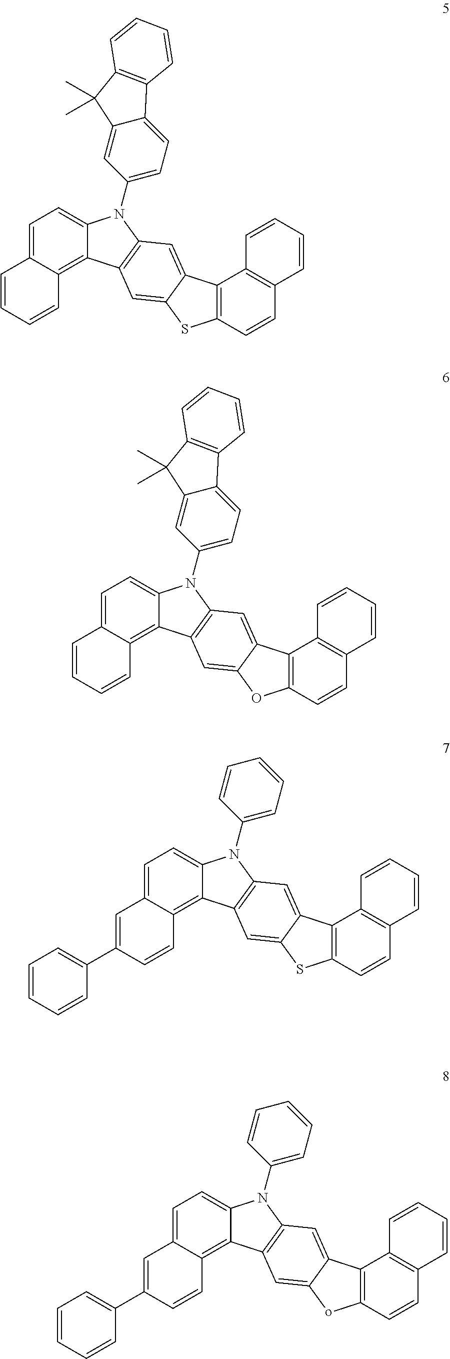

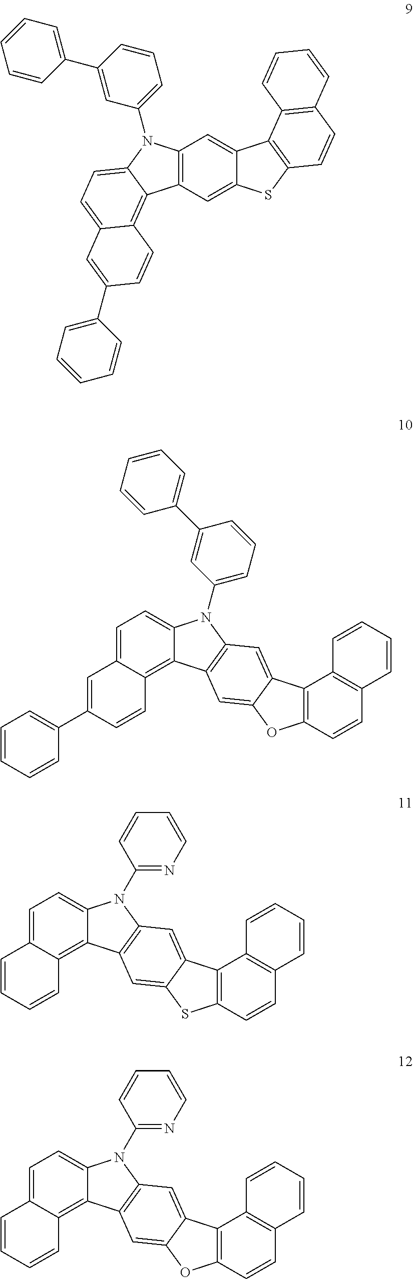 [SMILES: c1ccc(-c2cccc(-n3c4cc5c(cc4c4c6ccc(-c7ccccc7)cc6ccc43)sc3ccc4ccccc4c35)c2)cc1 ; c1ccc(-c2cccc(-n3c4cc5c(cc4c4c6ccc(-c7ccccc7)cc6ccc43)oc3ccc4ccccc4c35)c2)cc1 ; c1ccc(-n2c3cc4c(cc3c3c5ccccc5ccc32)sc2ccc3ccccc3c24)nc1 ; c1ccc(-n2c3cc4c(cc3c3c5ccccc5ccc32)oc2ccc3ccccc3c24)nc1]

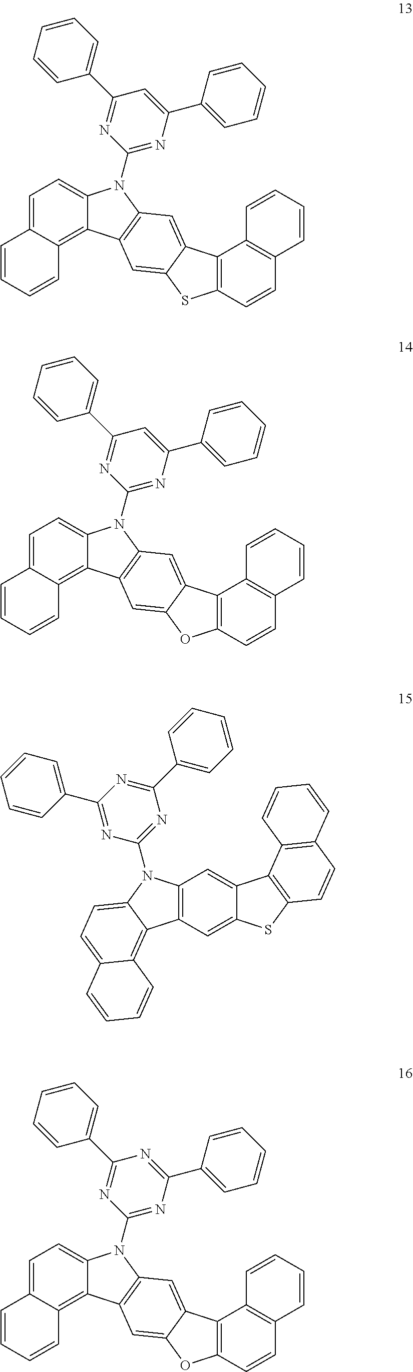 [SMILES: c1ccc(-c2cc(-c3ccccc3)nc(-n3c4cc5c(cc4c4c6ccccc6ccc43)sc3ccc4ccccc4c35)n2)cc1 ; c1ccc(-c2cc(-c3ccccc3)nc(-n3c4cc5c(cc4c4c6ccccc6ccc43)oc3ccc4ccccc4c35)n2)cc1 ; c1ccc(-c2nc(-c3ccccc3)nc(-n3c4cc5c(cc4c4c6ccccc6ccc43)sc3ccc4ccccc4c35)n2)cc1 ; c1ccc(-c2nc(-c3ccccc3)nc(-n3c4cc5c(cc4c4c6ccccc6ccc43)oc3ccc4ccccc4c35)n2)cc1]

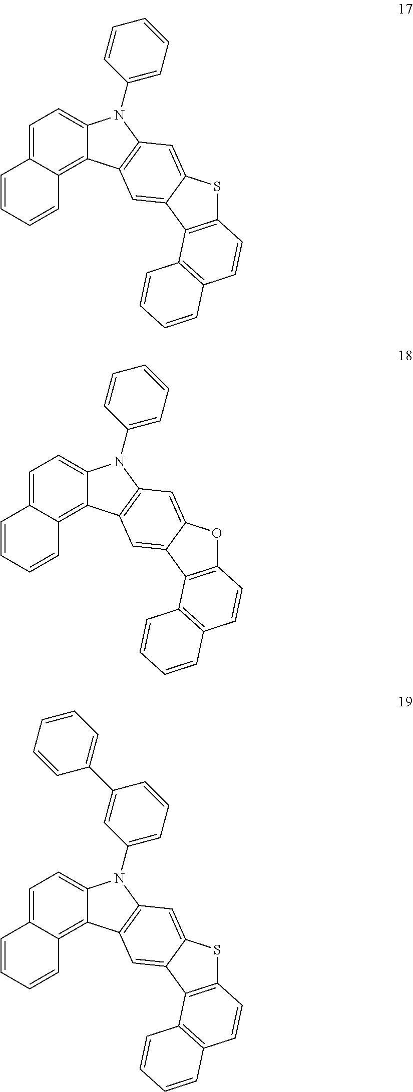

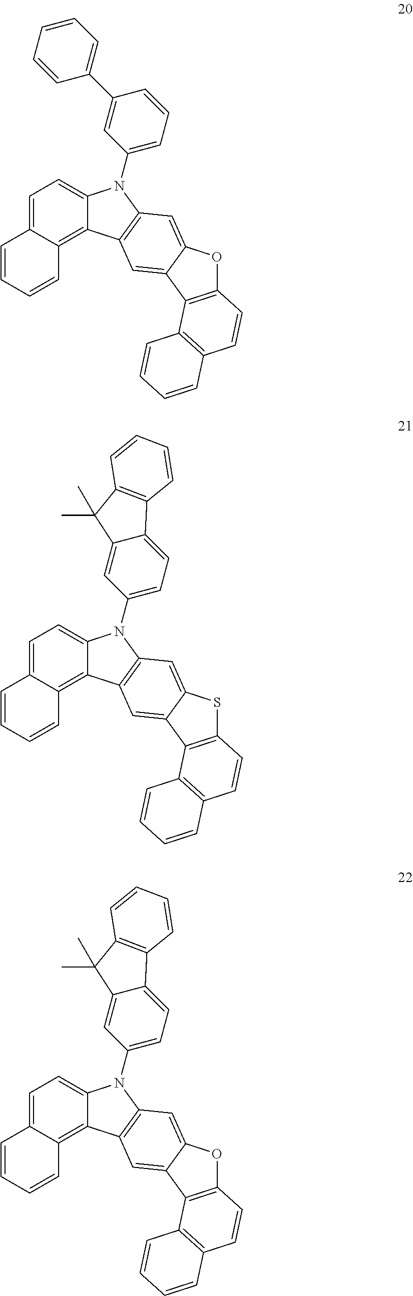

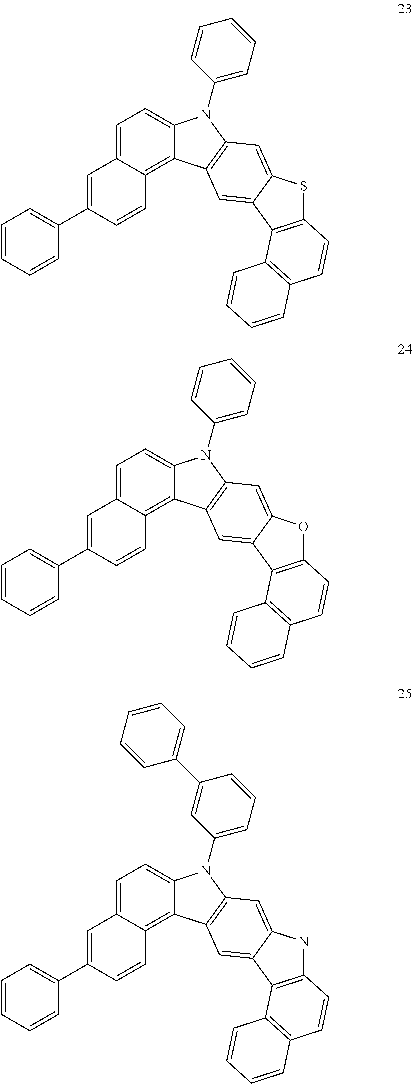

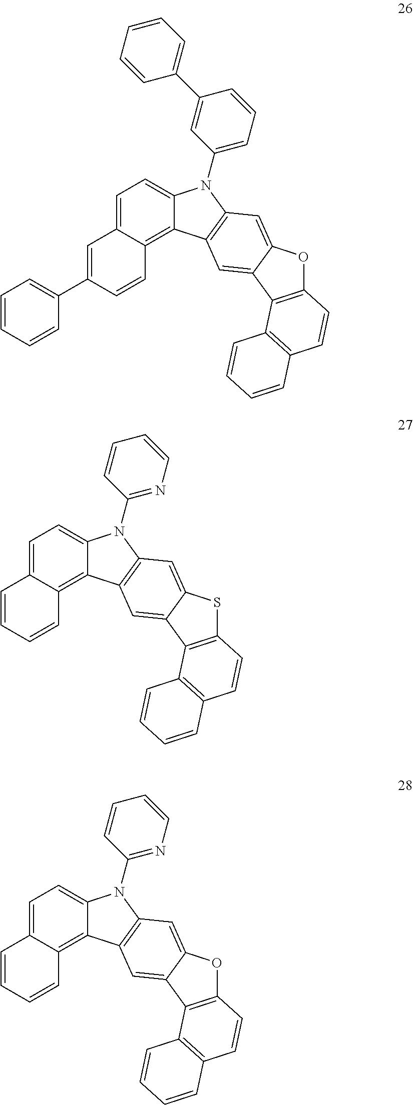 [SMILES: c1ccc(-c2cccc(-n3c4cc5oc6ccc7ccccc7c6c5cc4c4c5ccc(-c6ccccc6)cc5ccc43)c2)cc1 ; c1ccc(-n2c3cc4sc5ccc6ccccc6c5c4cc3c3c4ccccc4ccc32)nc1 ; c1ccc(-n2c3cc4oc5ccc6ccccc6c5c4cc3c3c4ccccc4ccc32)nc1]

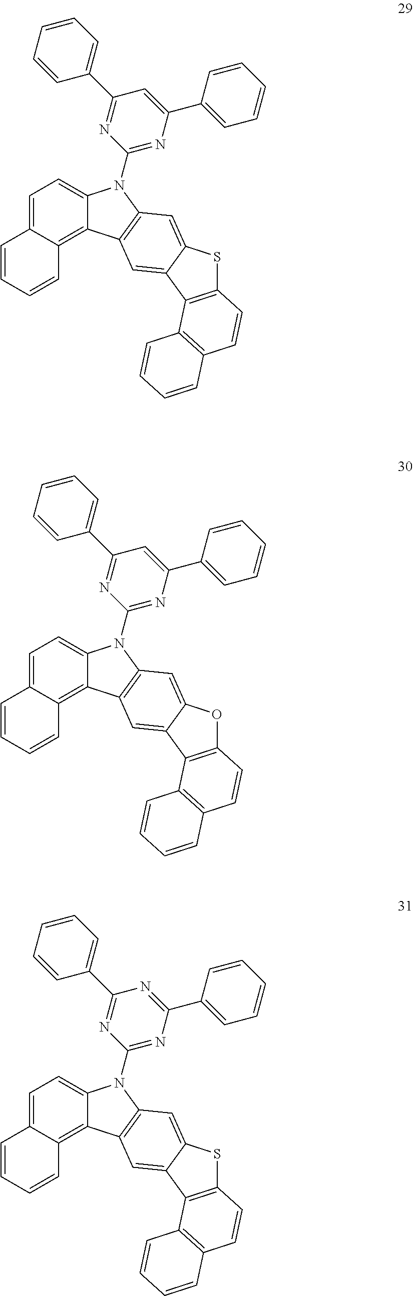 [SMILES: c1ccc(-c2cc(-c3ccccc3)nc(-n3c4cc5sc6ccc7ccccc7c6c5cc4c4c5ccccc5ccc43)n2)cc1 ; c1ccc(-c2cc(-c3ccccc3)nc(-n3c4cc5oc6ccc7ccccc7c6c5cc4c4c5ccccc5ccc43)n2)cc1 ; c1ccc(-c2nc(-c3ccccc3)nc(-n3c4cc5sc6ccc7ccccc7c6c5cc4c4c5ccccc5ccc43)n2)cc1]

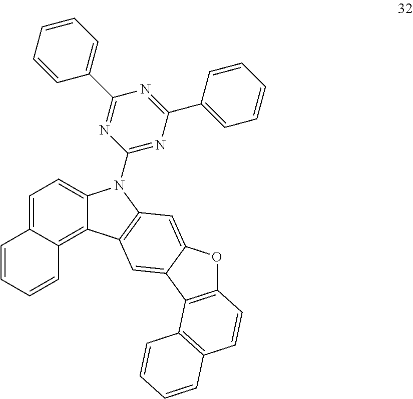 [SMILES: c1ccc(-c2nc(-c3ccccc3)nc(-n3c4cc5oc6ccc7ccccc7c6c5cc4c4c5ccccc5ccc43)n2)cc1]

The condensed cyclic compound represented by Formula 1 may be synthesized by using one or more suitable organic synthetic methods. Suitable synthesis methods of the condensed cyclic compound according to embodiments of the present disclosure should be apparent to those of ordinary skill in the art in view of embodiments provided below.

At least one condensed cyclic compound represented by Formula 1 may be used between a pair of electrodes of an organic light-emitting device. For example, the condensed cyclic compound may be included in an emission layer. For example, an organic light-emitting device according to an embodiment of the present disclosure may include: a first electrode; a second electrode facing the first electrode; and an organic layer between the first electrode and the second electrode, the organic layer including an emission layer, where the organic layer further includes at least one condensed cyclic compound represented by Formula 1.

The expression, “(the organic layer) includes at least one condensed cyclic compound” used herein may be interpreted as “(the organic layer) includes one condensed cyclic compound of Formula 1 or at least two different condensed cyclic compounds of Formula 1.”

For example, the organic layer may include only Compound 1 as the condensed cyclic compound. For example, Compound 1 may be present in the emission layer of the organic light-emitting device. Alternatively, the organic layer may include Compound 1 and Compound 2 as the condensed cyclic compounds. For example, Compound 1 and Compound 2 may be present in the same layer (for example, both Compound 1 and Compound 2 may be present in the emission layer).

The organic layer may include i) a hole transport region between the first electrode (e.g., an anode) and the emission layer, the hole transport region including at least one selected from a hole injection layer, a hole transport layer, a buffer layer, and an electron blocking layer, and may further include ii) an electron transport region between the emission layer and the second electrode (e.g, a cathode), the electron transport region including at least one selected from a hole blocking layer, an electron transport layer, and an electron injection layer. The condensed cyclic compound represented by Formula 1 may be included in the emission layer.

For example, the emission layer may include at least one condensed cyclic compound represented by Formula 1. In some embodiments, the emission layer may include at least one condensed cyclic compound represented by Formula 1, and may further include a dopant. The condensed cyclic compound may serve as a host in the emission layer, and an amount of the condensed cyclic compound in the emission layer may be greater than that of the dopant in the emission layer.

The term “organic layer” used herein refers to a single layer and/or a plurality of layers positioned between the first electrode and the second electrode of the organic light-emitting device. A material included in the “organic layer” is not limited to an organic material.

The drawing is a schematic cross-sectional view of an organic light-emitting device 10 according to one or more embodiments of the present disclosure. The organic light-emitting device 10 includes a first electrode 110, an organic layer 150, and a second electrode 190.

Hereinafter, the structure of an organic light-emitting device according to one or more embodiments of the present disclosure and a method of manufacturing an organic light-emitting device according to one or more embodiments will be described with reference to the drawing.

In the drawing, a substrate may be additionally positioned under the first electrode 110 or on the second electrode 190. The substrate may be a glass or transparent plastic substrate with excellent mechanical strength, thermal stability, transparency, surface smoothness, ease of handling, and/or water-resistance.

For example, the first electrode 110 may be formed by depositing or sputtering a material for forming a first electrode on the substrate. When the first electrode 110 is an anode, the material for forming the first electrode may be selected from materials having a high work function to facilitate hole injection. The first electrode 110 may be a reflective electrode, a semi-transmissive electrode, or a transmissive electrode. The material for forming the first electrode may be a transparent and highly conductive material, and non-limiting examples thereof include indium tin oxide (ITO), indium zinc oxide (IZO), tin oxide (SnO₂), and zinc oxide (ZnO). Alternatively, in order to form the first electrode 110 that is a semi-transmissive electrode or a reflective electrode, at least one selected from magnesium (Mg), aluminum (Al), aluminum-lithium (Al—Li), calcium (Ca), magnesium-indium (Mg—In), and magnesium-silver (Mg—Ag) may be used as the material for forming the first electrode.

The first electrode 110 may have a single-layer structure, or a multi-layer structure including two or more layers. For example, the first electrode 110 may have a three-layered structure of ITO/Ag/ITO, but the structure of the first electrode 110 is not limited thereto.

The organic layer 150 may be positioned on the first electrode 110. The organic layer 150 may include an emission layer.

The organic layer 150 may further include a hole transport region between the first electrode 110 and the emission layer, and an electron transport region between the emission layer and the second electrode 190.

The hole transport region may include at least one selected from a hole injection layer (HIL), a hole transport layer (HTL), a buffer layer, and an electron blocking layer (EBL), and the electron transport region may include at least one selected from a hole blocking layer (HBL), an electron transport layer (ETL), and an electron injection layer (EIL). However, embodiments of the present disclosure are not limited thereto.

The hole transport region may have a single-layer structure formed of a single material, a single-layer structure formed of a plurality of different materials, or a multi-layer structure having a plurality of layers formed of a plurality of different materials.

For example, the hole transport region may have a single-layer structure formed of a plurality of different materials or may have a structure of HIL/HTL, a structure of HIL/HTL/buffer layer, a structure of HIL/buffer layer, a structure of HTL/buffer layer, or a structure of HIL/HTL/EBL, where the layers of each structure are sequentially stacked from the first electrode 110 in this stated order. However, the structure of the hole transport region is not limited thereto.

When the hole transport region includes an HIL, the HIL may be formed on the first electrode 110 by using one or more suitable methods, such as vacuum deposition, spin coating, casting, an Langmuir-Blodgett (LB) method, inkjet printing, laser printing, and/or laser induced thermal imaging (LITI).

When the HIL is formed by vacuum deposition, the vacuum deposition, for example, may be performed at a deposition temperature of about 100 to about 500° C., at a vacuum degree of about 10⁻⁸ to about 10⁻³ torr, and at a deposition rate of about 0.01 to about 100 Å/sec, in consideration of the compound for forming the HIL to be deposited and the structure of the HIL to be formed.

When the HIL is formed by spin coating, the spin coating may be performed at a coating rate of about 2000 rpm to about 5000 rpm and at a heat treatment temperature of about 80° C. to 200° C., in consideration of the compound for forming the HIL to be deposited and the structure of the HIL to be formed.

When the hole transport region includes an HTL, the HTL may be formed on the first electrode 110 or on the HIL by using one or more suitable methods, such as vacuum deposition, spin coating, casting, an LB method, inkjet printing, laser printing, and/or LITI. When the HTL is formed by vacuum deposition and/or spin coating, deposition and coating conditions for the HTL may be similar to the deposition and coating conditions for the HIL.

The hole transport region may include the condensed cyclic compound represented by Formula 1. For example, the hole transport region may include an HTL, and the condensed cyclic compound represented by Formula 1 may be included in the HTL.

In some embodiments, the hole transport region may include at least one selected from m-MTDATA, TDATA, 2-TNATA, NPB, β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (Pani/DBSA), poly(3,4-ethylenedioxythiophene)/poly(4-styrenesulfonate) (PEDOT/PSS), polyaniline/camphor sulfonicacid (Pani/CSA), (polyaniline)/poly(4-styrenesulfonate) (PANI/PSS), a compound represented by Formula 201, and a compound represented by Formula 202:

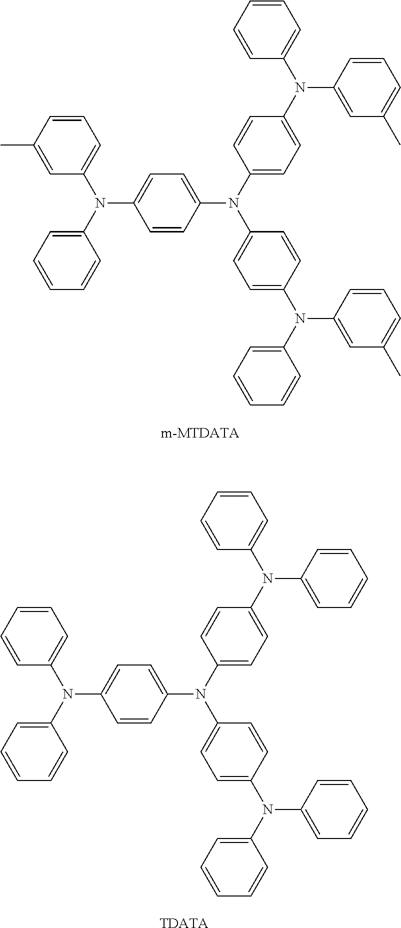

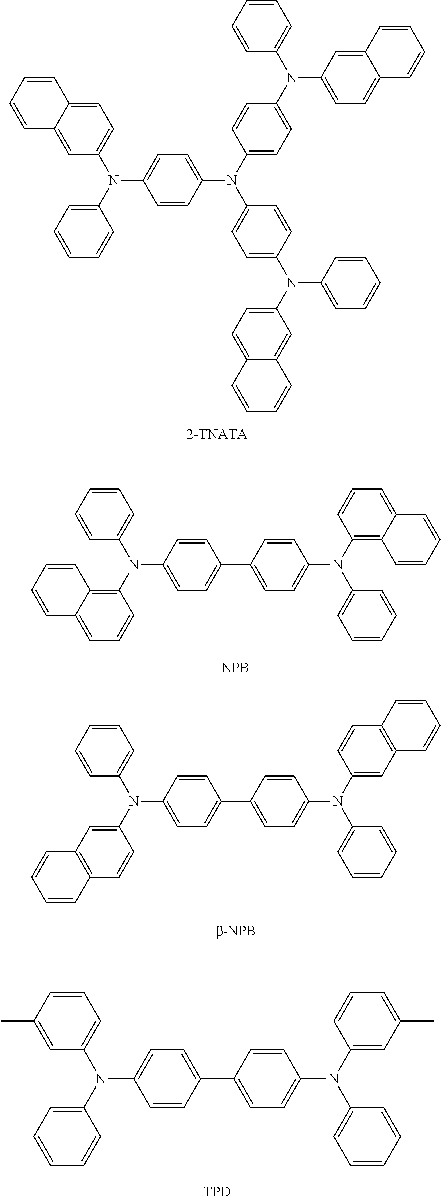

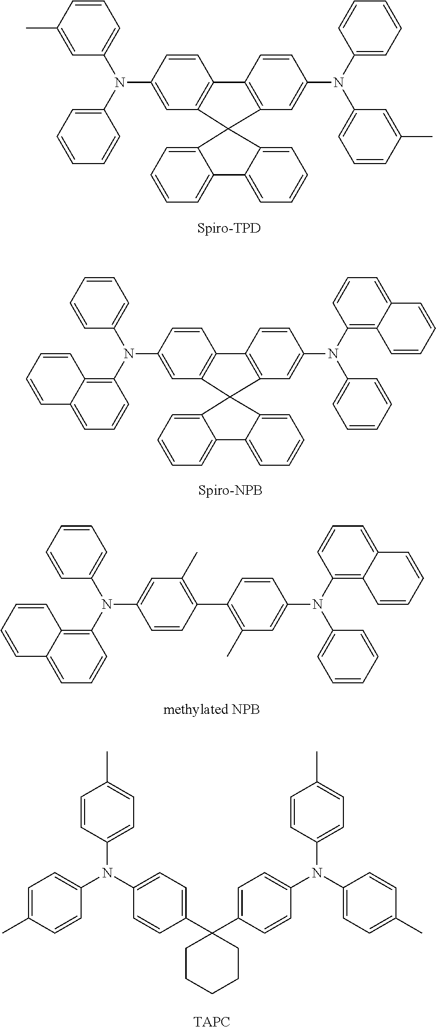

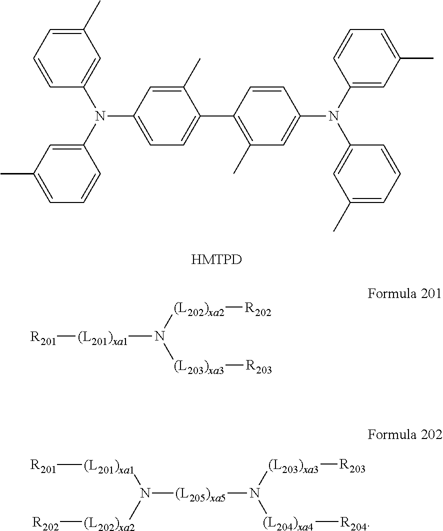

In Formulae 201 and 202,

descriptions of L₂₀₁ to L₂₀₅ may be each independently the same as provided herein in connection with L₁,

xa1 to xa4 may be each independently selected from 0, 1, 2, and 3,

xa5 may be selected from 1, 2, 3, 4, and 5, and

R₂₀₁ to R₂₀₄ may be each independently selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

For example, in Formulae 201 and 202,

L₂₀₁ to L₂₀₅ may be each independently selected from:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, an isoindolyl group, a quinolinyl group, an isoquinolinyl group, a quinoxalinyl group, a quinazolinyl group, a carbazolyl group, and a triazinyl group,

xa1 to xa4 may be each independently selected from 0, 1, and 2,

xa5 may be 1, 2, or 3, and

R₂₀₁ to R₂₀₄ may be each independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an azulenyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, but embodiments of the present disclosure are not limited thereto.

The compound represented by Formula 201 may be represented by Formula 201A:

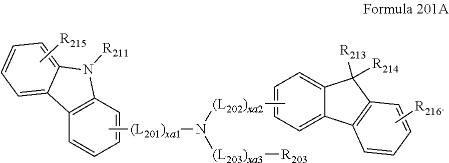

For example, the compound represented by Formula 201 may be represented by Formula 201A-1, but is not limited thereto:

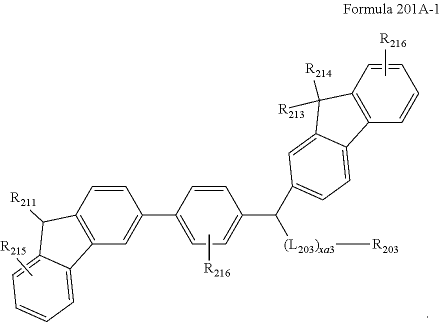

The compound represented by Formula 202 may be represented by Formula 202A, but is not limited thereto:

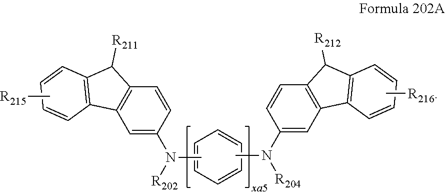

In Formulae 201A, 201A-1, and 202A, descriptions of L₂₀₁ to L₂₀₃, xa1 to xa3, xa5, and R₂₀₂ to R₂₀₄ may be the same as described above, descriptions of R₂₁₁ and R₂₁₂ may be each independently the same as provided herein in connection with R₂₀₃, and R₂₁₃ to R₂₁₆ may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

The compound represented by Formula 201 and the compound represented by Formula 202 may each independently include Compounds HT1 to HT20, but are not limited thereto.

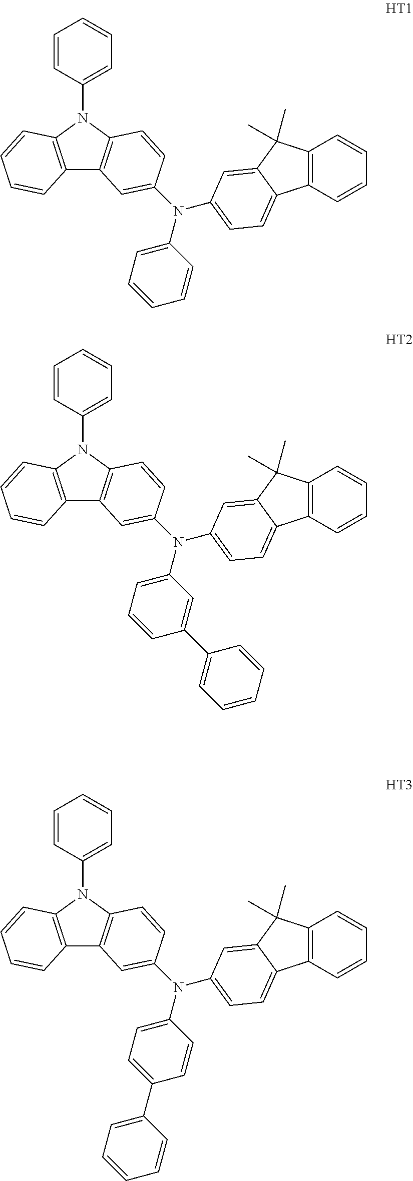 [SMILES: CC1(C)c2ccccc2-c2ccc(N(c3ccccc3)c3ccc4c(c3)c3ccccc3n4-c3ccccc3)cc21 ; CC1(C)c2ccccc2-c2ccc(N(c3cccc(-c4ccccc4)c3)c3ccc4c(c3)c3ccccc3n4-c3ccccc3)cc21 ; CC1(C)c2ccccc2-c2ccc(N(c3ccc(-c4ccccc4)cc3)c3ccc4c(c3)c3ccccc3n4-c3ccccc3)cc21]

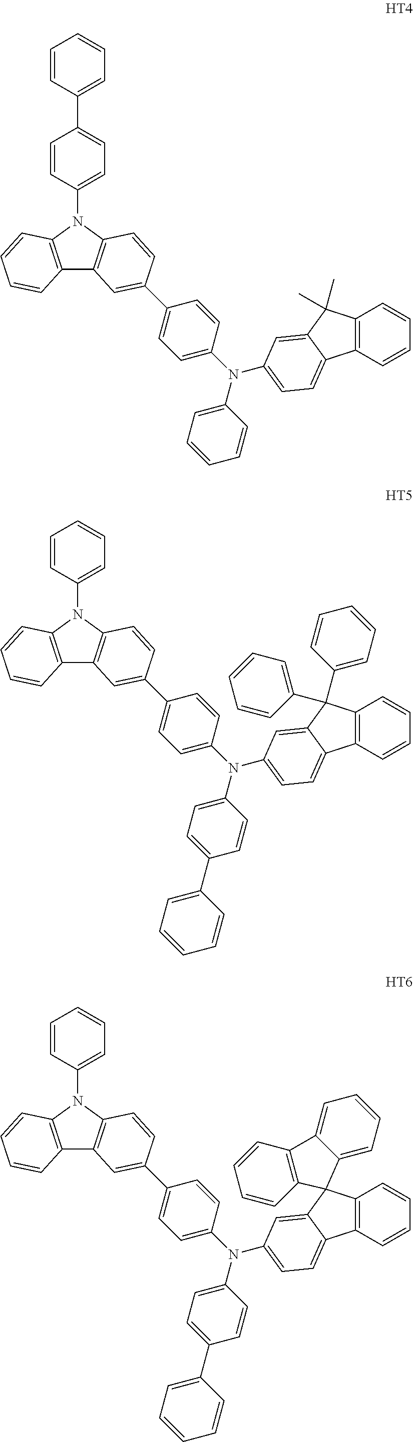 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

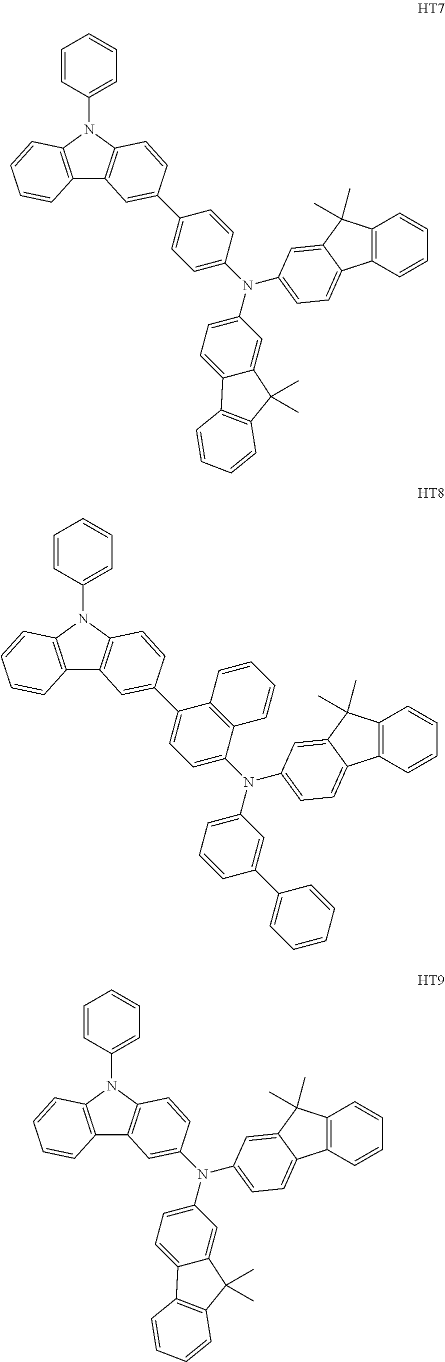 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21]

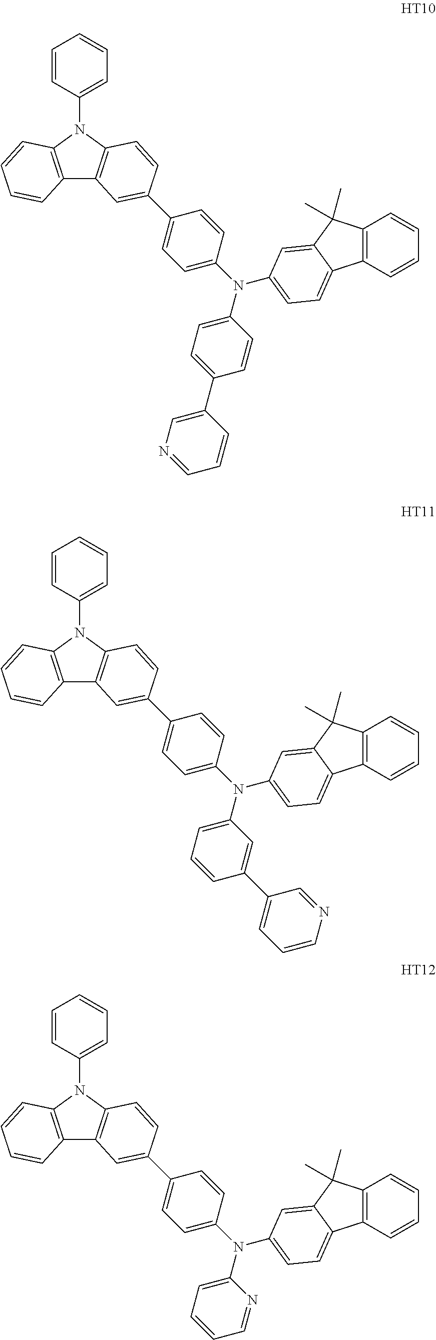 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

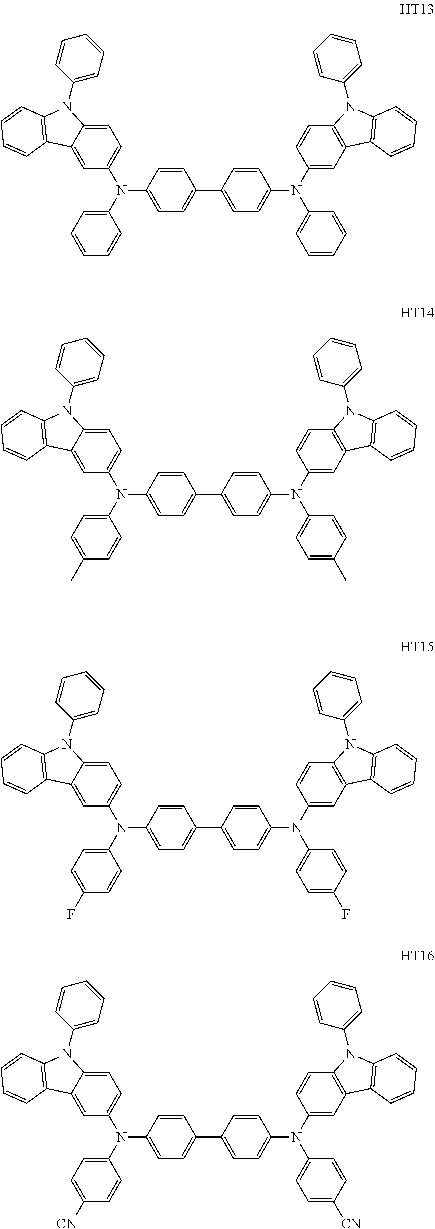

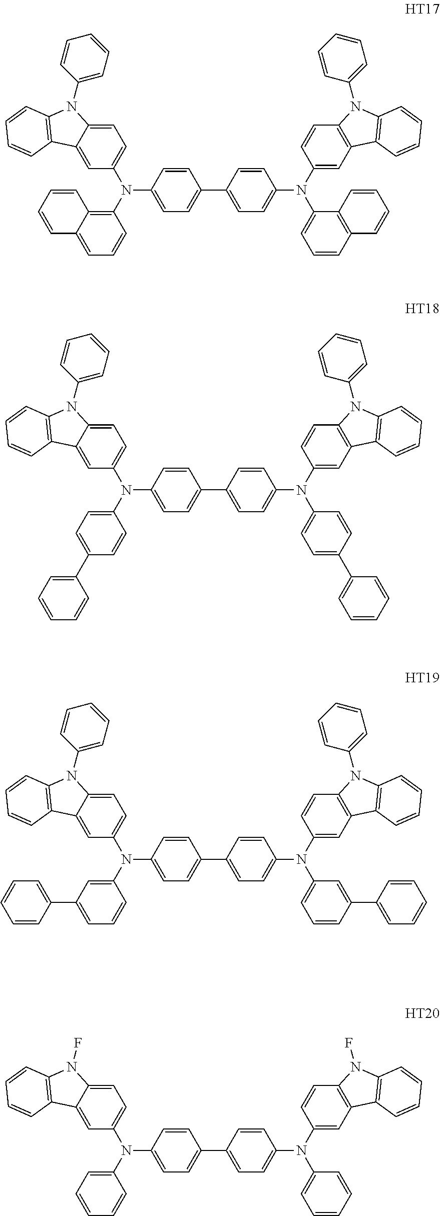 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fn1c2ccccc2c2cc(N(c3ccccc3)c3ccc(-c4ccc(N(c5ccccc5)c5ccc6c(c5)c5ccccc5n6F)cc4)cc3)ccc21]

A thickness of the hole transport region may range from about 100 Å to about 10,000 Å, for example, from about 100 Å to about 1,000 Å. When the hole transport region includes an HIL and an HTL, a thickness of the HIL may range from about 100 Å to about 10,000 Å, for example, from about 100 Å to about 1,000 Å, and a thickness of the HTL may range from about 50 Å to about 2,000 Å, for example, from about 100 Å to about 1,500 Å. When the thicknesses of the hole transport region, the HIL, and the HTL are within any of these ranges, satisfactory hole transport characteristics may be obtained without a substantial increase in driving voltage.

The hole transport region may further include, in addition to the materials described above, a charge-generation material for the improvement of conductive properties. The charge-generation material may be homogeneously or unhomogeneously dispersed in the hole transport region.

The charge-generation material may be, for example, a p-dopant. The p-dopant may be one selected from a quinone derivative, a metal oxide, and a cyano group-containing compound, but is not limited thereto. Non-limiting examples of the p-dopant include quinone derivatives (such as tetracyanoquinonedimethane (TCNQ) and/or 2,3,5,6-tetrafluoro-tetracyano-1,4-benzoquinonedimethane (F4-TCNQ)), metal oxides (such as tungsten oxide and/or molybdenum oxide), and Compound HT-D1.

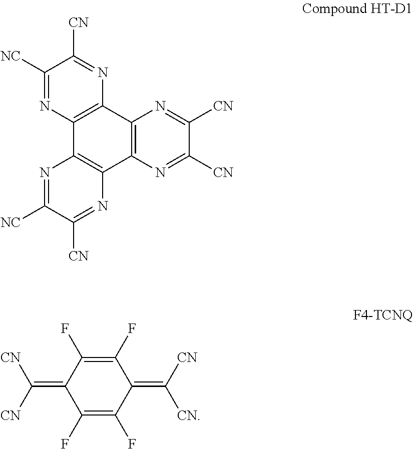

The hole transport region may further include, in addition to the HIL and the HTL as described above, at least one selected from a buffer layer and an EBL. The buffer layer may compensate for an optical resonance distance according to a wavelength of light emitted from the emission layer, thereby improving the light-emission efficiency of the formed organic light-emitting device. For use as a material included in the buffer layer, a material that may be included in the hole transport region may be used. The EBL may prevent or reduce the injection of electrons from the electron transport region.

The emission layer may be formed on the first electrode 110 or on the hole transport region by using one or more suitable methods such as vacuum deposition, spin coating, casting, an LB method, inkjet printing, laser printing, and/or LITI. When the emission layer is formed by vacuum deposition and/or spin coating, deposition and coating conditions for the emission layer may be similar to the deposition and coating conditions for the HIL.

When the organic light-emitting device 10 is a full color organic light-emitting device, the emission layer may be patterned into a red emission layer, a green emission layer, or a blue emission layer, according to a sub pixel. Alternatively, the emission layer may have a structure in which a red emission layer, a green emission layer, and a blue emission layer are stacked on one another or a structure in which a red-light emitting material, a green-light emitting material, and a blue-light emitting material are mixed with one another in a single layer, and thus may emit white light.

The emission layer may include a host and a dopant. The host may include the condensed cyclic compound represented by Formula 1.

The dopant may include at least one selected from a fluorescent dopant and a phosphorescent dopant.

The phosphorescent dopant may include an organometallic complex represented by Formula 401:

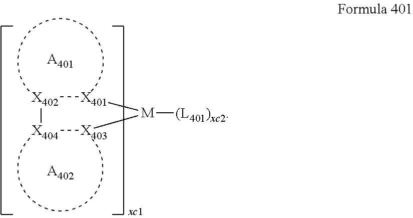

In Formula 401,

M may be selected from iridium (Ir), platinum (Pt), osmium (Os), titanium (Ti), zirconium (Zr), hafnium (Hf), europium (Eu), terbium (Tb), and thulium (Tm),

X₄₀₁ to X₄₀₄ may be each independently a nitrogen (N) or a carbon (C),

A₄₀₁ ring and A₄₀₂ ring may be each independently selected from a substituted or unsubstituted benzene, a substituted or unsubstituted naphthalene, a substituted or unsubstituted fluorene, a substituted or unsubstituted spiro-fluorene, a substituted or unsubstituted indene, a substituted or unsubstituted pyrrole, a substituted or unsubstituted thiophene, a substituted or unsubstituted furan, a substituted or unsubstituted imidazole, a substituted or unsubstituted pyrazole, a substituted or unsubstituted thiazole, a substituted or unsubstituted isothiazole, a substituted or unsubstituted oxazole, a substituted or unsubstituted isooxazole, a substituted or unsubstituted pyridine, a substituted or unsubstituted pyrazine, a substituted or unsubstituted pyrimidine, a substituted or unsubstituted pyridazine, a substituted or unsubstituted quinoline, a substituted or unsubstituted isoquinoline, a substituted or unsubstituted benzoquinoline, a substituted or unsubstituted quinoxaline, a substituted or unsubstituted quinazoline, a substituted or unsubstituted carbazole, a substituted or unsubstituted benzoimidazole, a substituted or unsubstituted benzofuran, a substituted or unsubstituted benzothiophene, a substituted or unsubstituted isobenzothiophene, a substituted or unsubstituted benzoxazole, a substituted or unsubstituted isobenzoxazole, a substituted or unsubstituted triazole, a substituted or unsubstituted oxadiazole, a substituted or unsubstituted triazine, a substituted or unsubstituted dibenzofuran, and a substituted or unsubstituted dibenzothiophene,

at least one substituent of the substituted benzene, substituted naphthalene, substituted fluorene, substituted spiro-fluorene, substituted indene, substituted pyrrole, substituted thiophene, substituted furan, substituted imidazole, substituted pyrazole, substituted thiazole, substituted isothiazole, substituted oxazole, substituted isoxazole, substituted pyridine, substituted pyrazine, substituted pyrimidine, substituted pyridazine, substituted quinoline, substituted isoquinoline, substituted benzoquinoline, substituted quinoxaline, substituted quinazoline, substituted carbazole, substituted benzoimidazole, substituted benzofuran, substituted benzothiophene, substituted isobenzothiophene, substituted benzoxazole, substituted isobenzoxazole, substituted triazole, substituted oxadiazole, substituted triazine, substituted dibenzofuran, and substituted dibenzothiophene may be selected from:

deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₄₀₁)(Q₄₀₂), —Si(Q₄₀₃)(Q₄₀₄)(Q₄₀₅), and —B(Q₄₀₆)(Q₄₀₇);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₄₁₁)(Q₄₁₂), —Si(Q₄₁₃)(Q₄₁₄)(Q₄₁₅), and —B(Q₄₁₆)(Q₄₁₇); and

—N(Q₄₂₁)(Q₄₂₂), —Si(Q₄₂₃)(Q₄₂₄)(Q₄₂₅), and —B(Q₄₂₆)(Q₄₂₇),

L₄₀₁ may be an organic ligand,

xc1 may be 1, 2, or 3, and

xc2 may be 0, 1, 2, or 3.

Each of descriptions of Q₄₀₁ to Q₄₀₇, Q₄₁₁ to Q₄₁₇, and Q₄₂₁ to Q₄₂₇ may be the same as provided herein in connection with Q₁.

L₄₀₁ may be a monovalent, divalent, or trivalent organic ligand. For example, L₄₀₁ may be selected from a halogen ligand (for example, Cl and/or F), a diketone ligand (for example, acetylacetonate, 1,3-diphenyl-1,3-propanedionate, 2,2,6,6-tetramethyl-3,5-heptanedionate, and/or hexafluoroacetonate), a carboxylic acid ligand (for example, picolinate, dimethyl-3-pyrazolecarboxylate, and/or benzoate), a carbon monoxide ligand, an isonitrile ligand, a cyano ligand, and a phosphorus ligand (for example, phosphine and/or phosphite), but is not limited thereto.

When A₄₀₁ in Formula 401 has two or more substituents, the substituents of A₄₀₁ may bind to each other to form a saturated or unsaturated ring.

When A₄₀₂ in Formula 401 has two or more substituents, the substituents of A₄₀₂ may bind to each other to form a saturated or unsaturated ring.

When xc1 in Formula 401 is two or more, a plurality of ligands in Formula 401

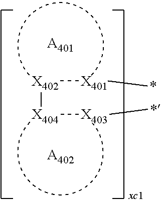

may be identical to or different from each other. When xc1 in Formula 401 is two or more, A₄₀₁ and/or A₄₀₂ of one ligand may be respectively connected to A₄₀₁ and/or A₄₀₂ of other neighboring ligands directly (e.g., via a bond such as a single bond) or with a linker such as a linking group (for example, a C₁-C₅ alkylene group, —N(R′)— (where R′ may be a C₁-C₁₀ alkyl group or a C₆-C₂₀ aryl group), and/or —C(═O)—) therebetween.

The phosphorescent dopant may include at least one selected from Compounds PD1 to PD74, but is not limited thereto:

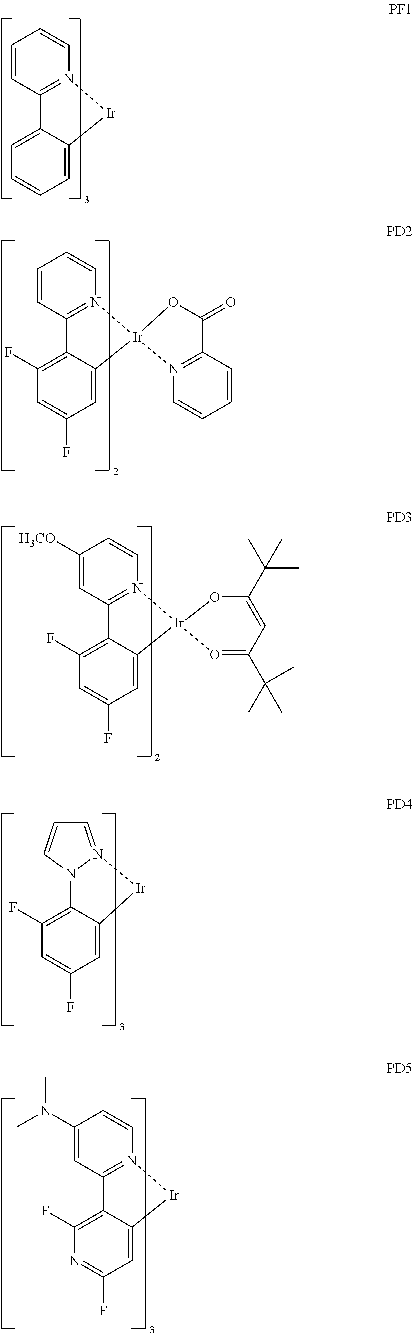

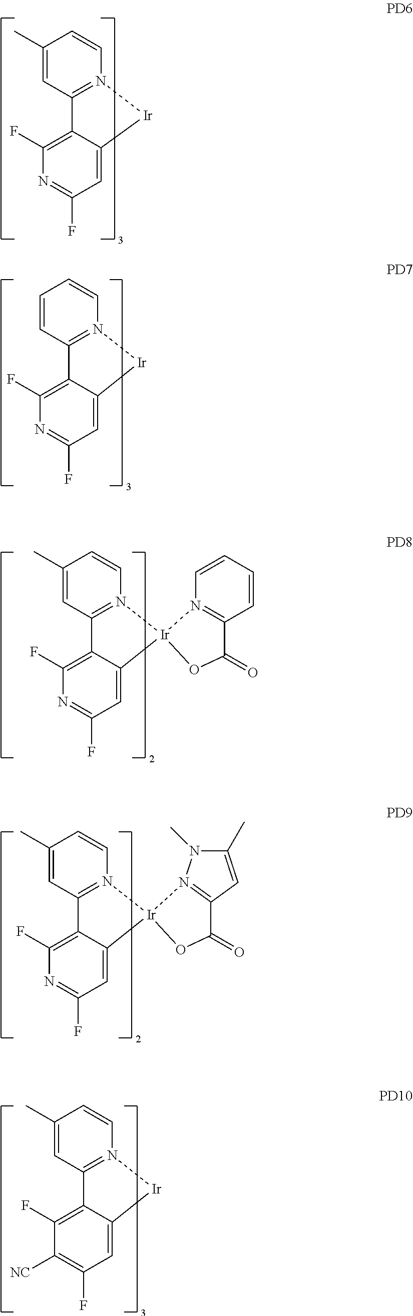

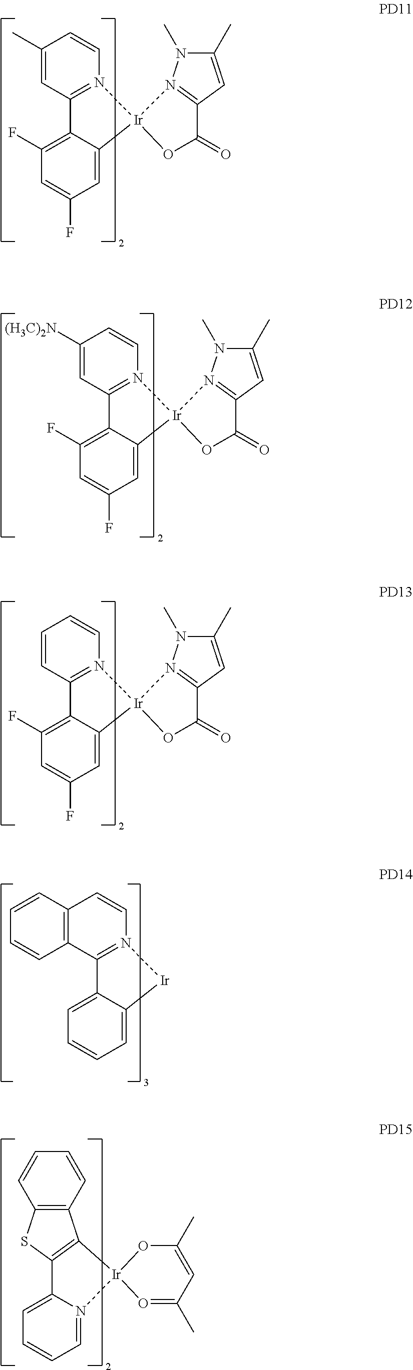

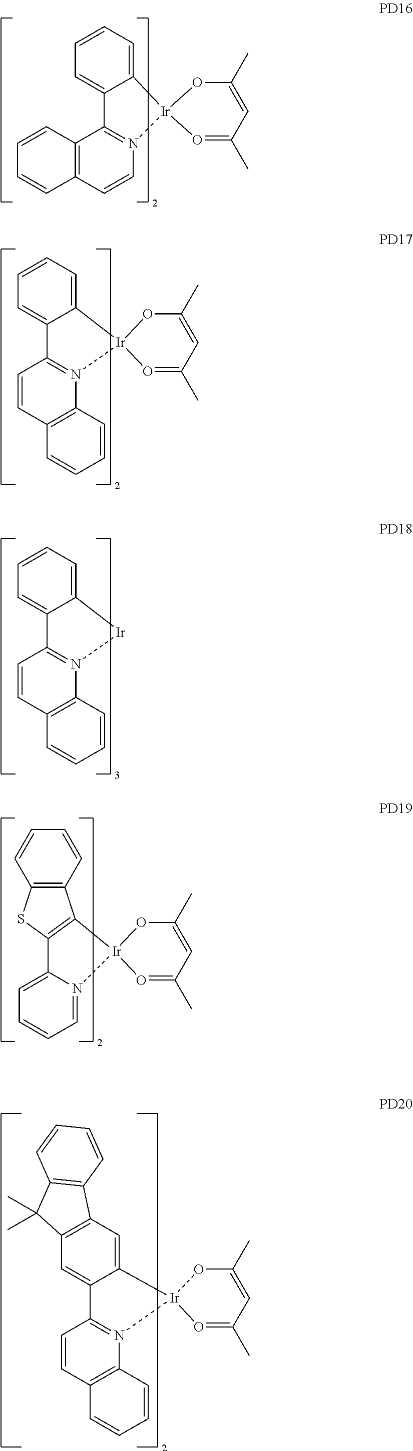

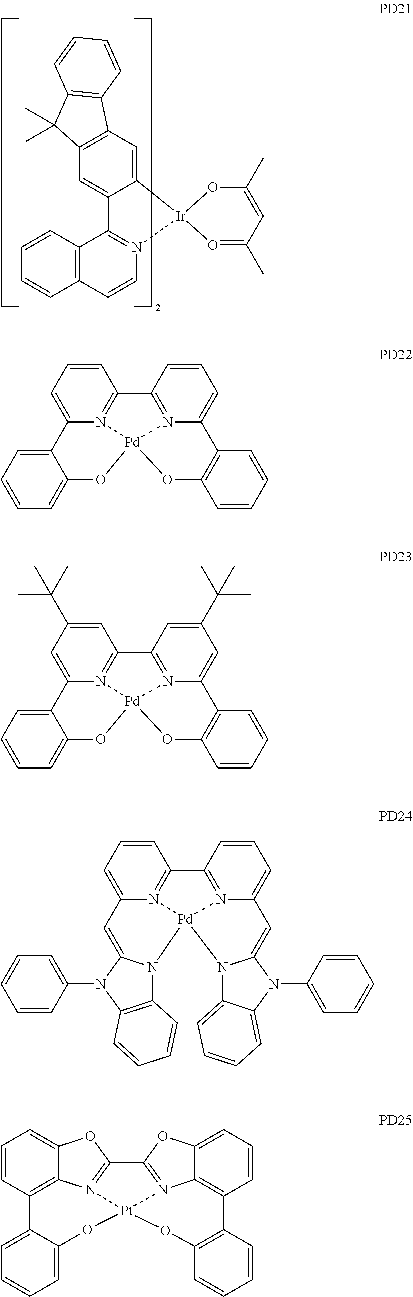

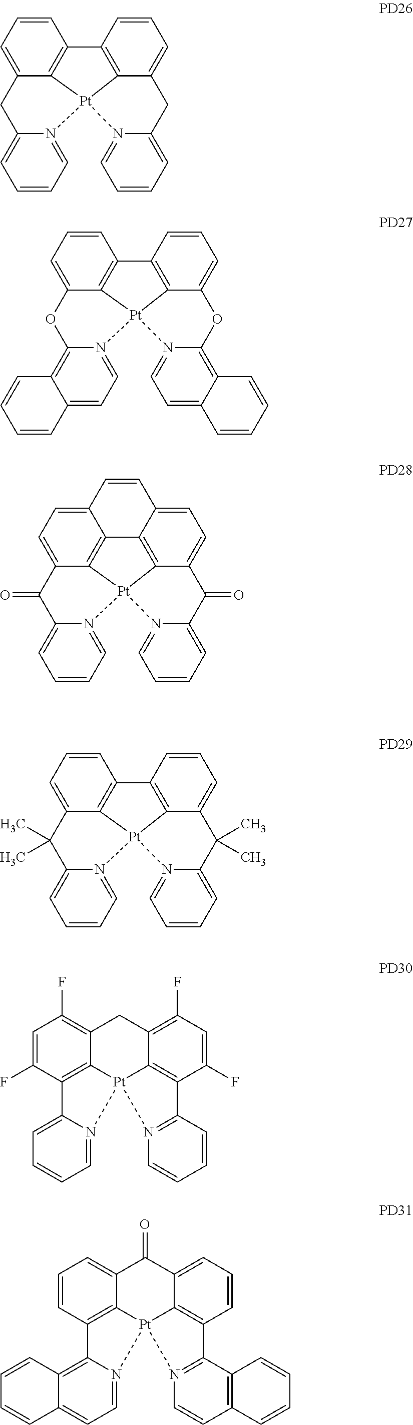

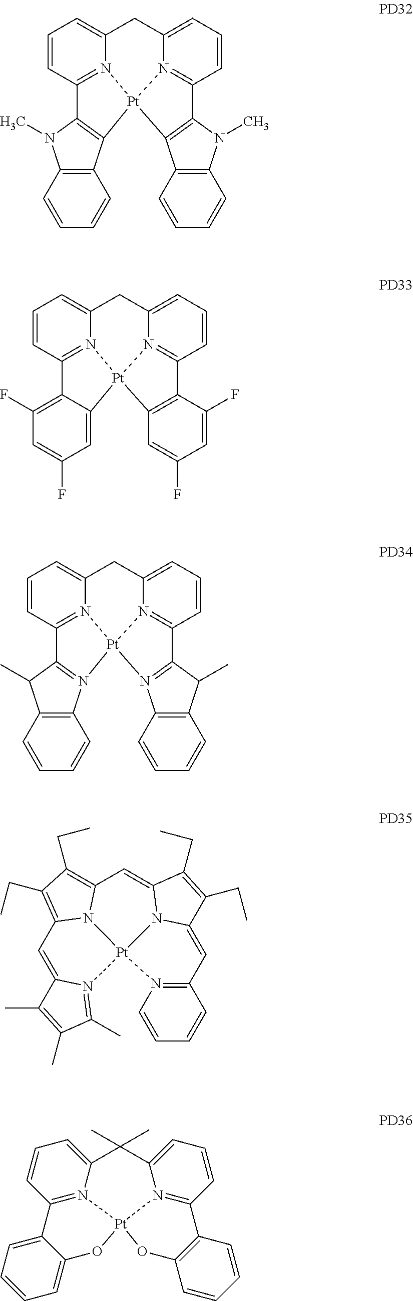

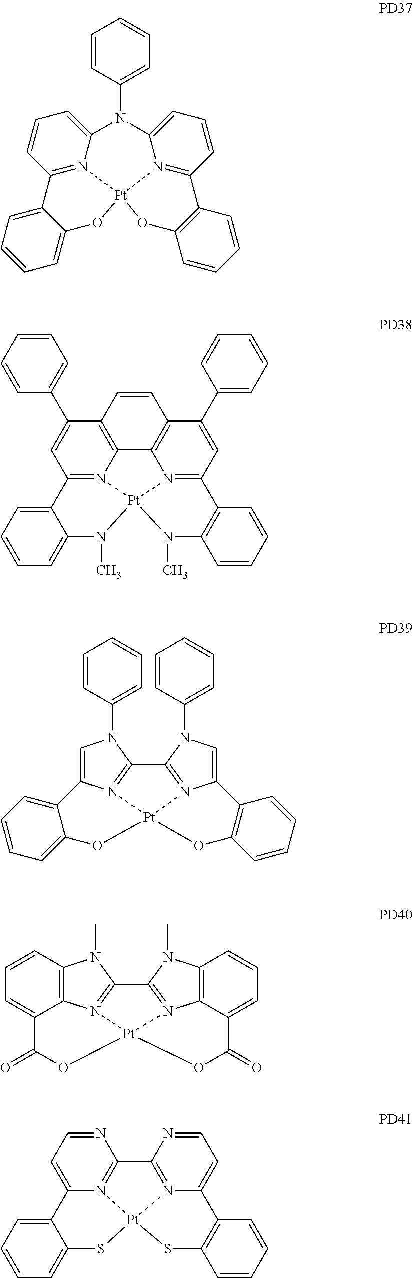

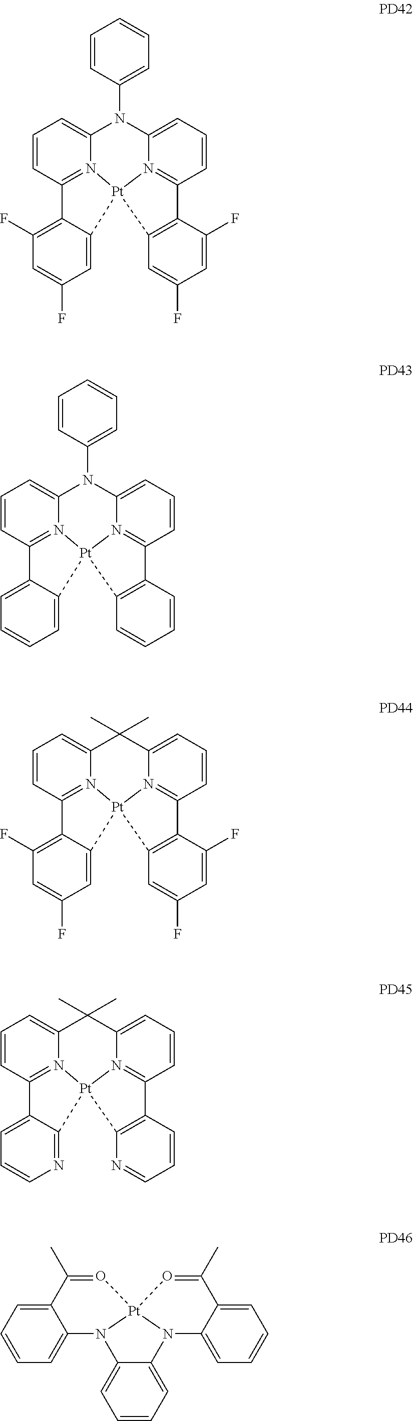

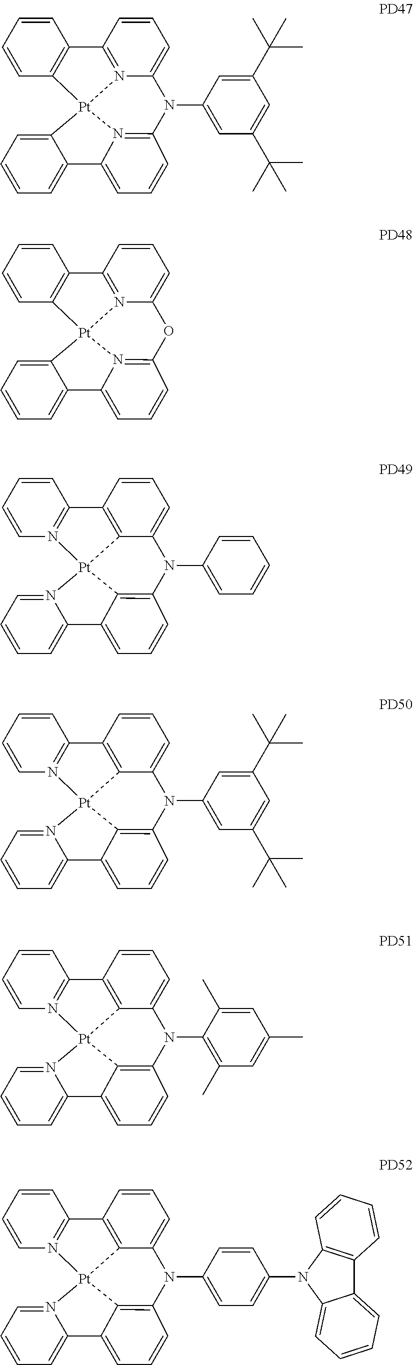

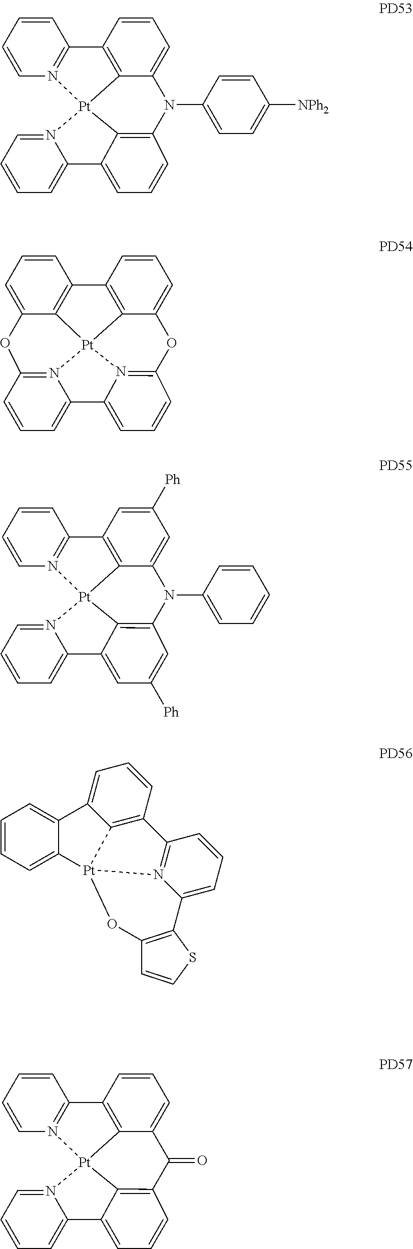

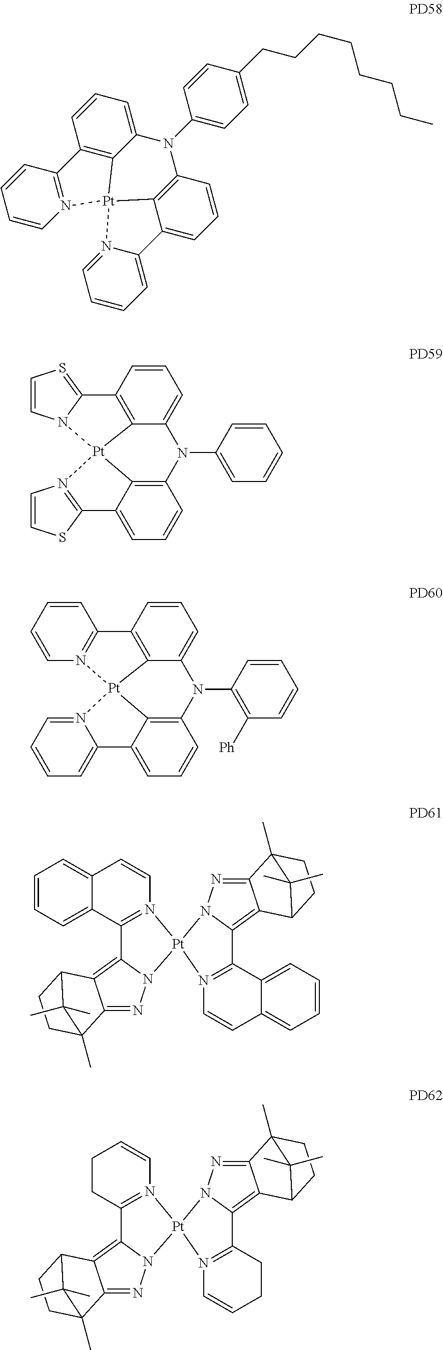

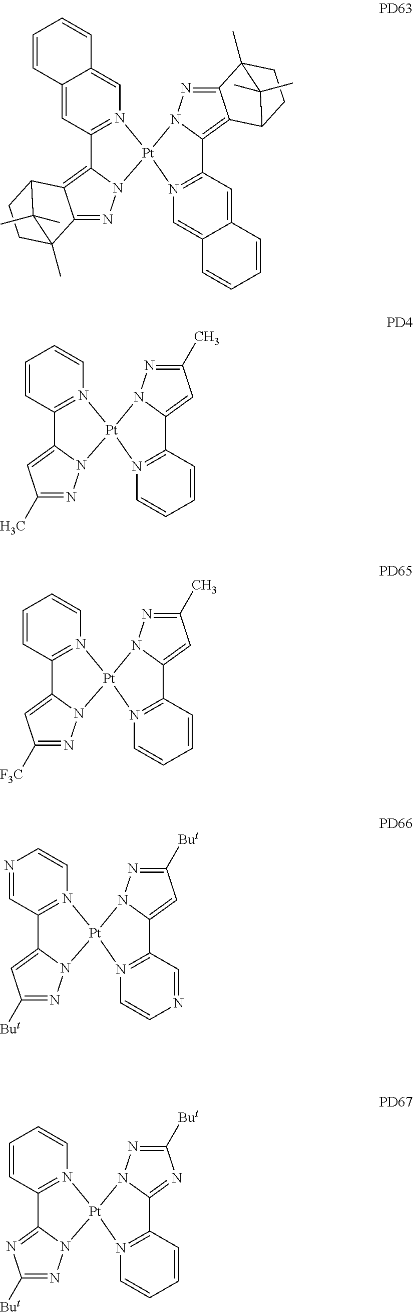

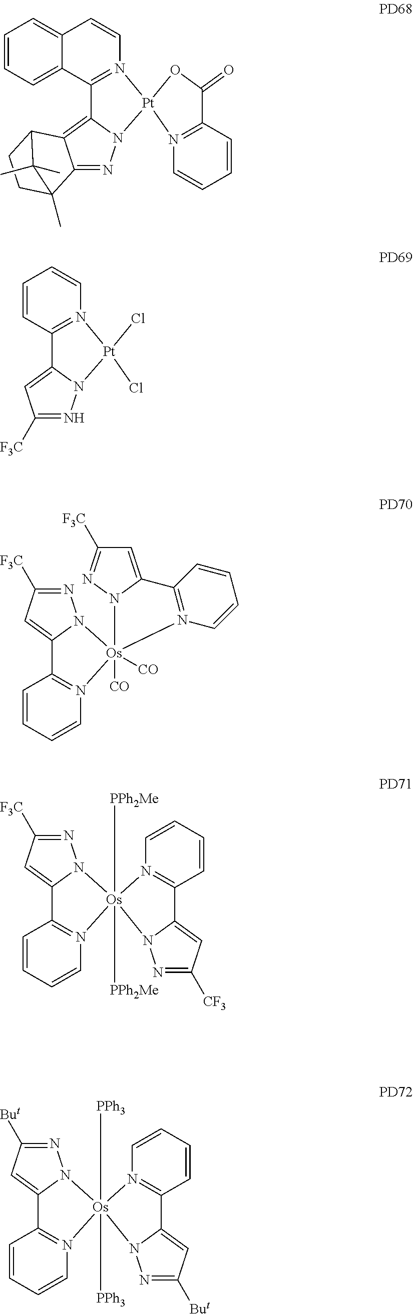

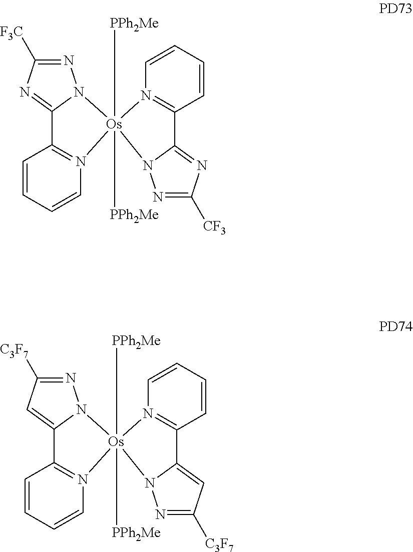

In Compounds PD1 to PD74, “Me” may refer to a methyl group, “Ph” may refer to a phenyl group, and “Bu^{t}” may refer to a tert-butyl group.

In some embodiments, the phosphorescent dopant may include PtOEP:

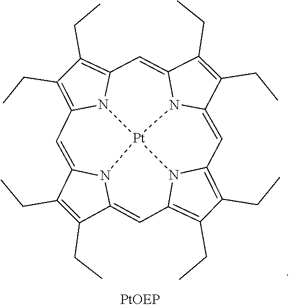

The fluorescent dopant may include at least one selected from DPVBi, DPAVBi, TBPe, DCM, DCJTB, Coumarin 6, and C545T.

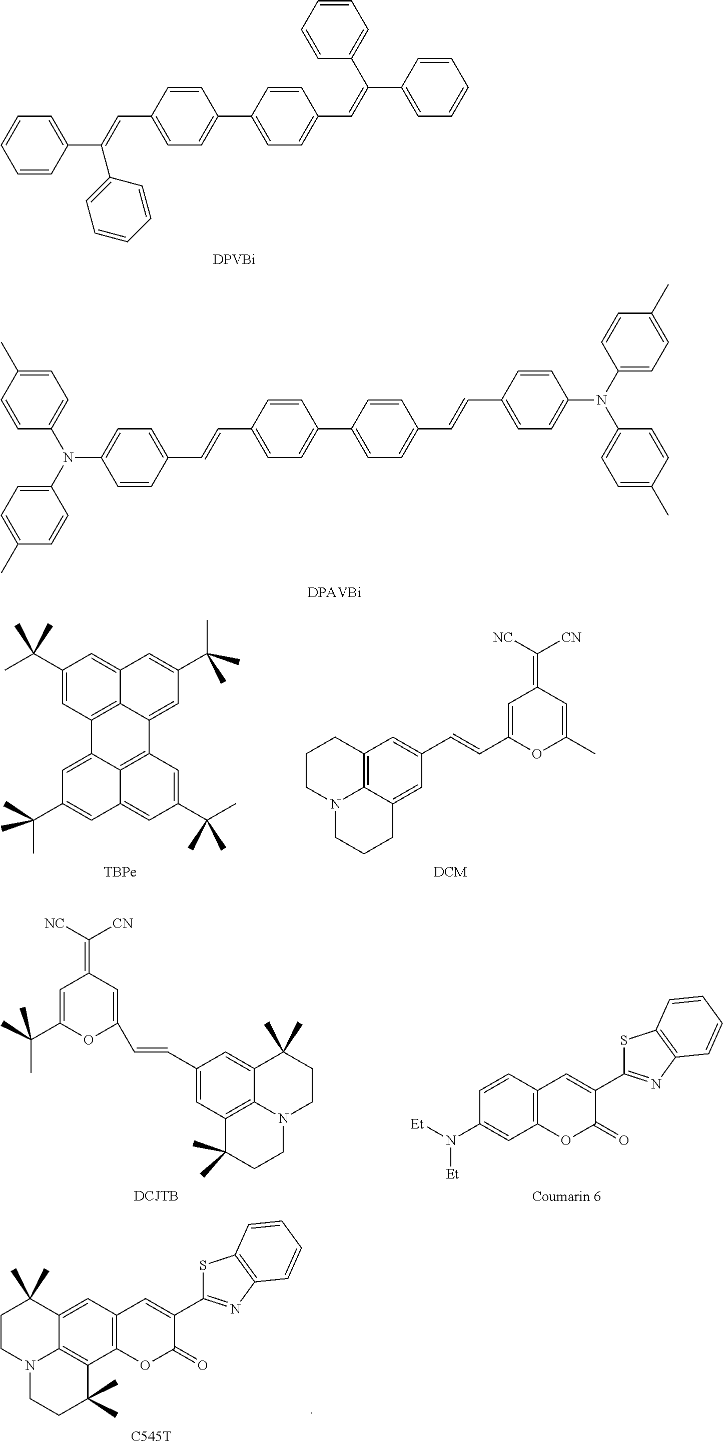

In some embodiments, the fluorescent dopant may include a compound represented by Formula 501:

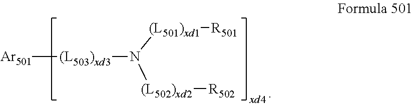

In Formula 501,

Ar₅₀₁ may be selected from a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene; and

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₅₀₁)(Q₅₀₂)(Q₅₀₃) (where Q₅₀₁ to Q₅₀₃ may be each independently selected from hydrogen, a C₁-C₆₀ alkyl group, a C₂-C₆₀ alkenyl group, a C₆-C₆₀ aryl group, and a C₁-C₆₀ heteroaryl group),

each of descriptions of L₅₀₁ to L₅₀₃ may be the same as provided herein in connection with L₂₀₁,

R₅₀₁ and R₅₀₂ may be each independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, and a dibenzothiophenyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group,

xd1 to xd3 may be each independently selected from 0, 1, 2, and 3, and

xd4 may be selected from 1, 2, 3, and 4.

For example, the fluorescent dopant may be represented by at least one selected from Compounds FD1 to FD9:

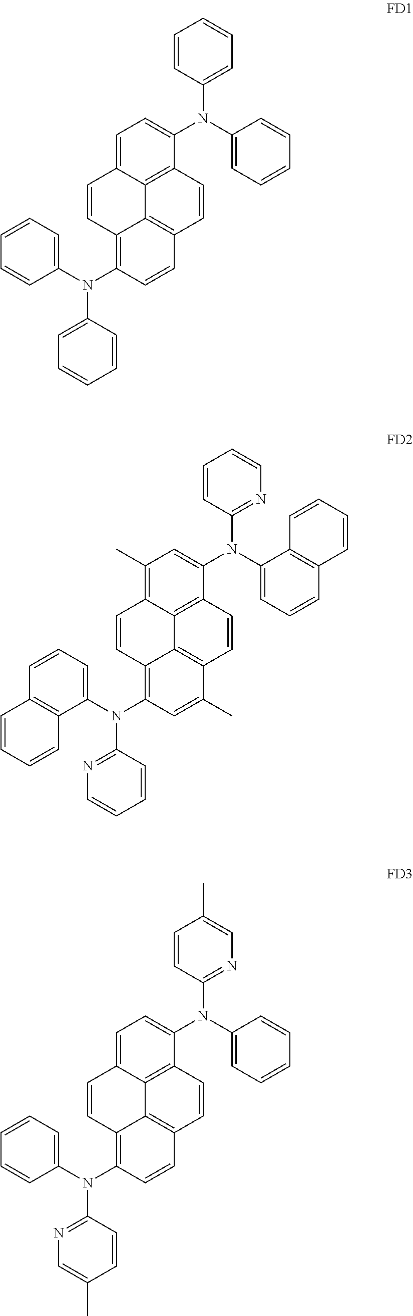 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

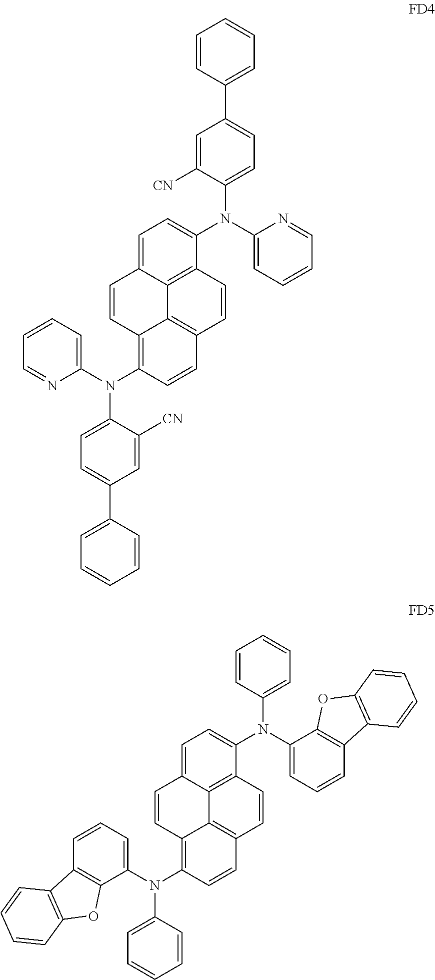

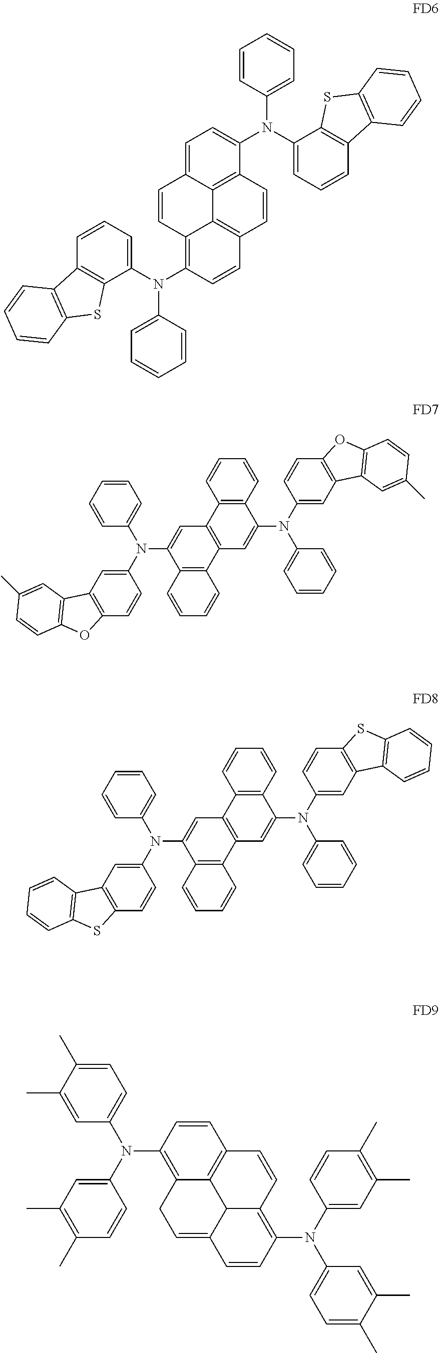

An amount of the dopant in the emission layer, may range from about 0.01 to about 15 parts by weight based on about 100 parts by weight of the host, but is not limited thereto.

A thickness of the emission layer may range from about 100 Å to about 1,000 Å, for example, from about 200 Å to about 600 Å. When the thickness of the emission layer is within any of these ranges, excellent light-emission characteristics may be obtained without a substantial increase in driving voltage.

An electron transport region may be positioned on the emission layer.

The electron transport region may include at least one selected from an HBL, an ETL, and an EIL, but is not limited thereto.

For example, the electron transport region may have a structure of ETL/EIL or a structure of HBL/ETL/EIL, where the layers of each structure are sequentially stacked from the emission layer in this stated order. However, the structure of the electron transport region is not limited thereto.

In some embodiments, the organic layer 150 of the organic light-emitting device 10 may include an electron transport region between the emission layer and the second electrode 190.

When the electron transport region includes an HBL, the HBL may be formed on the emission layer by using one or more suitable methods such as vacuum deposition, spin coating, casting, an LB method, inkjet printing, laser printing, and/or LITI. When the HBL is formed by vacuum deposition and/or spin coating, deposition and coating conditions for the HBL may be similar to the deposition and coating conditions for the HIL.

The HBL may include, for example, at least one selected from BCP and Bphen, but is not limited thereto.

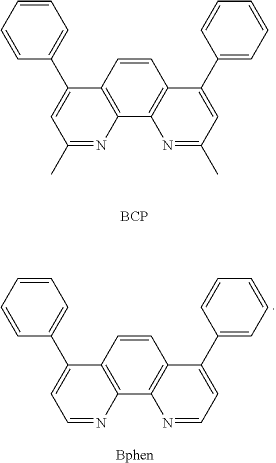

A thickness of the HBL may range from about 20 Å to about 1,000 Å, for example, from about 30 Å to about 300 Å. When the thickness of the HBL is within any of these ranges, excellent hole blocking characteristics may be obtained without a substantial increase in driving voltage.

The electron transport region may include an ETL. The ETL may be formed on the emission layer or on the HBL by using one or more suitable methods such as vacuum deposition, spin coating, casting, an LB method, inkjet printing, laser printing, and/or LITI. When the ETL is formed by vacuum deposition and/or spin coating, deposition and coating conditions for the ETL may be similar to the deposition and coating conditions for the HIL.

The ETL may include at least one selected from a compound represented by Formula 601 and a compound represented by Formula 602.

Ar₆₀₁-[(L₆₀₁)ₓₑ₁-E₆₀₁]ₓₑ₂. Formula 601

In Formula 601,

Ar₆₀₁ may be selected from:

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene; and

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₃₀₁)(Q₃₀₂)(Q₃₀₃) (where Q₃₀₁ to Q₃₀₃ may be each independently selected from hydrogen, a C₁-C₆₀ alkyl group, a C₂-C₆₀ alkenyl group, a C₆-C₆₀ aryl group, and a C₁-C₆₀ heteroaryl group),

description of L₆₀₁ may be the same as provided herein in connection with L₂₀₁,

E₆₀₁ may be selected from:

a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and

a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group,

xe1 may be selected from 0, 1, 2, and 3, and

xe2 may be selected from 1, 2, 3, and 4.

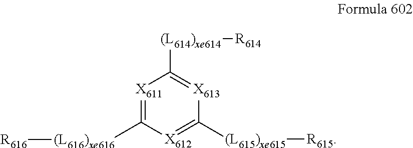

In Formula 602,

X₆₁₁ may be N or C-(L₆₁₁)ₓₑ₆₁₁-R₆₁₁, X₆₁₂ may be N or C-(L₆₁₂)ₓₑ₆₁₂-R₆₁₂, X₆₁₃ may be N or C-(L₆₁₃)ₓₑ₆₁₃-R₆₁₃, and at least one selected from X₆₁₁ to X₆₁₃ may be N,

description of each of L₆₁₁ to L₆₁₆ may be the same as provided herein in connection with L₁,

R₆₁₁ to R₆₁₆ may be each independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an azulenyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, and

xe611 to xe616 may be each independently selected from 0, 1, 2, and 3.

The compound represented by Formula 601 and the compound represented by Formula 602 may be each independently selected from Compounds ET1 to ET15:

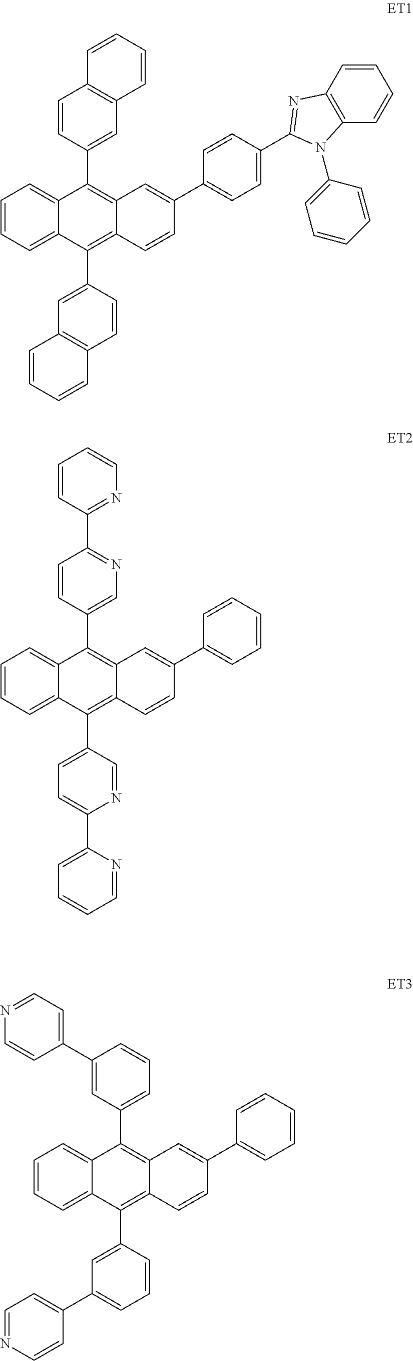 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

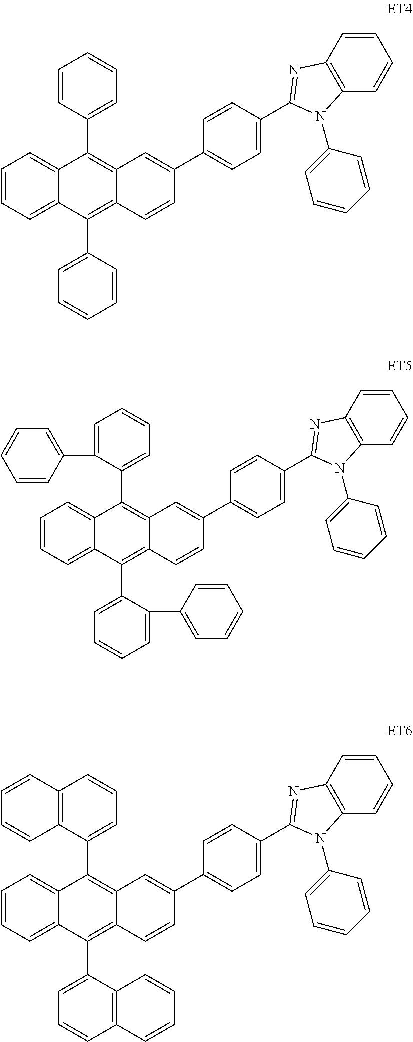 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

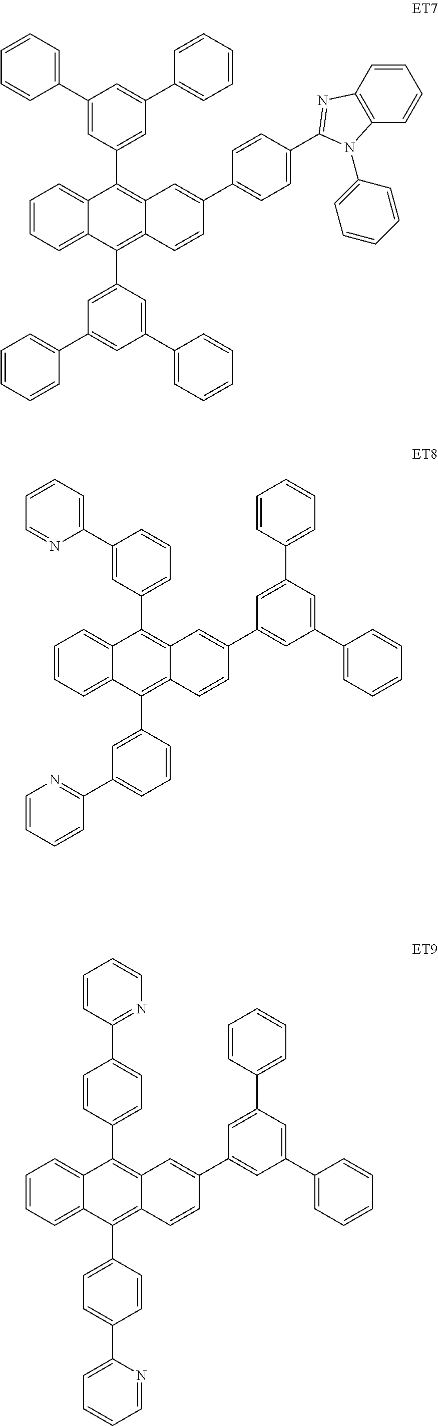 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

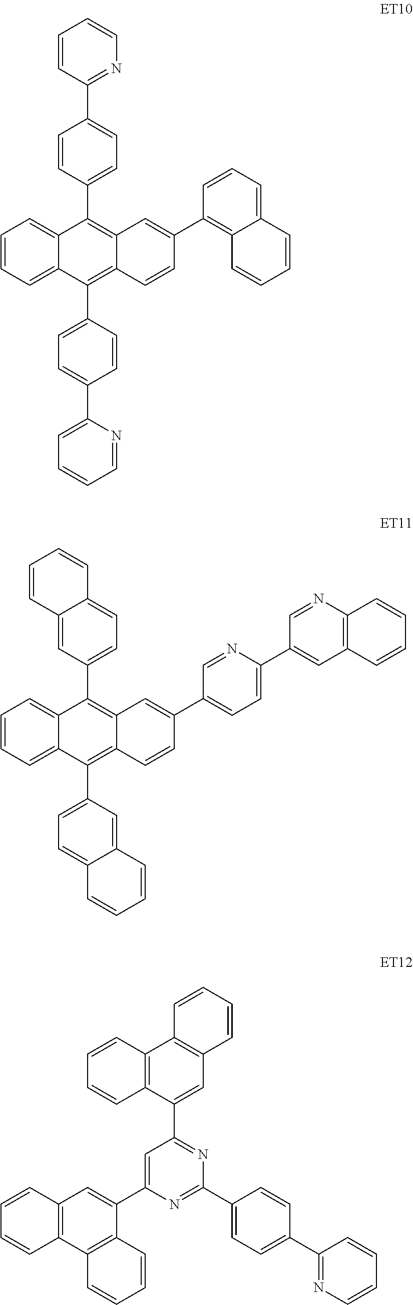 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-c2ccc(-c3nc(-c4cc5ccccc5c5ccccc45)cc(-c4cc5ccccc5c5ccccc45)n3)cc2)nc1]

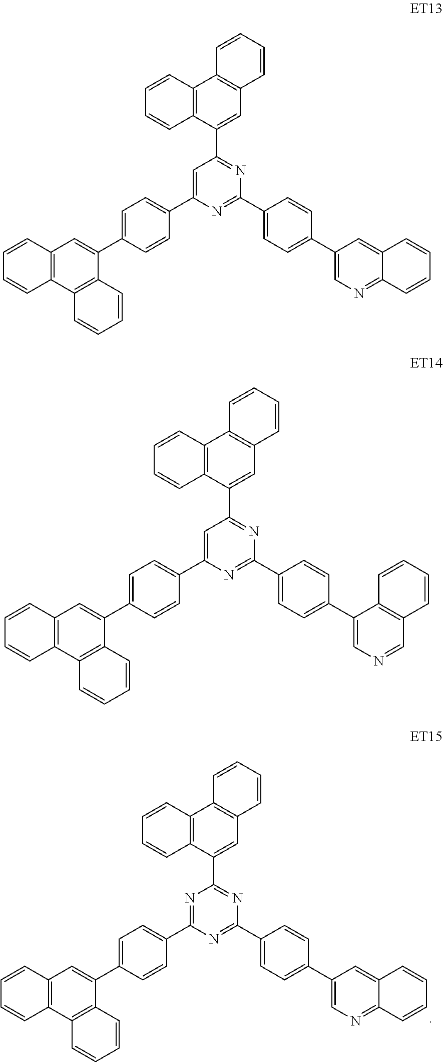 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5ccc(-c6cc7ccccc7c7ccccc67)cc5)cc(-c5cc6ccccc6c6ccccc56)n4)cc3)cncc2c1 ; c1ccc2ncc(-c3ccc(-c4nc(-c5ccc(-c6cc7ccccc7c7ccccc67)cc5)nc(-c5cc6ccccc6c6ccccc56)n4)cc3)cc2c1]

In some embodiments, the ETL may include at least one selected from BCP, Bphen, Alq₃, Balq, TAZ, and NTAZ.

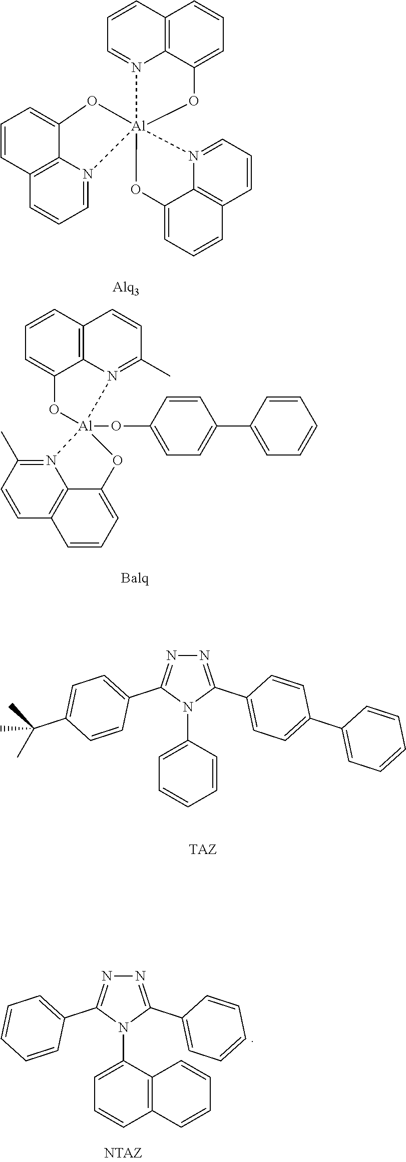

A thickness of the ETL may range from about 100 Å to about 1,000 Å, for example, from about 150 Å to about 500 Å. When the thickness of the ETL is within any of these ranges, satisfactory electron transport characteristics may be obtained without a substantial increase in driving voltage.

The ETL may further include, in addition to the materials described above, a metal-containing material.

The metal-containing material may include a Li complex. The Li complex may include, for example, Compound ET-D1 (lithium quinolate, LiQ) and/or ET-D2.

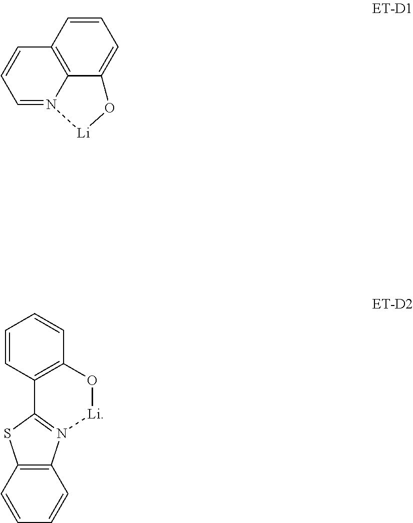

The electron transport region may include an EIL which facilitates the injection of electrons from the second electrode 190.

The EIL may be formed on the ETL by using one or more suitable methods such as vacuum deposition, spin coating, casting, an LB method, inkjet printing, laser printing, and/or LITI. When the EIL is formed by vacuum deposition and/or spin coating, deposition and coating conditions for the EIL may be similar to the deposition and coating conditions for the HIL.

The EIL may include at least one selected from LiF, NaCl, CsF, Li₂O, BaO, and LiQ.

A thickness of the EIL may range from about 1 Å to about 100 Å, for example, from about 3 Å to about 90 Å. When the thickness of the EIL is within any of these ranges, satisfactory electron injection characteristics may be obtained without a substantial increase in driving voltage.

The second electrode 190 may be positioned on the organic layer 150 having the structure according to embodiments of the present disclosure. The second electrode 190 may be a cathode that is an electron injection electrode. When the second electrode 190 is a cathode, a material for forming the second electrode 190 may be a material having a low work function, for example, a metal, an alloy, an electrically conductive compound, or a mixture thereof. Non-limiting examples of the material for forming the second electrode 190 include lithium (Li), magnesium (Mg), aluminum (Al), aluminum-lithium (Al—Li), calcium (Ca), magnesium-indium (Mg—In), and magnesium-silver (Mg—Ag). In some embodiments, ITO and/or IZO may be used as the material for forming the second electrode 190. The second electrode 190 may be a semi-transmissive electrode or a transmissive electrode.

Although the organic light-emitting device has been described above with reference to the drawing, the organic light-emitting device of embodiments of the present disclosure is not limited thereto.

A C₁-C₆₀ alkyl group used herein may refer to a linear or branched aliphatic hydrocarbon monovalent group having 1 to 60 carbon atoms, and non-limiting examples thereof include a methyl group, an ethyl group, a propyl group, an isobutyl group, a sec-butyl group, a tert-butyl group, a pentyl group, an iso-amyl group, and a hexyl group. A C₁-C₆₀ alkylene group used herein may refer to a divalent group having the same structure as the C₁-C₆₀ alkyl group.

A C₁-C₆₀ alkoxy group used herein may refer to a monovalent group represented by —OA₁₀₁ (where A₁₀₁ is the C₁-C₆₀ alkyl group), and non-limiting examples thereof include a methoxy group, an ethoxy group, and an isopropyloxy group.

A C₂-C₆₀ alkenyl group used herein may refer to a hydrocarbon group having at least one carbon double bond at one or more positions along a carbon chain of the C₂-C₆₀ alkyl group (e.g., in the middle or at either terminal end of the C₂-C₆₀ alkyl group), and non-limiting examples thereof include an ethenyl group, a propenyl group, and a butenyl group. A C₂-C₆₀ alkenylene group used herein may refer to a divalent group having the same structure as the C₂-C₆₀ alkenyl group.

A C₂-C₆₀ alkynyl group used herein may refer to a hydrocarbon group having at least one carbon triple bond at one or more positions along a carbon chain of the C₂-C₆₀ alkyl group (e.g., in the middle or at either terminal end of the C₂-C₆₀ alkyl group), and non-limiting examples thereof include an ethynyl group and a propynyl group. A C₂-C₆₀ alkynylene group used herein may refer to a divalent group having the same structure as the C₂-C₆₀ alkynyl group.

A C₃-C₁₀ cycloalkyl group used herein may refer to a monovalent saturated hydrocarbon monocyclic group having 3 to 10 carbon atoms, and non-limiting examples thereof include a cyclopropyl group, a cyclobutyl group, a cyclopentyl group, a cyclohexyl group, and a cycloheptyl group. A C₃-C₁₀ cycloalkylene group used herein may refer to a divalent group having the same structure as the C₃-C₁₀ cycloalkyl group.

A C₁-C₁₀ heterocycloalkyl group used herein may refer to a monovalent monocyclic group having at least one heteroatom selected from N, O, Si, P, and S as a ring-forming atom and 1 to 10 carbon atoms, and non-limiting examples thereof include a tetrahydrofuranyl group and a tetrahydrothiophenyl group. A C₁-C₁₀ heterocycloalkylene group used herein may refer to a divalent group having the same structure as the C₁-C₁₀ heterocycloalkyl group.

A C₃-C₁₀ cycloalkenyl group used herein may refer to a monovalent monocyclic group that has 3 to 10 carbon atoms and at least one double bond in the ring thereof and does not have aromaticity, and non-limiting examples thereof include a cyclopentenyl group, a cyclohexenyl group, and a cycloheptenyl group. A C₃-C₁₀ cycloalkenylene group used herein may refer to a divalent group having the same structure as the C₃-C₁₀ cycloalkenyl group.

A C₁-C₁₀ heterocycloalkenyl group used herein may refer to a monovalent monocyclic group that has at least one heteroatom selected from N, O, Si, P, and S as a ring-forming atom, 1 to 10 carbon atoms, and at least one double bond in its ring. Non-limiting examples of the C₁-C₁₀ heterocycloalkenyl group include a 2,3-hydrofuranyl group and a 2,3-hydrothiophenyl group. A C₁-C₁₀ heterocycloalkenylene group used herein may refer to a divalent group having the same structure as the C₁-C₁₀ heterocycloalkenyl group.

A C₆-C₆₀ aryl group used herein may refer to a monovalent group having a carbocyclic aromatic system having 6 to 60 carbon atoms, and a C₆-C₆₀ arylene group used herein may refer to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and/or the C₆-C₆₀ arylene group include two or more rings, the rings may be fused to each other.

A C₁-C₆₀ heteroaryl group used herein may refer to a monovalent group having a carbocyclic aromatic system that has at least one heteroatom selected from N, O, Si, P, and S as a ring-forming atom and 1 to 60 carbon atoms. A C₁-C₆₀ heteroarylene group used herein may refer to a divalent group having a carbocyclic aromatic system that has at least one heteroatom selected from N, O, P, and S as a ring-forming atom and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and/or the C₁-C₆₀ heteroarylene group include two or more rings, the rings may be fused to each other.

A C₆-C₆₀ aryloxy group used herein may refer to a monovalent group represented by —OA₁₀₂ (where A₁₀₂ is the C₆-C₆₀ aryl group), and a C₆-C₆₀ arylthio group used herein may refer to a monovalent group represented by —SA₁₀₃ (where A₁₀₃ is the C₆-C₆₀ aryl group).

A monovalent non-aromatic condensed polycyclic group used herein may refer to a monovalent group that has two or more rings condensed (e.g., fused) to each other, only carbon atoms as ring-forming atoms (for example, 8 to 60 carbon atoms), and does not have overall aromaticity in the entire molecular structure. A non-limiting example of the monovalent non-aromatic condensed polycyclic group is a fluorenyl group. A divalent non-aromatic condensed polycyclic group used herein may refer to a divalent group having the same structure as the monovalent non-aromatic condensed polycyclic group.

A monovalent non-aromatic condensed heteropolycyclic group used herein may refer to a monovalent group that has two or more rings condensed (e.g., fused) to each other, has at least one heteroatom selected from N, O, Si, P, and S, other than carbon atoms (for example, 1 to 60 carbon atoms), as ring-forming atoms, and does not have overall aromacity in the entire molecular structure. A non-limiting example of the monovalent non-aromatic condensed heteropolycyclic group is a carbazolyl group. A divalent non-aromatic condensed heteropolycyclic group used herein may refer to a divalent group having the same structure as the monovalent non-aromatic condensed heteropolycyclic group.

At least one substituent of the substituted C₃-C₁₀ cycloalkylene group, substituted C₁-C₁₀ heterocycloalkylene group, substituted C₃-C₁₀ cycloalkenylene group, substituted C₁-C₁₀ heterocycloalkenylene group, substituted C₆-C₆₀ arylene group, substituted C₁-C₆₀ heteroarylene group, substituted divalent non-aromatic condensed polycyclic group, substituted divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group may be selected from:

deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₁₁)(Q₁₂)(Q₁₃);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₂₁)(Q₂₂)(Q₂₃); and

—Si(Q₃₁)(Q₃₂)(Q₃₃),

where Q₁₁ to Q₁₃, Q₂₁ to Q₂₃, and Q₃₁ to Q₃₃ may be each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

The term “Ph” used herein refers to a phenyl group, the term “Me” used herein refers to a methyl group, the term “Et” used herein refers to an ethyl group, and the term “ter-Bu” or “Bu^{t}” used herein refers to a tert-butyl group.

Hereinafter, an organic light-emitting device according to one or more embodiments of the present disclosure will be described in more detail with reference to Synthesis Examples and Examples. The phrase “B was used instead of A” used in describing Synthesis Examples below may refer to a molar equivalent of A being identical to a molar equivalent of B.

### EXAMPLES

### Synthesis Example 1

### Synthesis of Compound 1

### Synthesis of Intermediate 1-1

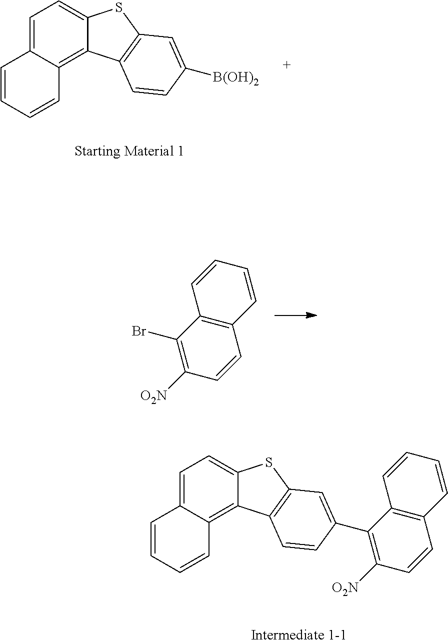 [SMILES: OB(O)c1ccc2c(c1)sc1ccc3ccccc3c12 ; O=[N+]([O-])c1ccc2ccccc2c1Br ; O=[N+]([O-])c1ccc2ccccc2c1-c1ccc2c(c1)sc1ccc3ccccc3c12]

5 g (1 eq) of Starting Material 1, 5.08 g (1.2 eq) of 1-bromo-2-nitronaphthalene, and 0.9 g (0.03 eq) of Pd(PPh₃)₄ were put in a flask and dissolved in 120 ml of toluene while being packed with nitrogen. Next, 40 ml of an aqueous solution in which 10 g of sodium carbonate was dissolved was added thereto, and then, the result was reflux-stirred for 12 hours. After the reaction was complete, an extraction process was performed thereon by using dichloromethane, and then, column chromatography was performed thereon using hexane and methylenechloride (MC) at a ratio of 4:1 (v:v) to obtain Intermediate 1-1 (yield of 84%). The obtained compound was confirmed by Gas Chromotography-Mass Spectrometry (GC-Mass).

GC-Mass (theoretical value: 405.47 g/mol, measured value: 404 g/mol)

### Synthesis of Intermediate 1-2

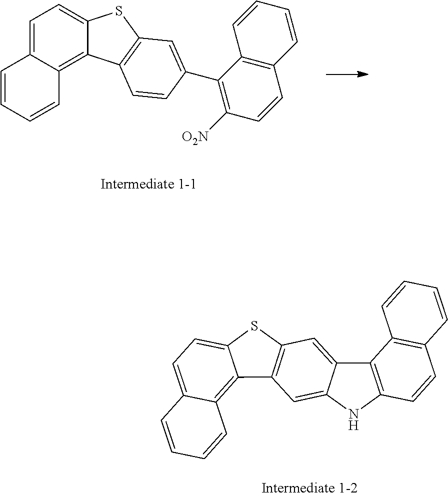 [SMILES: O=[N+]([O-])c1ccc2ccccc2c1-c1ccc2c(c1)sc1ccc3ccccc3c12 ; c1ccc2c(c1)ccc1[nH]c3cc4c(cc3c12)sc1ccc2ccccc2c14]

5 g of Intermediate 1-1 was dissolved in 15 g of triethylphosphite and then reflux-stirred for 12 hours under nitrogen. After the reaction was complete, unreacted triethylphosphite was removed therefrom by vacuum distillation, and column chromatography was performed thereon using hexane and methylenechloride (MC) at a ratio of 4:1 (v/v) to obtain 1.3 g of Intermediate 1-2 (yield of 21.5%). The obtained compound was confirmed by GC-Mass.

GC-Mass (theoretical value: 373.47 g/mol, measured value: 372 g/mol)

### Synthesis of Compound 1

10 g (0.0271 mol) of Intermediate 1-2 (15H-benzo[g]naphtho[1′,2′:4,5]thieno[3,2-b]carbazole), 5.08 g (1.2 eq) of bromobenzene, 0.27 g (0.03 eq, 0.007 mmol) of Pd₂(dba)₃, 3.74 g (1.1 eq, 0.0517 mol) of Na(t-bu)O, and 0.84 g (0.06 eq, 0.002 mmol) of P(t-Bu)₃ were added to a flask and then dissolved in 120 ml of toluene. The resultant mixture was heat-stirred for 12 hours and then cooled to room temperature. An extraction process was performed thereon by using methylenechloride (MC), and the result was washed with distilled water. After dehydration using magnesium sulfate (MgSO₄) and distillation under reduced pressure, a residue was separated therefrom by column chromatography to obtain Compound 1.

The obtained compound was confirmed by Elemental Analysis and High-Resolution Mass Spectrometry (HRMS).

Elemental Analysis for C₃₂H₁₉NS: calcd: C, 85.49; H, 4.26; N, 3.12; S, 7.13.

HRMS for C₃₂H₁₉NS [M]+: calcd: 449.12. found: 448.

### Synthesis Example 2

### Synthesis of Compound 2

### Synthesis of Intermediate 2-1

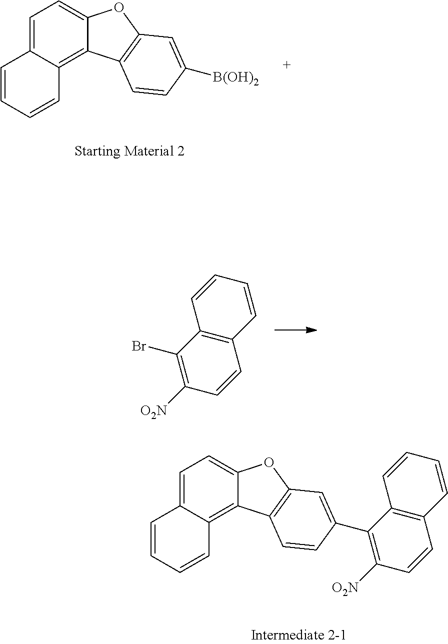 [SMILES: OB(O)c1ccc2c(c1)oc1ccc3ccccc3c12 ; O=[N+]([O-])c1ccc2ccccc2c1Br ; O=[N+]([O-])c1ccc2ccccc2c1-c1ccc2c(c1)oc1ccc3ccccc3c12]

5 g (1 eq) of Starting Material 2, 5.08 g (1.2 eq) of 1-bromo-2-nitronaphthalene, and 0.9 g (0.03 eq) of Pd(PPh₃)₄ were put in a flask and dissolved in 120 ml of toluene while being packed with nitrogen. Next, 40 ml of an aqueous solution in which 10 g of sodium carbonate was dissolved was added to the flask, and then, the result was reflux-stirred for 12 hours. After the reaction was complete, an extraction process was performed thereon by using dichloromethane, and then, column chromatography was performed thereon using hexane and methylenechloride (MC) at a ratio of 4:1 (v:v) to obtain Intermediate 2-1 (yield of 84%). The obtained compound was confirmed by GC-Mass.

GC-Mass (theoretical value: 389.41 g/mol, measured value: 388 g/mol)

### Synthesis of Intermediate 2-2

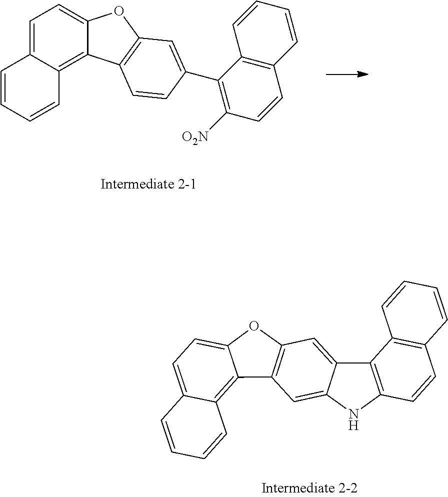 [SMILES: O=[N+]([O-])c1ccc2ccccc2c1-c1ccc2c(c1)oc1ccc3ccccc3c12 ; c1ccc2c(c1)ccc1[nH]c3cc4c(cc3c12)oc1ccc2ccccc2c14]

5 g of Intermediate 2-1 was dissolved in 15 g of triethylphosphite and then reflux-stirred for 12 hours under nitrogen. After the reaction was complete, unreacted triethylphosphite was removed therefrom by vacuum distillation, and column chromatography was performed thereon using hexane and MC at a ratio of 4:1 (v/v) to obtain 1.3 g of Intermediate 2-2 (yield of 21.5%). The obtained compound was confirmed by GC-Mass.

GC-Mass (theoretical value: 357.41 g/mol, measured value: 356 g/mol)

### Synthesis of Compound 2

10 g (0.028 mol) of Intermediate 2-2 (15H-benzo[g]naphtho[1′,2′:4,5]furo[3,2-b]carbazole), 5.01 g (1.2 eq) of bromobenzene, 0.27 g (0.03 eq, 0.007 mmol) of Pd₂(dba)₃, 3.74 g (1.1 eq, 0.0517 mol) of Na(t-bu)O, and 0.84 g (0.06 eq, 0.0015 mmol) of P(t-Bu)₃ were added to a flask and then dissolved in 120 ml of toluene. The resultant mixture was heat-stirred for 12 hours and then cooled to room temperature. An extraction process was performed thereon by using MC, and the result was washed with distilled water. After dehydration by using magnesium sulfate (MgSO₄) and distillation under reduced pressure, a residue was separated therefrom by column chromatography to obtain Compound 2. The obtained compound was confirmed by Elemental Analysis and HRMS.

Elemental Analysis for C₃₂H₁₉NO: calcd: C, 88.66; H, 4.42; N, 3.23; 0, 3.69.

HRMS for C₃₂H₁₉NO [M]+: calcd 433.15. found 432.

### Synthesis Example 3

### Synthesis of Compound 6

### Synthesis of Compound 6

10 g (0.028 mol) of Intermediate 2-2 (15H-benzo[g]naphtho[1′,2′:4,5]furo[3,2-b]carbazole), 9.17 g (1.2 eq) of 2-bromo-9,9-dimethyl-9H-fluorene, 0.27 g (0.03 eq, 0.007 mmol) of Pd₂(dba)₃, 3.74 g (1.1 eq, 0.0517 mol) of Na(t-bu)O, and 0.84 g (0.06 eq, 0.0015 mmol) of P(t-Bu)₃ were added to a flask and then dissolved in 120 ml of toluene. The resultant mixture was heat-stirred for 12 hours and then cooled to room temperature. An extraction process was performed thereon by using methylenechloride (MC), and the result was washed with distilled water. After dehydration by using magnesium sulfate (MgSO₄) and distillation under reduced pressure, a residue was separated therefrom by column chromatography to obtain Compound 6. The obtained compound was confirmed by Elemental Analysis and HRMS.

Elemental Analysis for C₄₁H₂₇NO: calcd: C, 89.59; H, 4.95; N, 2.55; 0, 2.91.

HRMS for C₄₁H₂₇NO [M]+: calcd 549.21. found 548.

### Synthesis Example 4

### Synthesis of Compound 7

### Synthesis of Intermediate 3-1

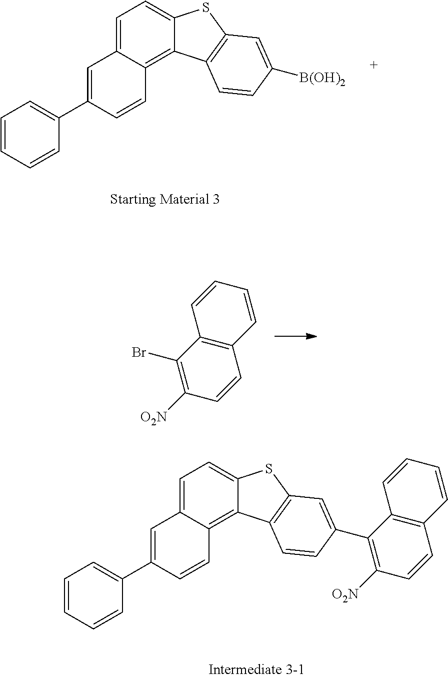 [SMILES: OB(O)c1ccc2c(c1)sc1ccc3cc(-c4ccccc4)ccc3c12 ; O=[N+]([O-])c1ccc2ccccc2c1Br ; O=[N+]([O-])c1ccc2ccccc2c1-c1ccc2c(c1)sc1ccc3cc(-c4ccccc4)ccc3c12]

5 g (1 eq) of Starting Material 3, 5.08 g (1.2 eq) of 1-bromo-2-nitronaphthalene, and 0.9 g (0.03 eq) of Pd(PPh₃)₄ were put in a flask and dissolved in 120 ml of toluene while being packed with nitrogen. Next, 40 ml of an aqueous solution in which 10 g of sodium carbonate was dissolved was added to the flask, and then, the result was reflux-stirred for 12 hours. After the reaction was complete, an extraction process was performed thereon by using dichloromethane, and then, column chromatography was performed thereon using hexane and methylenechloride (MC) at a ratio of 4:1 (v:v) to obtain Intermediate 3-1 (yield of 84%). The obtained compound was confirmed by GC-Mass.

GC-Mass (theoretical value: 481.57 g/mol, measured value: 480 g/mol)

### Synthesis of Intermediate 3-2

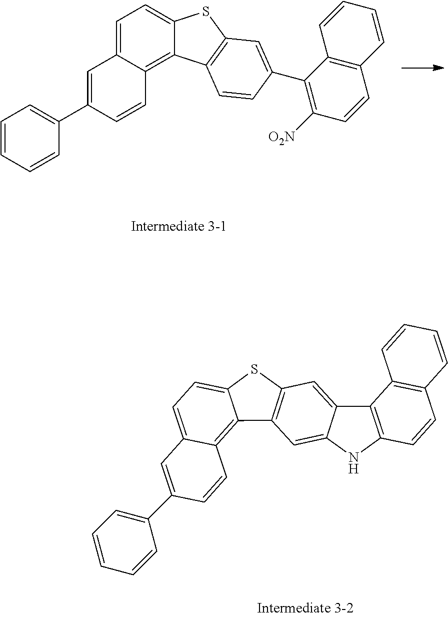 [SMILES: O=[N+]([O-])c1ccc2ccccc2c1-c1ccc2c(c1)sc1ccc3cc(-c4ccccc4)ccc3c12 ; c1ccc(-c2ccc3c(ccc4sc5cc6c(cc5c43)[nH]c3ccc4ccccc4c36)c2)cc1]

5 g of Intermediate 3-1 was dissolved in 15 g of triethylphosphite and then reflux-stirred for 12 hours under nitrogen. After the reaction was complete, unreacted triethylphosphite was removed therefrom by vacuum distillation, and column chromatography was performed thereon using hexane and MC at a ratio of 4:1 (v/v) to obtain 1.3 g of Intermediate 3-2 (yield of 21.5%). The obtained compound was confirmed by GC-Mass.

GC-Mass (theoretical value: 449.57 g/mol, measured value: 448 g/mol)

### Synthesis of Compound 7

10 g (0.0222 mol) of Intermediate 3-2 (11-phenyl-15H-benzo[g]naphtho[1′,2′:4,5]thieno[3,2-b]carbazole), 4.16 g (1.2 eq) of bromobenzene, 0.21 g (0.03 eq, 0.0006 mmol) of Pd₂(dba)₃, 1.67 g (1.1 eq, 0.0242 mol) of Na(t-bu)O, and 0.81 g (0.06 eq, 0.0013 mmol) of P(t-Bu)₃ were added to a flask and then dissolved in 120 ml of toluene. The resultant mixture was heat-stirred for 12 hours and then cooled to room temperature. An extraction process was performed thereon by using MC, and the result was washed with distilled water. After dehydration by using magnesium sulfate (MgSO₄) and distillation under reduced pressure, a residue was separated therefrom by column chromatography to obtain Compound 7. The obtained compound was confirmed by Elemental Analysis and HRMS.

Elemental Analysis for C₃₈H₂₃NS: calcd: C, 86.83; H, 4.41; N, 2.66; S, 6.10.

HRMS for C₃₈H₂₃NS [M]+: calcd 525.67. found 524.

### Synthesis Example 5

### Synthesis of Compound 11

### Synthesis of Compound 11

10 g (0.0271 mol) of Intermediate 1-2 (15H-benzo[g]naphtho[1′,2′:4,5]thieno[3,2-b]carbazole), 4.25 g (1.2 eq) of bromopyridine, 0.27 g (0.03 eq, 0.007 mmol) of Pd₂(dba)₃, 3.74 g (1.1 eq, 0.0517 mol) of Na(t-bu)O, and 0.84 g (0.06 eq, 0.002 mmol) of P(t-Bu)₃ were added to a flask and then dissolved in 120 ml of toluene. The resultant mixture was heat-stirred for 12 hours and then cooled to room temperature. An extraction process was performed thereon by using methylenechloride (MC), and the result was washed with distilled water. After dehydration by using magnesium sulfate (MgSO₄) and distillation under reduced pressure, a residue was separated therefrom by column chromatography to obtain Compound 11. The obtained compound was confirmed by Elemental Analysis and HRMS.

Elemental Analysis for C₃₁H₁₈N₂S: calcd: C, 82.64; H, 4.03; N, 6.22; S, 7.12.

HRMS for C₃₁H₁₈N₂S [M]+: calcd 450.56. found 449.

### Synthesis Example 6

### Synthesis of Compound 12

### Synthesis of Compound 12

10 g (0.028 mol) of Intermediate 2-2 (15H-benzo[g]naphtho[1′,2′:4,5]furo[3,2-b]carbazole), 4.12 g (1.2 eq) of bromopyridine, 0.27 g (0.03 eq, 0.007 mmol) of Pd₂(dba)₃, 3.74 g (1.1 eq, 0.0517 mol) of Na(t-bu)O, and 0.84 g (0.06 eq, 0.0015 mmol) of P(t-Bu)₃ were added to a flask and then dissolved in 120 ml of toluene. The resultant mixture was heat-stirred for 12 hours and then cooled to room temperature. An extraction process was performed thereon by using methylenechloride (MC), and the result was washed with distilled water. After dehydration by using magnesium sulfate (MgSO₄) and distillation under reduced pressure, a residue was separated therefrom by column chromatography to obtain Compound 12. The obtained compound was confirmed by Elemental Analysis and HRMS.

Elemental Analysis for C₃₁H₁₈N₂O: calcd: C, 85.69; H, 4.18; N, 6.45; 0, 3.68.

HRMS for C₃₁H₁₈N₂O [M]+: calcd 434.50. found 433.

### Synthesis Example 7

### Synthesis of Compound 13

### Synthesis of Compound 13

10 g (0.0271 mol) of Intermediate 1-2 (15H-benzo[g]naphtho[1′,2′:4,5]thieno[3,2-b]carbazole), 10.08 g (1.2 eq) of 2-bromo-4,6-diphenylpyrimidine, 0.27 g (0.03 eq, 0.007 mmol) of Pd₂(dba)₃, 3.74 g (1.1 eq, 0.0517 mol) of Na(t-bu)O, and 0.84 g (0.06 eq, 0.002 mmol) of P(t-Bu)₃ were added to a flask and then dissolved in 120 ml of toluene. The resultant mixture was heat-stirred for 12 hours and then cooled to room temperature. An extraction process was performed thereon by using methylenechloride (MC), and the result was washed with distilled water. After dehydration by using magnesium sulfate (MgSO₄) and distillation under reduced pressure, a residue was separated therefrom by column chromatography to obtain Compound 13. The obtained compound was confirmed by Elemental Analysis and HRMS.

Elemental Analysis for C₄₂H₂₅N₃S: calcd: C, 83.56; H, 4.17; N, 6.96; S, 5.31.

HRMS for C₄₂H₂₅N₃S [M]+: calcd 603.74. found 602.

### Synthesis Example 8

### Synthesis of Compound 16

### Synthesis of Compound 16

10 g (0.028 mol) of Intermediate 2-2 (15H-benzo[g]naphtho[1′,2′:4,5]furo[3,2-b]carbazole), 10.2 g (1.2 eq) of 2-bromo-4,6-diphenyl-1,3,5-triazine, 0.27 g (0.03 eq, 0.007 mmol) of Pd₂(dba)₃, 3.74 g (1.1 eq, 0.0517 mol) of Na(t-bu)O, and 0.84 g (0.06 eq, 0.0015 mmol) of P(t-Bu)₃ were added to a flask and then dissolved in 120 ml of toluene. The resultant mixture was heat-stirred for 12 hours and then cooled to room temperature. An extraction process was performed thereon by using methylenechloride (MC), and the result was washed with distilled water. After dehydration by using magnesium sulfate (MgSO₄) and distillation under reduced pressure, a residue was separated therefrom by column chromatography to obtain Compound 16. The obtained compound was confirmed by Elemental Analysis and HRMS.

Elemental Analysis for C₄₁H₂₄N₄O: calcd: C, 83.65; H, 4.11; N, 9.52; O, 2.72.

HRMS for C₄₁H₂₄N₄O [M]+: calcd 588.67. found 587.

### Example 1

A 15 Ω/cm² (1200 Å) ITO glass substrate (available from Corning Co., Ltd) was cut to a size of 50 mm×50 mm×0.7 mm, sonicated in isopropyl alcohol and pure water for 5 minutes in each solvent, cleaned with ultraviolet rays for 30 minutes, and then ozone, and was mounted on a vacuum deposition apparatus.

Compound 2-TNATA was vacuum-deposited on an ITO anode of the glass substrate to form a hole injection layer (HIL) having a thickness of about 600 Å. Then, Compound NPB was vacuum-deposited on the HIL to form a hole transport layer (HTL) having a thickness of about 300 Å, thereby forming a hole transport region.

Compound 1 (as a host), and PD1 (as a dopant), were co-deposited on the hole transport region at a weight ratio of about 85:15, thereby forming an emission layer having a thickness of about 300 Å.

Alq₃ was vacuum-deposited on the emission layer to form an electron transport layer (ETL) having a thickness of about 300 Å. Then, LiF was deposited on the ETL to form an electron injection layer (EIL) having a thickness of about 10 Å, thereby forming an electron transport region.

Aluminum (Al) was vacuum-deposited on the electron transport region to form a cathode having a thickness of about 1200 Å, thereby completing the manufacture of an organic light-emitting device.

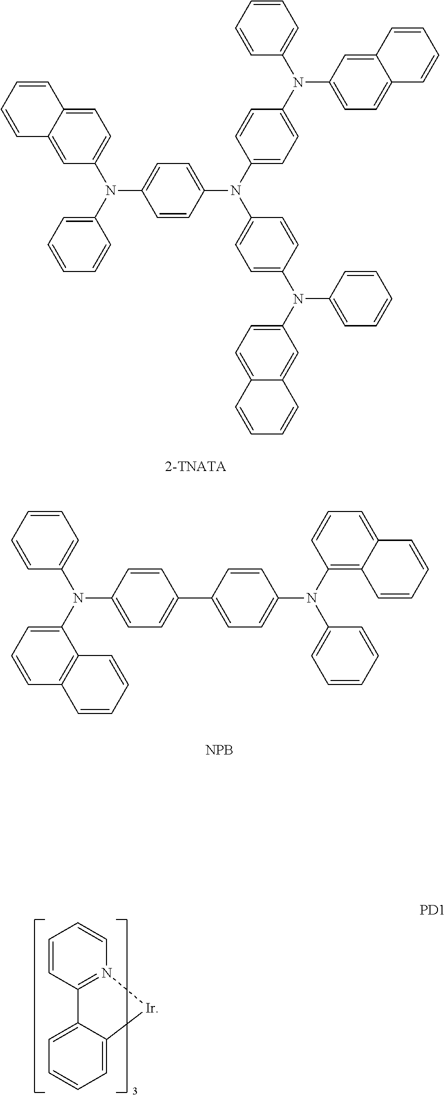

### Examples 2 to 4 and Comparative Examples 1 to 3

Organic light-emitting devices of Examples 2 to 4 and Comparative Examples 1 to 3 were each manufactured in the same (or substantially the same) manner as in Example 1, except that Compounds 2, 6, 7 and Compounds A, B, and C were respectively used as a host, instead of Compound 1, to form an emission layer.

### Evaluation Example 1

The efficiency and lifespan (T₉₅) of the organic light-emitting devices manufactured according to Examples 1 to 4 and Comparative Examples 1 to 3 were measured by using Keithley SMU 236 and a luminance meter PR650 (Photo Research, Inc.), and results thereof are shown in Table 1. Here, the lifespan (T₉₅) is a period of time that lapses until the luminance of an organic light-emitting device is reduced to 95% of the initial luminance.

TABLE 1

| | | | | Effi- | Lifespan |
|---|---|---|---|---|---|
| | Emission Layer | | | ciency | (T₉₅) |
| | Host | Dopant | Weight Ratio | (cd/A) | (1000 nit) |
| Example 1 | Compound 1 | PD1 | 85:15 | 44.1 | 921 |
| Example 2 | Compound 2 | PD1 | 85:15 | 45 | 943 |
| Example 3 | Compound 6 | PD1 | 85:15 | 46.8 | 897 |
| Example 4 | Compound 7 | PD1 | 85:15 | 46.1 | 956 |
| Comparative | Compound A | PD1 | 85:15 | 40.1 | 749 |
| Example 1 | | | | | |
| Comparative | Compound B | PD1 | 85:15 | 42.4 | 801 |
| Example 2 | | | | | |
| Comparative | Compound C | PD1 | 85:15 | 42.7 | 884 |
| Example 3 | | | | | |

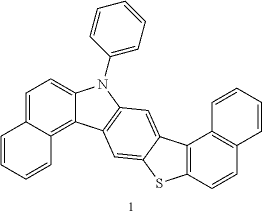 [SMILES: c1ccc(-n2c3cc4c(cc3c3c5ccccc5ccc32)sc2ccc3ccccc3c24)cc1]

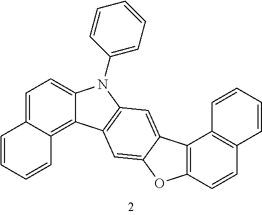 [SMILES: c1ccc(-n2c3cc4c(cc3c3c5ccccc5ccc32)oc2ccc3ccccc3c24)cc1]

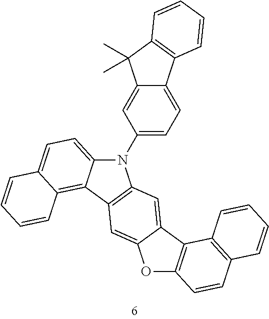 [SMILES: CC1(C)c2ccccc2-c2ccc(-n3c4cc5c(cc4c4c6ccccc6ccc43)oc3ccc4ccccc4c35)cc21]

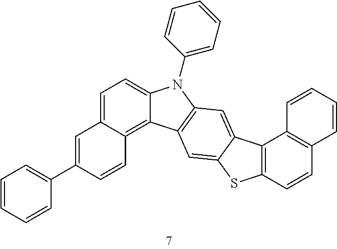 [SMILES: c1ccc(-c2ccc3c(ccc4c3c3cc5sc6ccc7ccccc7c6c5cc3n4-c3ccccc3)c2)cc1]

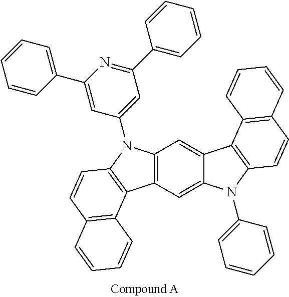 [SMILES: c1ccc(-c2cc(-n3c4cc5c6c7ccccc7ccc6n(-c6ccccc6)c5cc4c4c5ccccc5ccc43)cc(-c3ccccc3)n2)cc1]

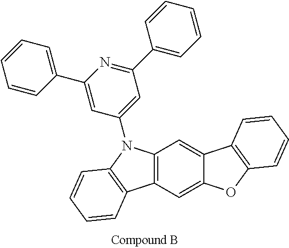 [SMILES: c1ccc(-c2cc(-n3c4ccccc4c4cc5oc6ccccc6c5cc43)cc(-c3ccccc3)n2)cc1]

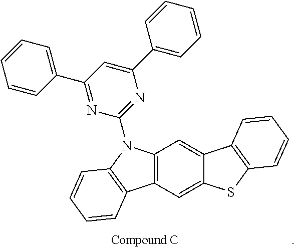 [SMILES: c1ccc(-c2cc(-c3ccccc3)nc(-n3c4ccccc4c4cc5sc6ccccc6c5cc43)n2)cc1]

From the results shown in Table 1, it can be seen that the organic light-emitting devices of Examples 1 to 4 had a higher efficiency and a longer lifespan than the organic light-emitting devices of Comparative Examples 1 and 3.

### Example 5

A 15 Ω/cm² (1200 Å) ITO glass substrate (available from Corning Co., Ltd) was cut to a size of 50 mm×50 mm×0.7 mm, sonicated in isopropyl alcohol and pure water for 5 minutes in each solvent, cleaned with ultraviolet rays for 30 minutes, and then ozone, and was mounted on a vacuum deposition apparatus.

Compound 2-TNATA was vacuum-deposited on an ITO anode of the glass substrate to form a hole injection layer (HIL) having a thickness of about 600 Å. Then, Compound NPB was vacuum-deposited on the HIL to form a hole transport layer (HTL) having a thickness of about 300 Å, thereby forming a hole transport region.

Compound 11 (as a host), and PD16 (as a dopant), were co-deposited on the hole transport region at a weight ratio of about 95:5, thereby forming an emission layer having a thickness of about 200 Å.

Alq₃ was vacuum-deposited on the emission layer to form an electron transport layer (ETL) having a thickness of about 300 Å. Then, LiF was deposited on the ETL to form an electron injection layer (EIL) having a thickness of about 10 Å, thereby forming an electron transport region.

Aluminum (Al) was vacuum-deposited on the electron transport region to form a cathode having a thickness of about 1200 Å, thereby completing the manufacture of an organic light-emitting device.

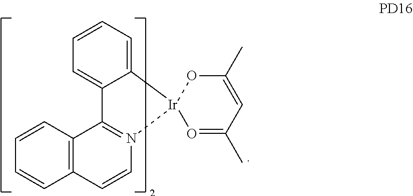

### Examples 6 to 8 and Comparative Examples 4 to 7

Organic light-emitting devices of Examples 6 to 8 and Comparative Examples 4 to 7 were each manufactured in the same (or substantially the same) manner as in Example 5, except that Compounds 12, 13, 16, bis(2-methyl-8-quinolinolato-N1,O8)-(1,1′-Biphenyl-4-olato)aluminum (BAlq), and Compounds A, B, and C were respectively used as a host, instead of Compound 11, to form an emission layer.

### Evaluation Example 2

The efficiency and lifespan (T₉₅) of the organic light-emitting devices manufactured according to Examples 5 to 8 and Comparative Examples 4 to 7 were measured by using Keithley SMU 236 and a luminance meter PR650 (Photo Research, Inc.), and results thereof are shown in Table 2. Here, the lifespan (T₉₅) is a period of time that lapses until the luminance of an organic light-emitting device is reduced to 95% of the initial luminance.

TABLE 2

| | | | | Effi- | Lifespan |
|---|---|---|---|---|---|
| | Emission Layer | | | ciency | (T₉₅) |
| | Host | Dopant | Weight Ratio | (cd/A) | (700 nit) |
| Example 5 | Compound 11 | PD16 | 95:5 | 25.7 | 1775 |
| Example 6 | Compound 12 | PD16 | 95:5 | 29.8 | 1711 |
| Example 7 | Compound 13 | PD16 | 95:5 | 25.9 | 1734 |
| Example 8 | Compound 16 | PD16 | 95:5 | 26.9 | 1788 |
| Comparative | BAlq | PD16 | 95:5 | 18.2 | 1345 |
| Example 4 | | | | | |
| Comparative | Compound A | PD16 | 95:5 | 20.7 | 1621 |
| Example 5 | | | | | |
| Comparative | Compound B | PD16 | 95:5 | 21.8 | 1643 |
| Example 6 | | | | | |
| Comparative | Compound C | PD16 | 95:5 | 22.4 | 1699 |
| Example 7 | | | | | |

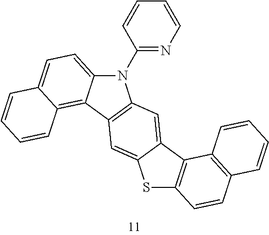 [SMILES: c1ccc(-n2c3cc4c(cc3c3c5ccccc5ccc32)sc2ccc3ccccc3c24)nc1]

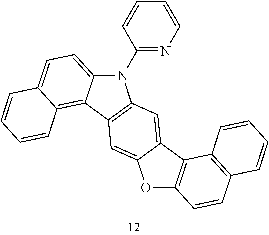 [SMILES: c1ccc(-n2c3cc4c(cc3c3c5ccccc5ccc32)oc2ccc3ccccc3c24)nc1]

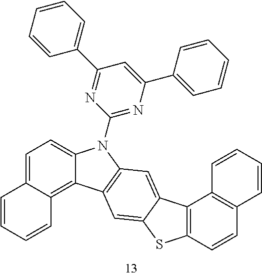 [SMILES: c1ccc(-c2cc(-c3ccccc3)nc(-n3c4cc5c(cc4c4c6ccccc6ccc43)sc3ccc4ccccc4c35)n2)cc1]

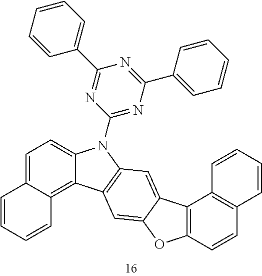 [SMILES: c1ccc(-c2nc(-c3ccccc3)nc(-n3c4cc5c(cc4c4c6ccccc6ccc43)oc3ccc4ccccc4c35)n2)cc1]

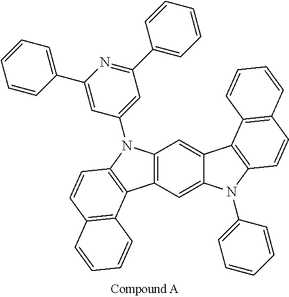 [SMILES: c1ccc(-c2cc(-n3c4cc5c6c7ccccc7ccc6n(-c6ccccc6)c5cc4c4c5ccccc5ccc43)cc(-c3ccccc3)n2)cc1]

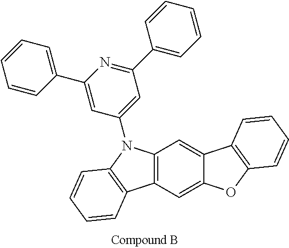 [SMILES: c1ccc(-c2cc(-n3c4ccccc4c4cc5oc6ccccc6c5cc43)cc(-c3ccccc3)n2)cc1]

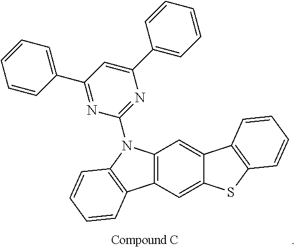 [SMILES: c1ccc(-c2cc(-c3ccccc3)nc(-n3c4ccccc4c4cc5sc6ccccc6c5cc43)n2)cc1]

From the results shown in Table 2, it can be seen that the organic light-emitting devices of Examples 5 to 8 had a higher efficiency and a longer lifespan than the organic light-emitting devices of Comparative Examples 4 and 7.

According to one or more embodiments of the present disclosure, an organic light-emitting device including the above-described condensed cyclic compound may have a low driving voltage, a high efficiency, a high luminance, and a long lifespan.

It will be understood that the terms, such as “comprises,” “comprising,” “includes”, and/or “including”, when used in this specification, specify the presence of stated features, integers, steps, operations, elements, and/or components, but do not preclude the presence or addition of one or more other features, integers, steps, operations, elements, components, and/or groups thereof.

In addition, as used herein, the terms “use,” “using,” and “used” may be considered synonymous with the terms “utilize,” “utilizing,” and “utilized,” respectively. As used herein, the term “substantially,” “about,” and similar terms are used as terms of approximation and not as terms of degree, and are intended to account for the inherent deviations in measured or calculated values that would be recognized by those of ordinary skill in the art.

Also, any numerical range recited herein is intended to include all subranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. §112(a) and 35 U.S.C. §132(a).

It should be understood that example embodiments described herein should be considered in a descriptive sense only and not for purposes of limitation. Descriptions of features or aspects within each example embodiment should typically be considered as available for other similar features or aspects in other example embodiments.

While one or more example embodiments have been described with reference to the drawing, it will be understood by those of ordinary skill in the art that various changes in form and details may be made therein without departing from the spirit and scope of the present disclosure as defined by the following claims and equivalents thereof.

What is claimed is:

1. A condensed cyclic compound represented by Formula 1: 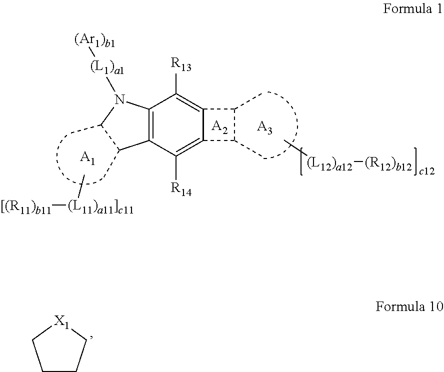
wherein, in Formulae 1 and 10,
A₂ ring and A₃ ring are fused to each other,
A₁ ring and A₃ ring are each independently a naphthalene ring,
A₂ ring is represented by Formula 10, and X₁ in Formula 10 is an oxygen atom (O) or a sulfur atom (s),
L₁, L₁₁, and L₁₂ are each independently selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,
a1, a11, and a12 are each independently selected from 0, 1, 2, and 3, when a1 is 2 or more, two or more L₁(s) are identical to or different from each other, when a11 is 2 or more, two or more L₁₁(s) are identical to or different from each other, and when a12 is 2 or more, two or more L₁₂(s) are identical to or different from each other,
Ar₁ is selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group,
b1 is selected from 1, 2, and 3, and when b1 is 2 or more, two or more Ar₁(s) are identical to or different from each other,
R₁₁ to R₁₄ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), and N(Q₆)(Q₇),
b11 and b12 are each independently selected from 0, 1, 2, and 3, when b11 is 2 or more, two or more R₁₁(s) are identical to or different from each other, and when b12 is 2 or more, two or more R₁₂(s) are identical to or different from each other,
c11 and c12 are each independently selected from 0, 1, 2, 3, 4, 5, and 6, when c11 is 2 or more, two or more *-[(L₁₁)ₐ₁₁-(R₁₁)_{b11}](s) are identical to or different from each other, and when c12 is 2 or more, two or more *-[(L₁₂)ₐ₁₂-(R₁₂)_{b12}](s) are identical to or different from each other, and
at least one substituent of the substituted C₃-C₁₀ cycloalkylene group, substituted C₁-C₁₀ heterocycloalkylene group, substituted C₃-C₁₀ cycloalkenylene group, substituted C₁-C₁₀ heterocycloalkenylene group, substituted C₆-C₆₀ arylene group, substituted C₁-C₆₀ heteroarylene group, substituted divalent non-aromatic condensed polycyclic group, substituted divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₁-C₆₀ heteroaryl group, substituted monovalent non-aromatic condensed polycyclic group, and substituted monovalent non-aromatic condensed heteropolycyclic group is selected from:
deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —B(Q₁₄)(Q₁₅), and —N(Q₁₆)(Q₁₇);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —B(Q₂₄)(Q₂₅), and —N(Q₂₆)(Q₂₇); and
—Si(Q₃₁)(Q₃₂)(Q₃₃), —B(Q₃₄)(Q₃₅), and —N(Q₃₆)(Q₃₇),
wherein Q₁ to Q₇, Q₁₁ to Q₁₇, Q₂₁ to Q₂₇, and Q₃₁ to Q₃₇ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

2. The condensed cyclic compound of claim 1, wherein L₁, L₁₁, and L₁₂ are each independently selected from:
a phenylene group, a pentalenylene group, an indenylene group, a naphthylene group, an azulenylene group, a heptalenylene group, an indacenylene group, an acenaphthylene group, a fluorenylene group, a spiro-fluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a pyrrolylene group, a thiophenylene group, a furanylene group, an imidazolylene group, a pyrazolylene group, a thiazolylene group, an isothiazolylene group, an oxazolylene group, an isoxazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzoimidazolylene group, a benzofuranylene group, a benzothiophenylene group, an isobenzothiazolylene group, a benzoxazolylene group, an isobenzoxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, a thiadiazolylene group, an imidazopyridinylene group, and an imidazopyrimidinylene group; and
a phenylene group, a pentalenylene group, an indenylene group, a naphthylene group, an azulenylene group, a heptalenylene group, an indacenylene group, an acenaphthylene group, a fluorenylene group, a spiro-fluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a pyrrolylene group, a thiophenylene group, a furanylene group, an imidazolylene group, a pyrazolylene group, a thiazolylene group, an isothiazolylene group, an oxazolylene group, an isoxazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzoimidazolylene group, a benzofuranylene group, a benzothiophenylene group, an isobenzothiazolylene group, a benzoxazolylene group, an isobenzoxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, a thiadiazolylene group, an imidazopyridinylene group, and an imidazopyrimidinylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group.

3. The condensed cyclic compound of claim 1, wherein L₁, L₁₁, and L₁₂ are each independently selected from groups represented by Formulae 3-1 to 3-38: 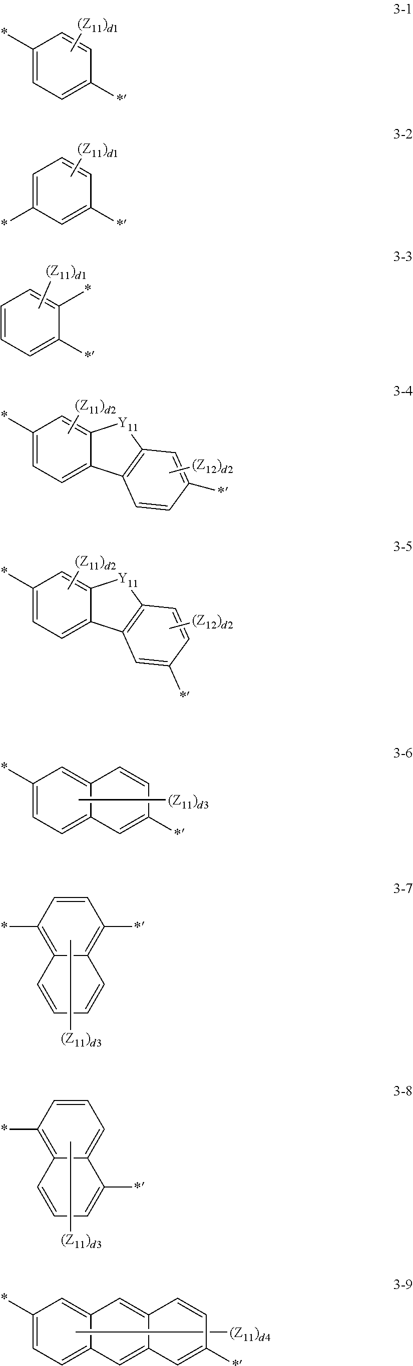 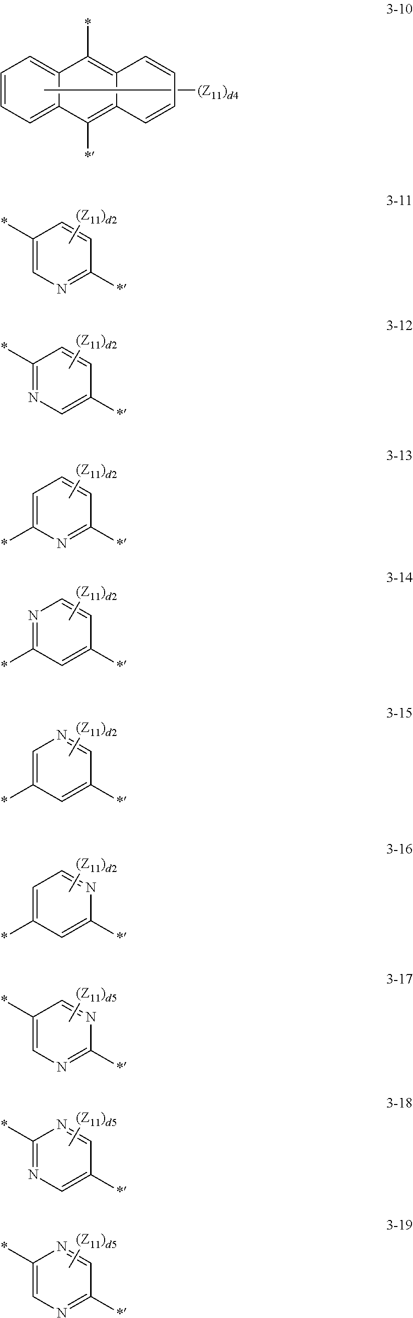 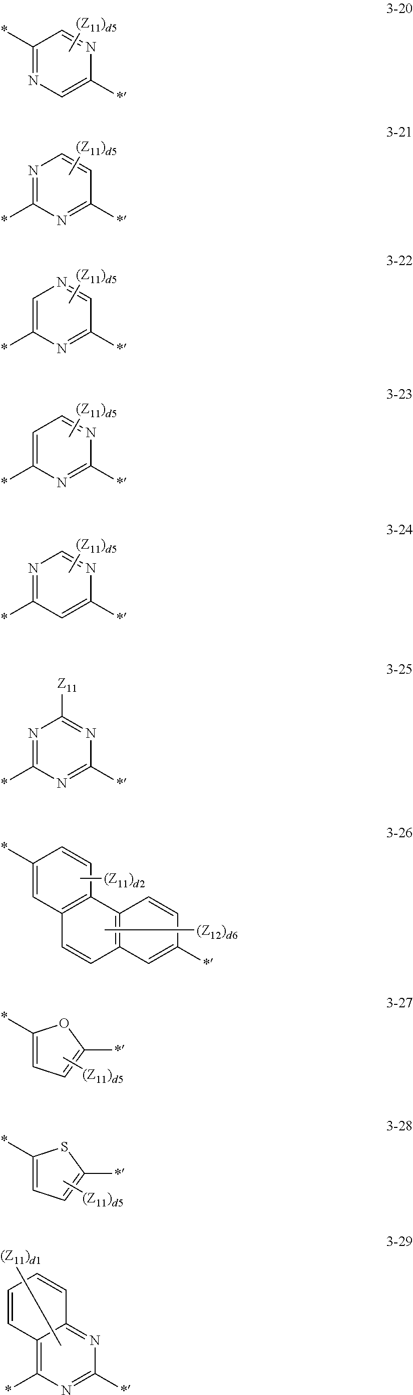 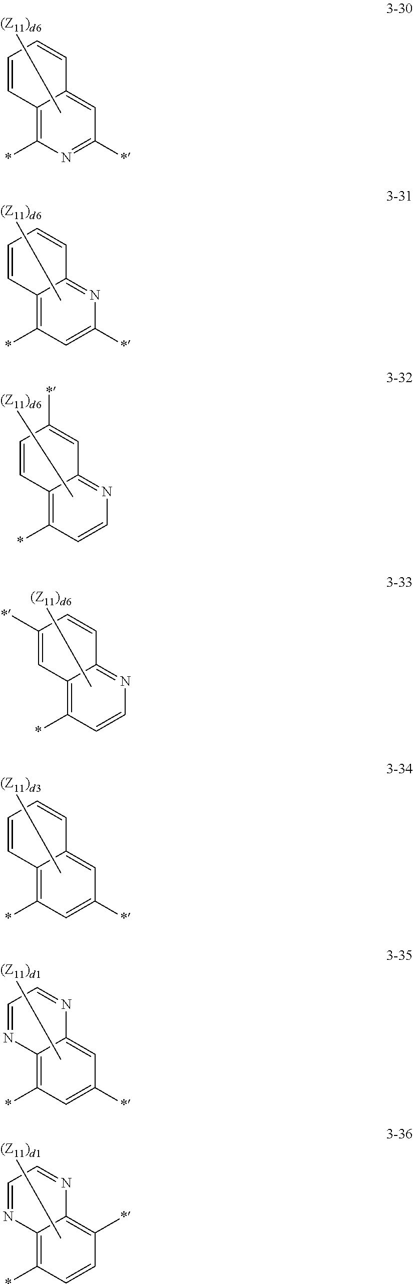 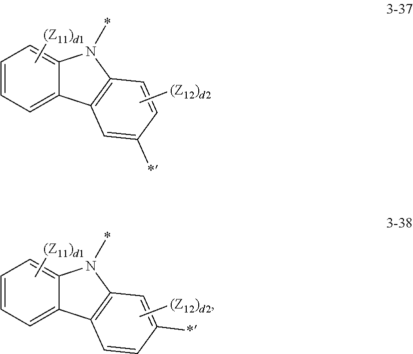
wherein, in Formulae 3-1 to 3-38,
Y₁₁ is selected from O, S, C(Z₁₃)(Z₁₄), N(Z₁₅), and Si(Z₁₆)(Z₁₇),
Z₁₁ to Z₁₇ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,
d1 is an integer selected from 1, 2, 3, and 4, d2 is an integer selected from 1, 2, and 3, d3 is an integer selected from 1, 2, 3, 4, 5, and 6, d4 is an integer selected from 1, 2, 3, 4, 5, 6, 7, and 8, d5 is 1 or 2, d6 is an integer selected from 1, 2, 3, 4, and 5, and each of * and *′ indicates a binding site to a neighboring atom.

4. The condensed cyclic compound of claim 1, wherein L₁, L₁₁, and L₁₂ are each independently selected from groups represented by Formulae 4-1 to 4-25: 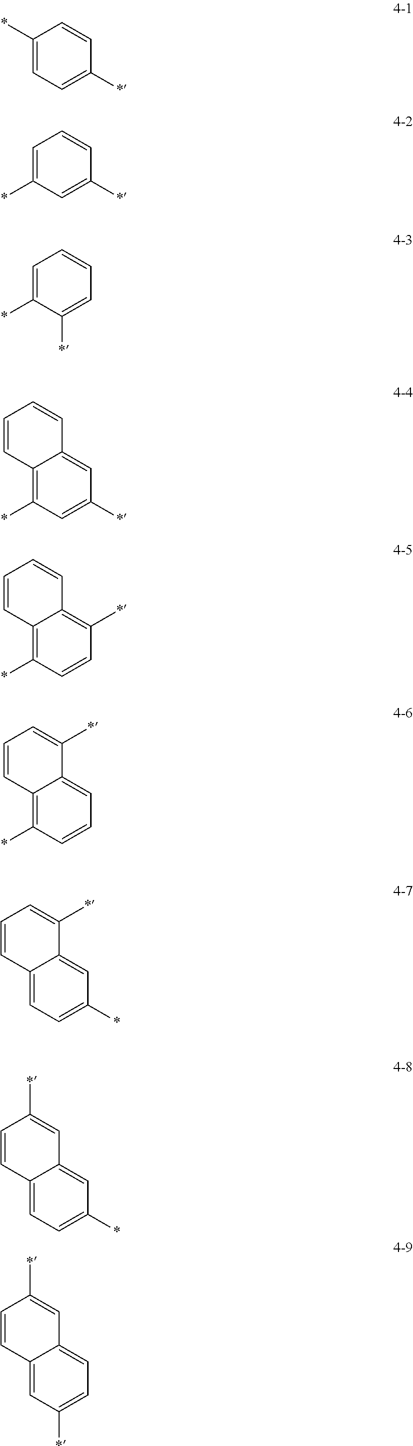 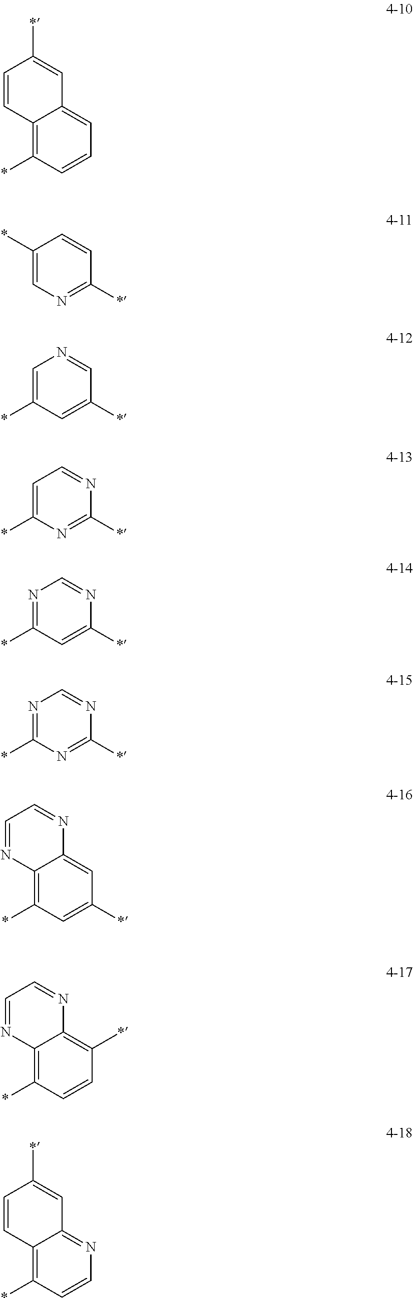 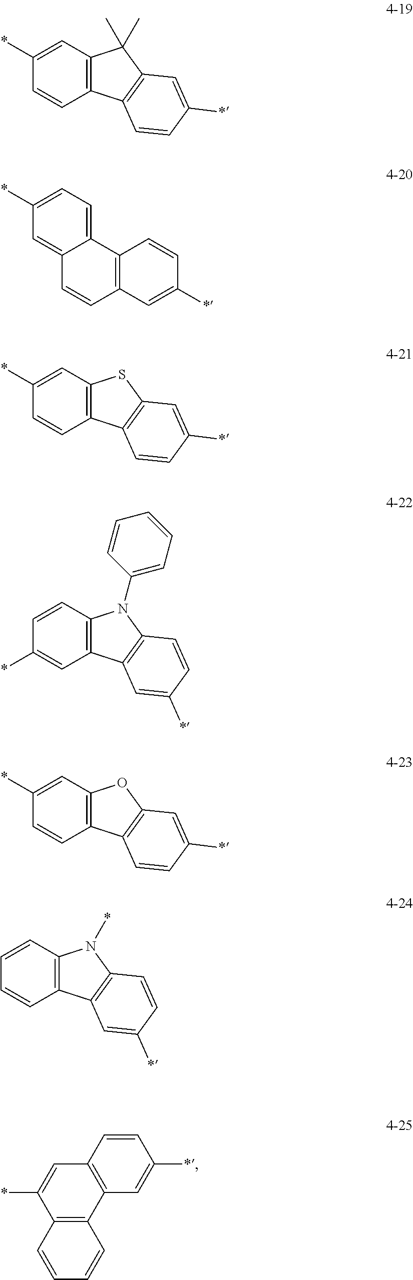
wherein, each of * and *′ in Formulae 4-1 to 4-25 indicates a binding site to a neighboring atom.

5. The condensed cyclic compound of claim 1, wherein a1, a11, and a12 are each independently an integer selected from 0 and 1.

6. The condensed cyclic compound of claim 1, wherein Ar₁ is selected from:
a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a dibenzosilolyl group, a thiadiazolyl group, an imidazopyridinyl group, and an imidazopyrimidinyl group; and
a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a dibenzosilolyl group, a thiadiazolyl group, an imidazopyridinyl group, and an imidazopyrimidinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, and —Si(Q₃₁)(Q₃₂)(Q₃₃),
wherein Q₃₁ to Q₃₃ are each independently selected from a C₁-C₁₀ alkyl group, a C₁-C₁₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a carbazolyl group, a phenanthrolinyl group, a benzoimidazolyl group, a benzofuranyl group, a benzothiophenyl group, an isobenzothiazolyl group, a benzoxazolyl group, an isobenzoxazolyl group, a triazolyl group, an oxadiazolyl group, a triazinyl group, a dibenzofuranyl group, a dibenzothiophenyl group, a benzocarbazolyl group, a dibenzocarbazolyl group, a dibenzosilolyl group, a thiadiazolyl group, an imidazopyridinyl group, and an imidazopyrimidinyl group.

7. The condensed cyclic compound of claim 1, wherein Ar₁ is selected from groups represented by Formulae 5-1 to 5-42: 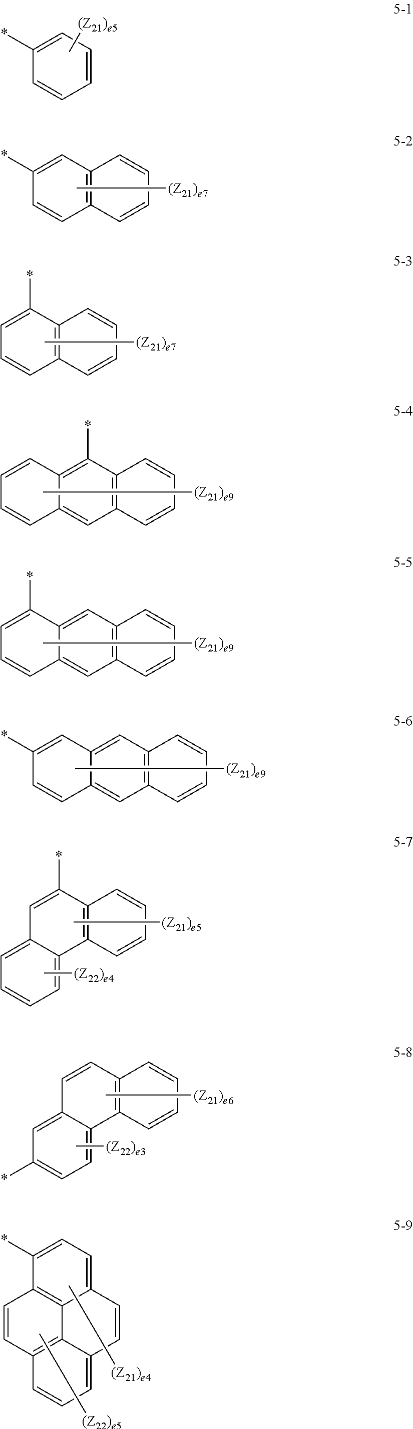 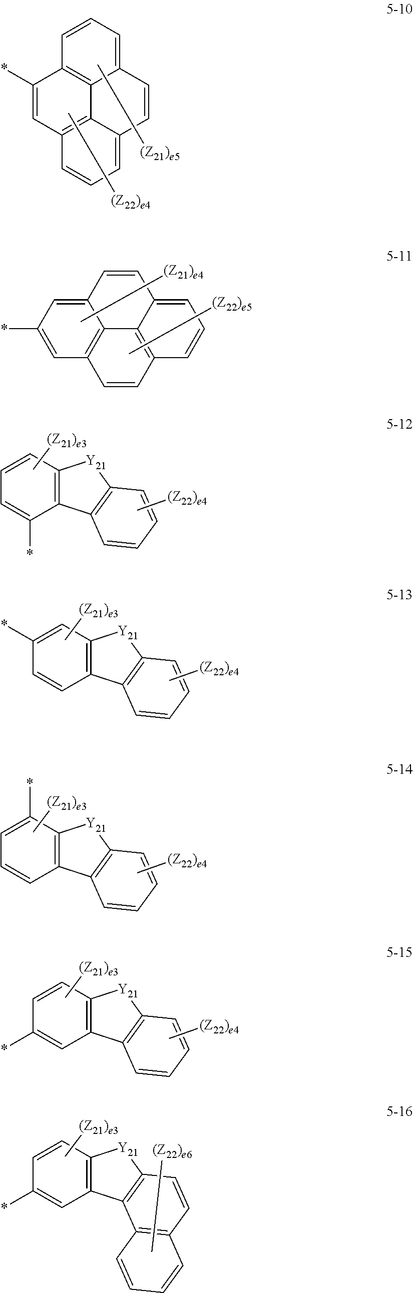 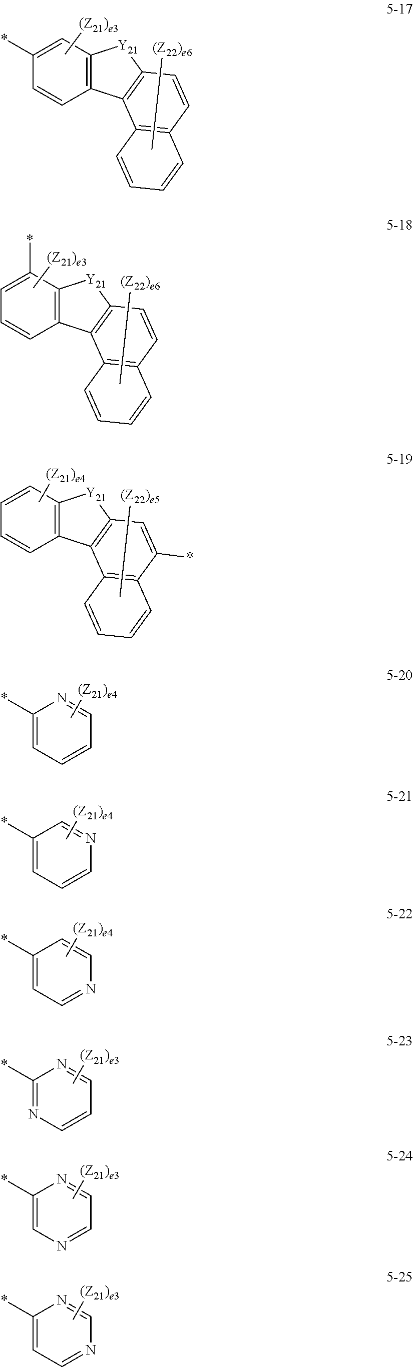 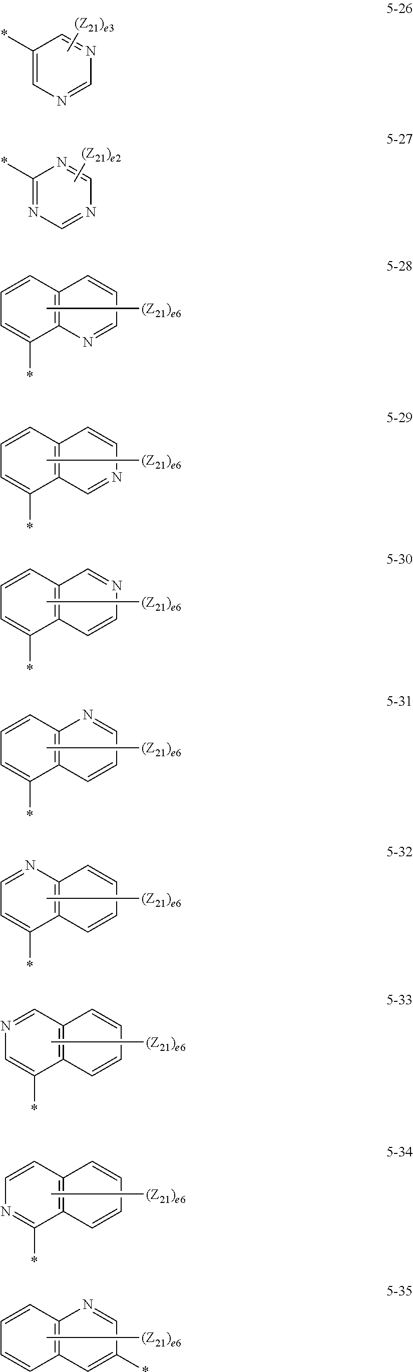 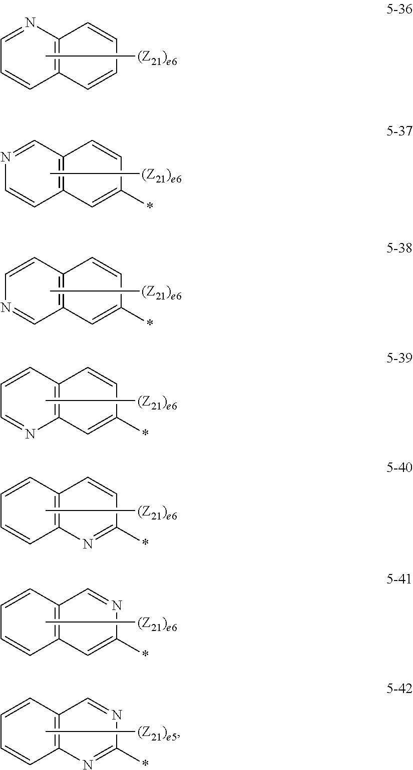
wherein, in Formulae 5-1 to 5-42,
Y₂₁ is selected from O, S, C(Z₂₃)(Z₂₄), N(Z₂₅), and Si(Z₂₆)(Z₂₇),
Z₂₁ to Z₂₇ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,
e2 is 1 or 2, e3 is an integer selected from 1, 2, and 3, e4 is an integer selected from 1, 2, 3, and 4, e5 is an integer selected from 1, 2, 3, 4, and 5, e6 is an integer selected from 1, 2, 3, 4, 5, and 6, e7 is an integer selected from 1, 2, 3, 4, 5, 6, and 7, e9 is an integer selected from 1, 2, 3, 4, 5, 6, 7, 8, and 9, and * indicates a binding site to a neighboring atom.

8. The condensed cyclic compound of claim 1, wherein Ar₁ is selected from groups represented by Formulae 6-1 to 6-43: 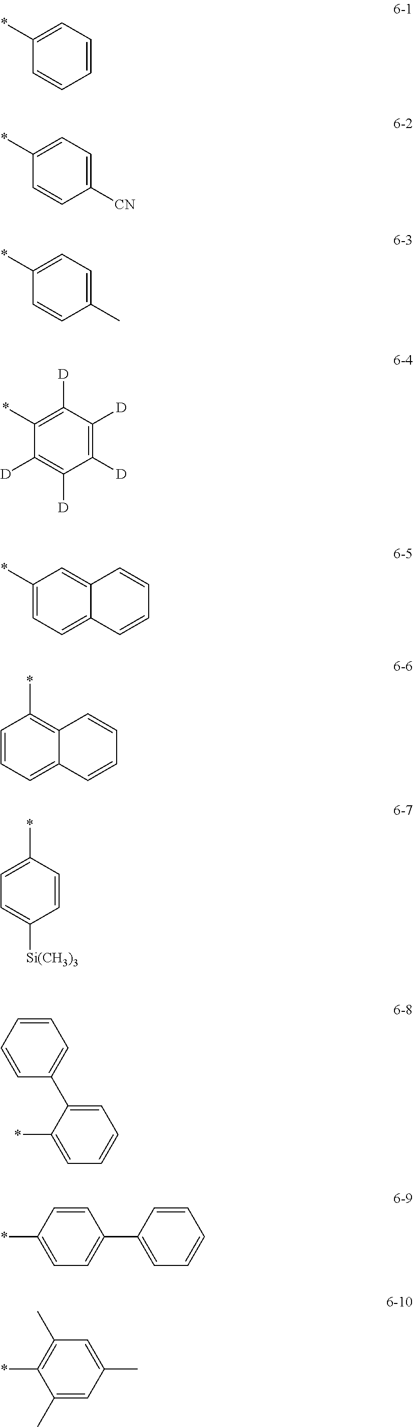 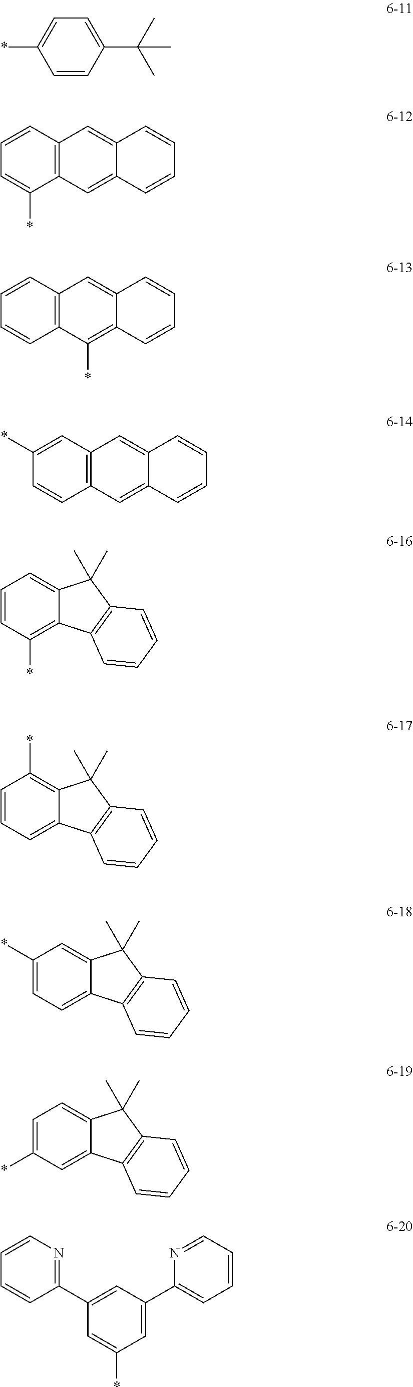 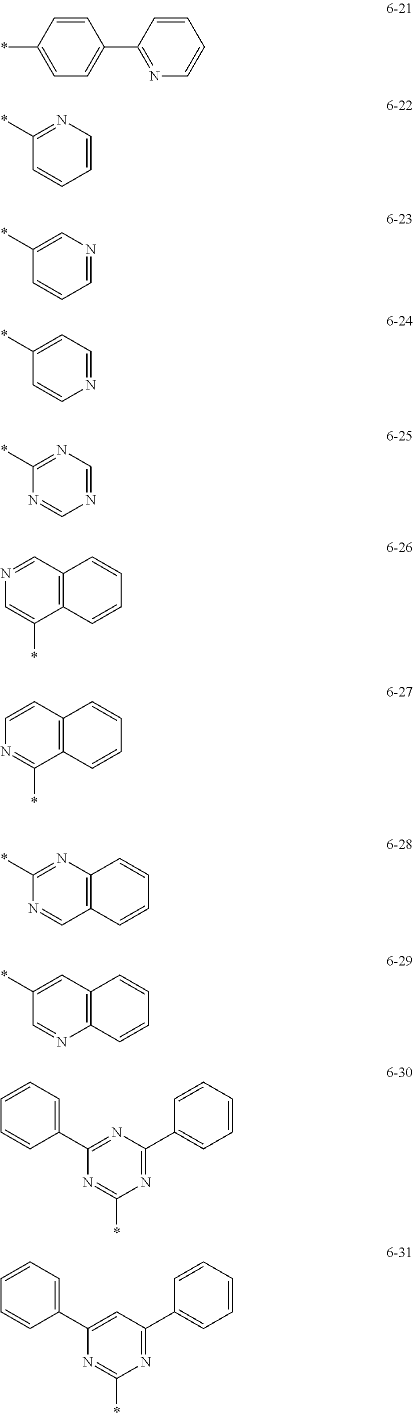 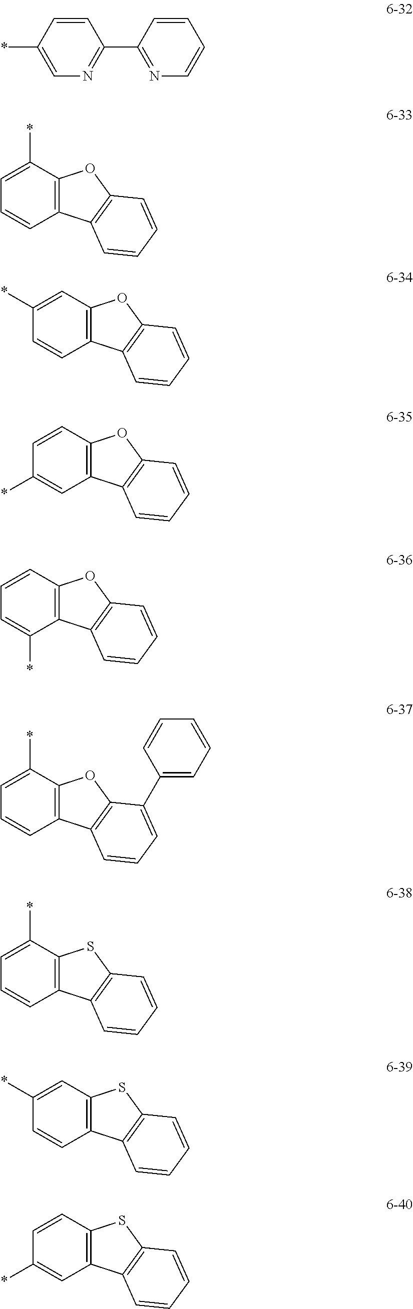 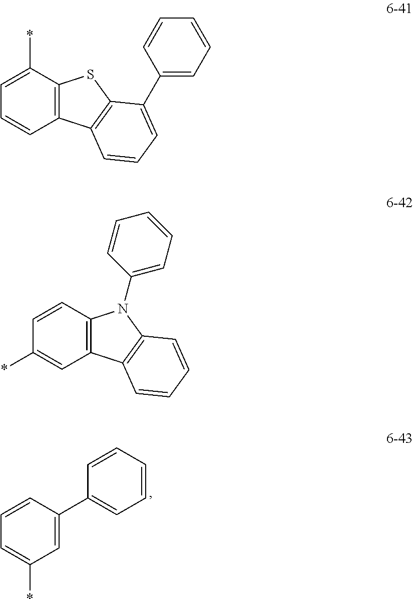
wherein, * in Formulae 6-1 to 6-43 indicates a binding site to a neighboring atom.

9. The condensed cyclic compound of claim 1, wherein R₁₁ to R₁₄ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), and N(Q₆)(Q₇).

10. The condensed cyclic compound of claim 1, wherein R₁₁ to R₁₄ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, —Si(Q₁)(Q₂)(Q₃), and groups represented by Formulae 7-1 to 7-18, wherein Q₁ to Q₃ are each independently selected from a C₁-C₁₀ alkyl group, a C₁-C₁₀ alkoxy group, a phenyl group, and a naphthyl group: 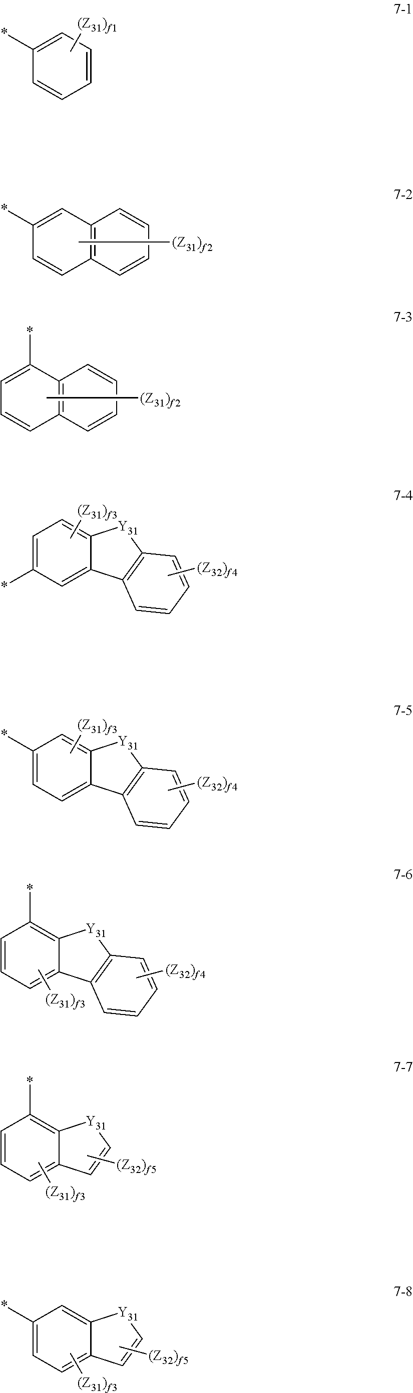 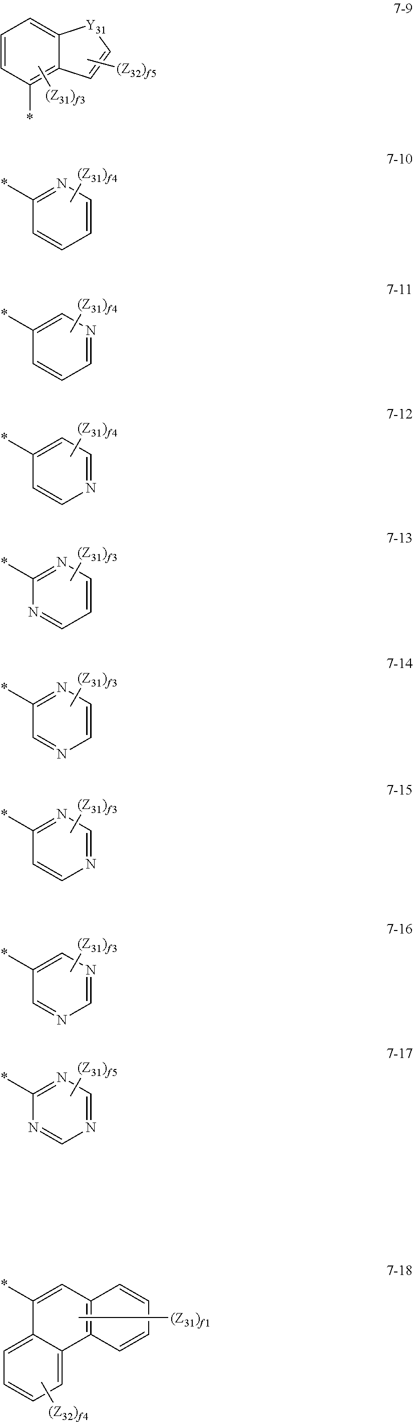
wherein, in Formulae 7-1 to 7-18,
Y₃₁ is selected from O, S, C(Z₃₃)(Z₃₄), N(Z₃₅), and Si(Z₃₆)(Z₃₇),
Z₃₁ to Z₃₇ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,
f1 is an integer selected from 1, 2, 3, 4, and 5, f2 is an integer selected from 1, 2, 3, 4, 5, 6, and 7, f3 is an integer selected from 1, 2, and 3, f4 is an integer selected from 1, 2, 3, and 4, f5 is 1 or 2, and
* indicates a binding site to a neighboring atom.

11. The condensed cyclic compound of claim 1, wherein R₁₁ to R₁₄ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, —Si(Q₁)(Q₂)(Q₃), and groups represented by Formulae 8-1 to 8-29, wherein Q₁ to Q₃ are each independently selected from a C₁-C₁₀ alkyl group, a C₁-C₁₀ alkoxy group, a phenyl group, and a naphthyl group: 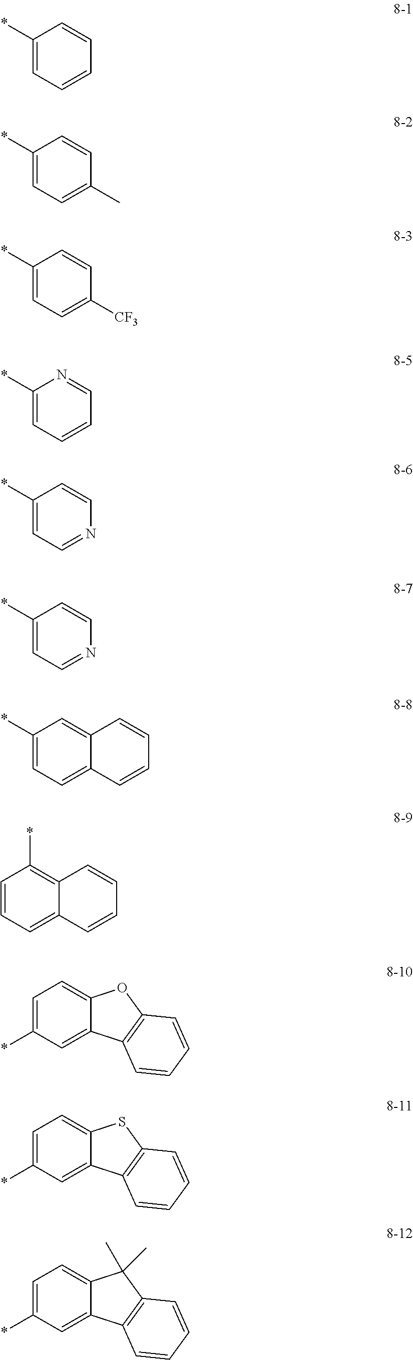 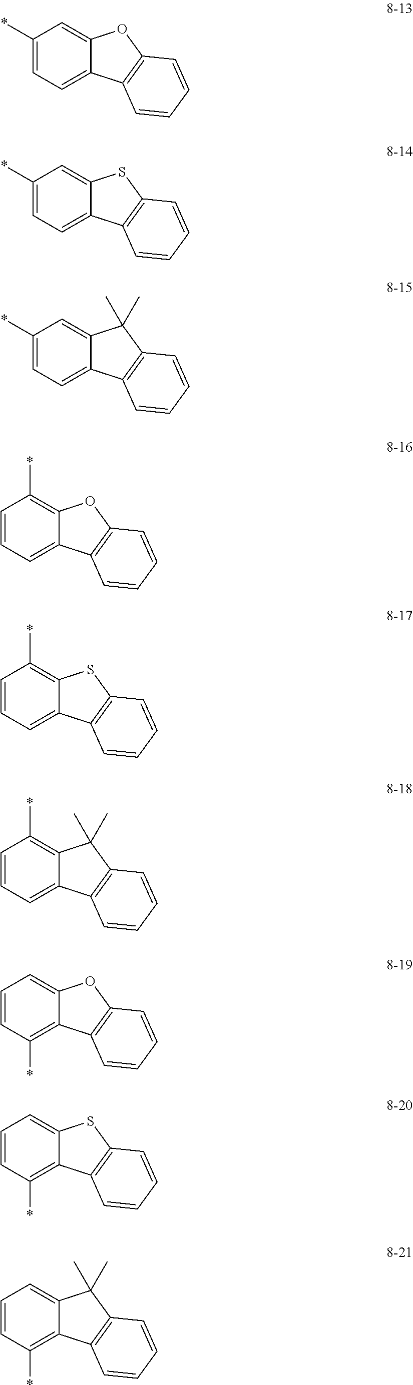 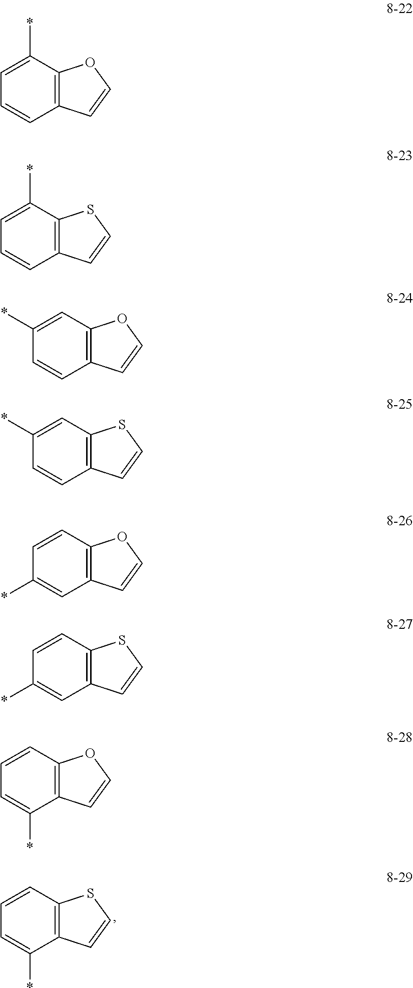
wherein, * in Formulae 8-1 to 8-29 indicates a binding site to a neighboring atom.

12. The condensed cyclic compound of claim 1, wherein the condensed cyclic compound is represented by any one of Formulae 1A to 1I: 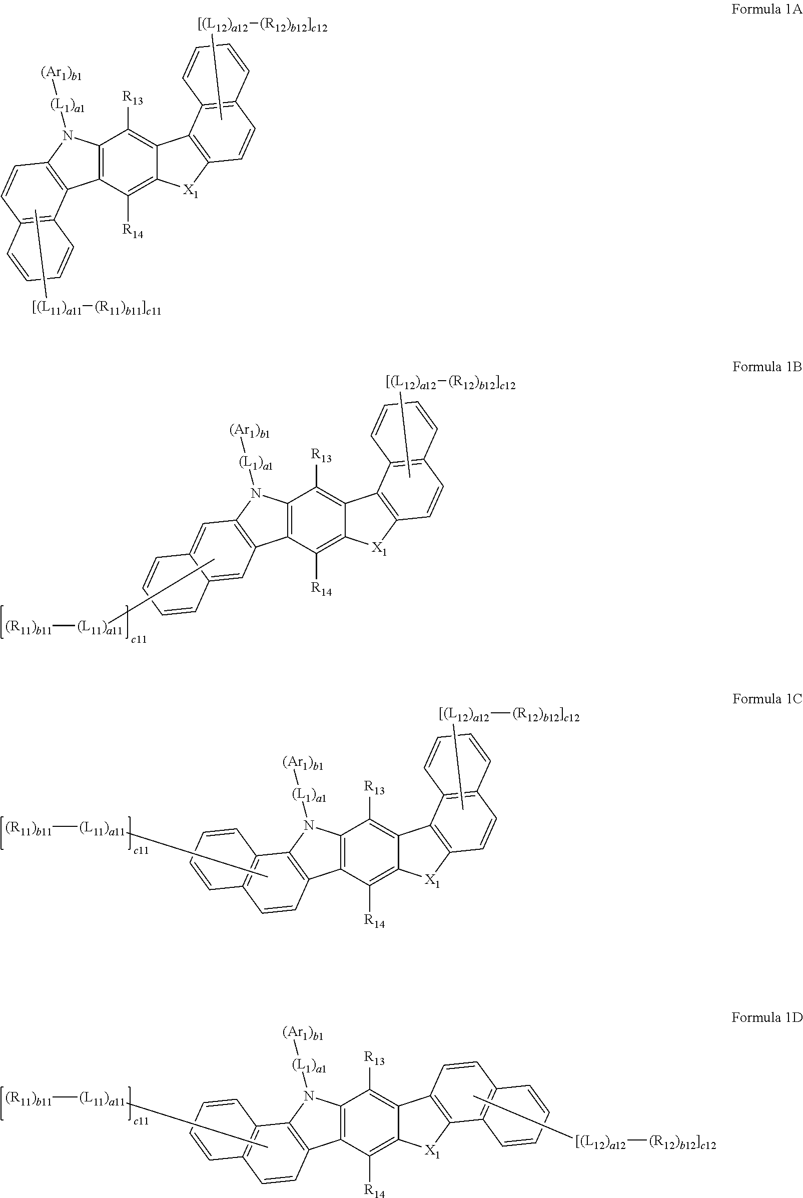 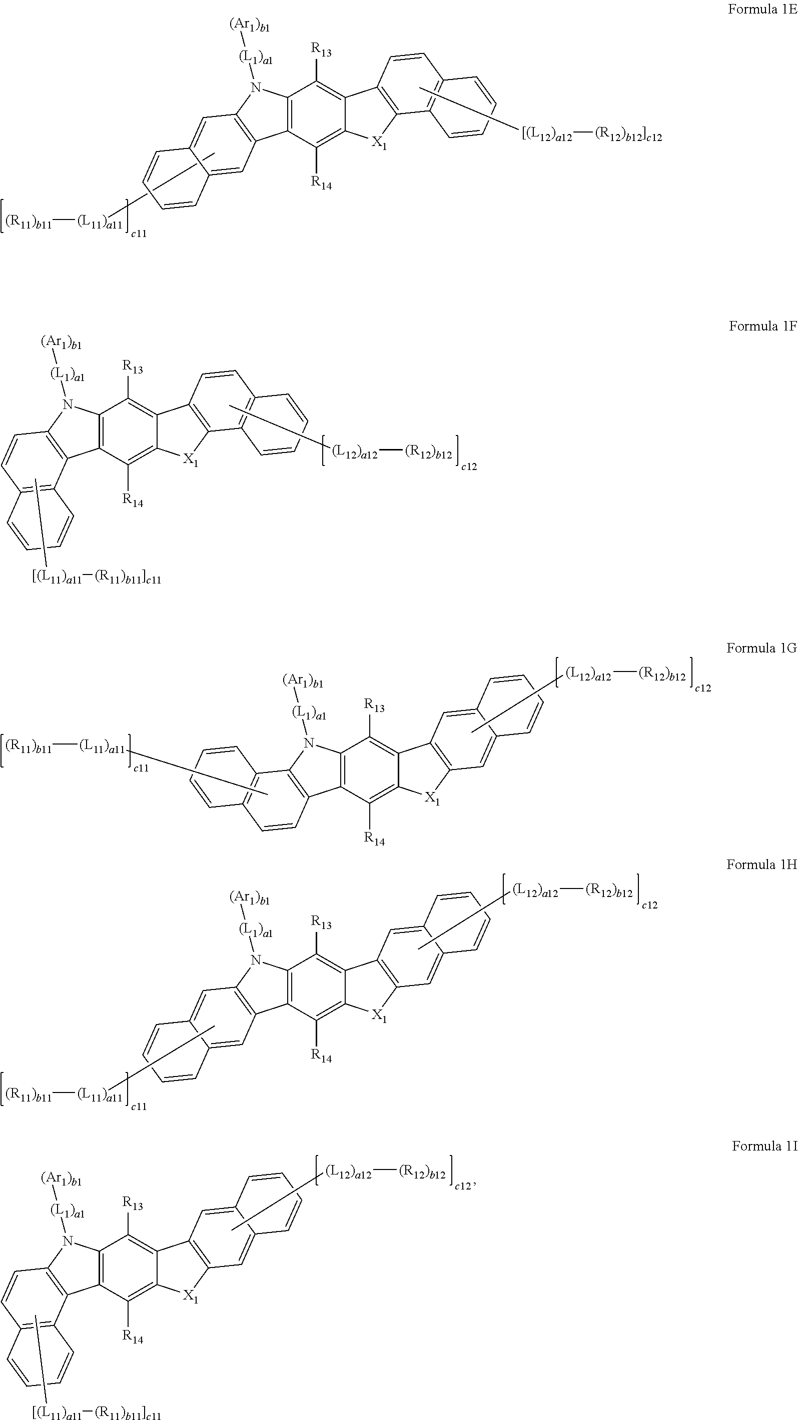
wherein, descriptions of X₁, L₁, L₁₁, L₁₂, a1, a11, a12, Ar₁, b1, R₁₁ to R₁₄, b11, b12, c11, and c12 in Formulae 1A to 1I are respectively the same as in Formula 1.

13. The condensed cyclic compound of claim 1, wherein the condensed cyclic compound is represented by any one of Formulae 2A to 2I: 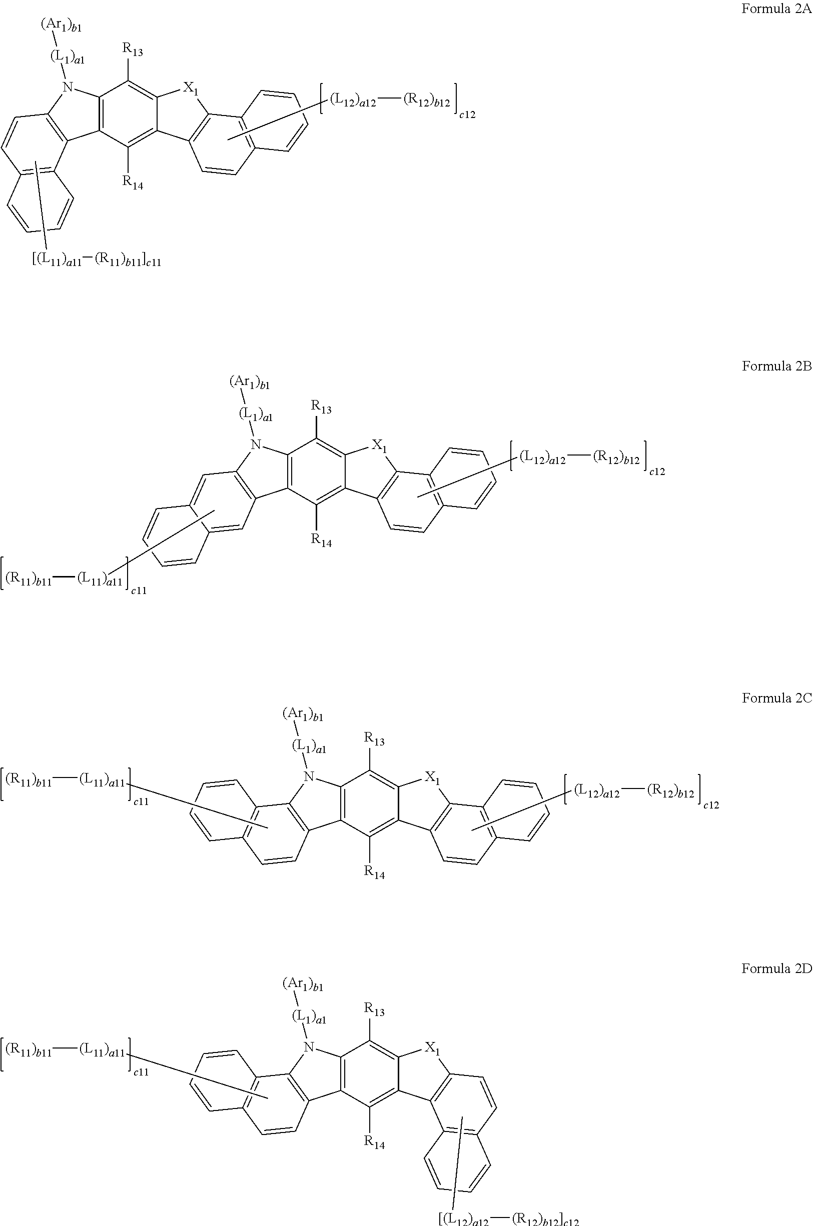 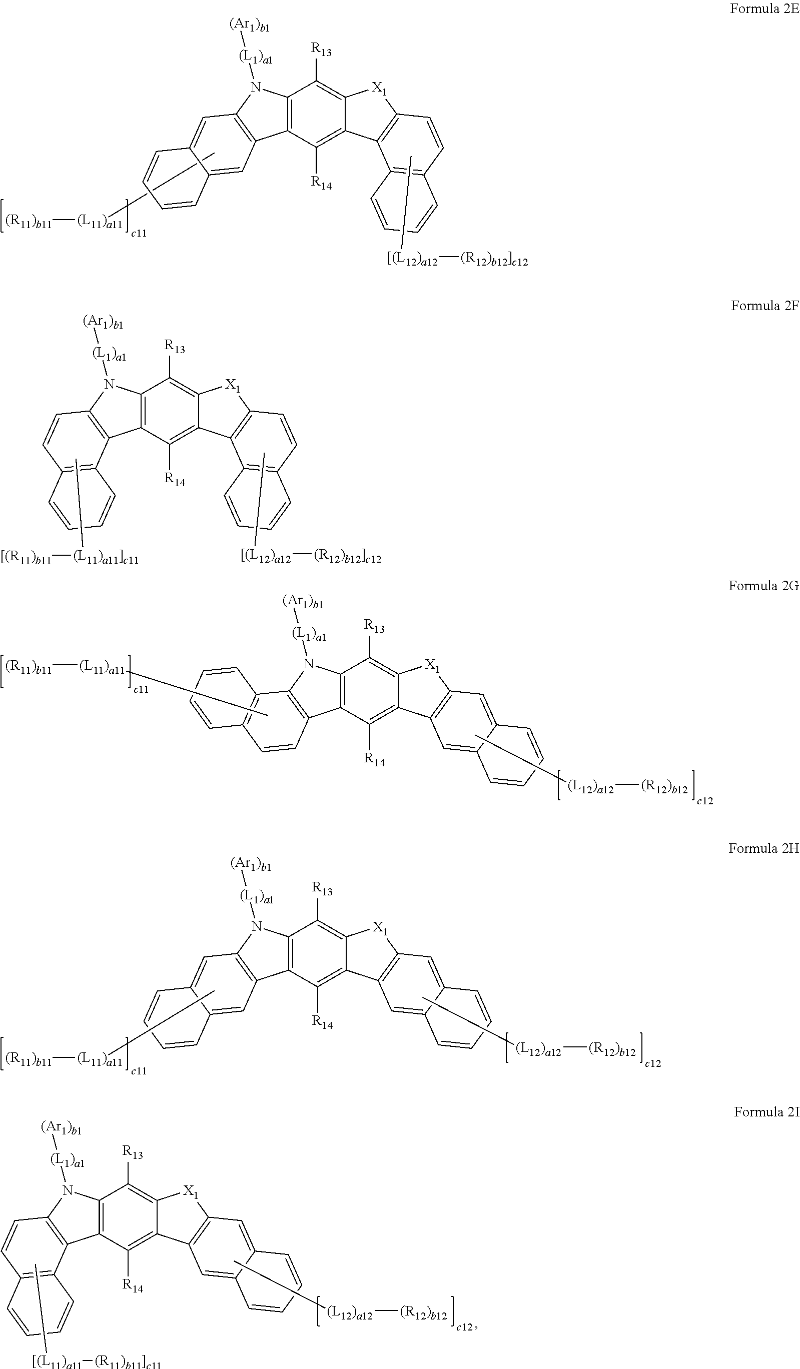
wherein, descriptions of X₁, L₁, L₁₁, L₁₂, a1, a11, a12, Ar₁, b1, R₁₁ to R₁₄, b11, b12, c11, and c12 in Formulae 2A to 2I are respectively the same as in Formula 1.

14. The condensed cyclic compound of claim 1, wherein the condensed cyclic compound is represented by any one of Formulae 1A-1 to 1A-4 and 2F-1 to 2F-4: 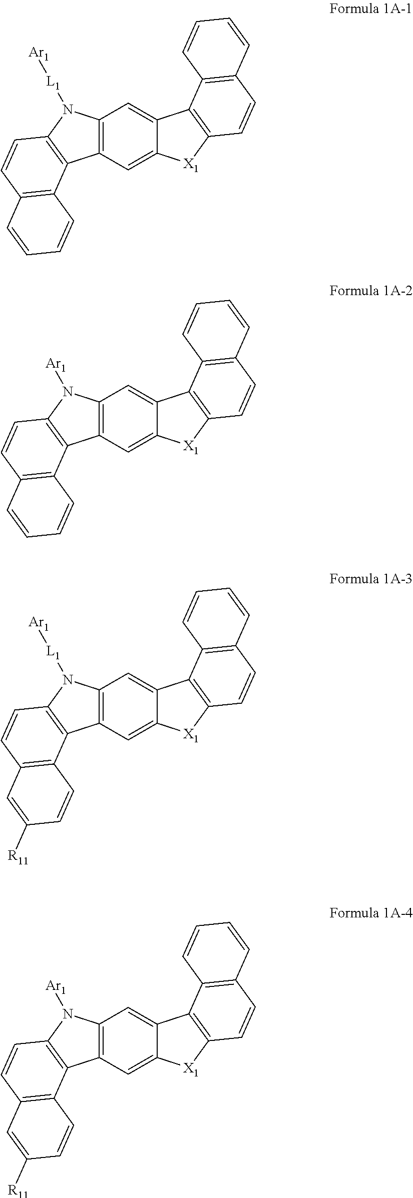 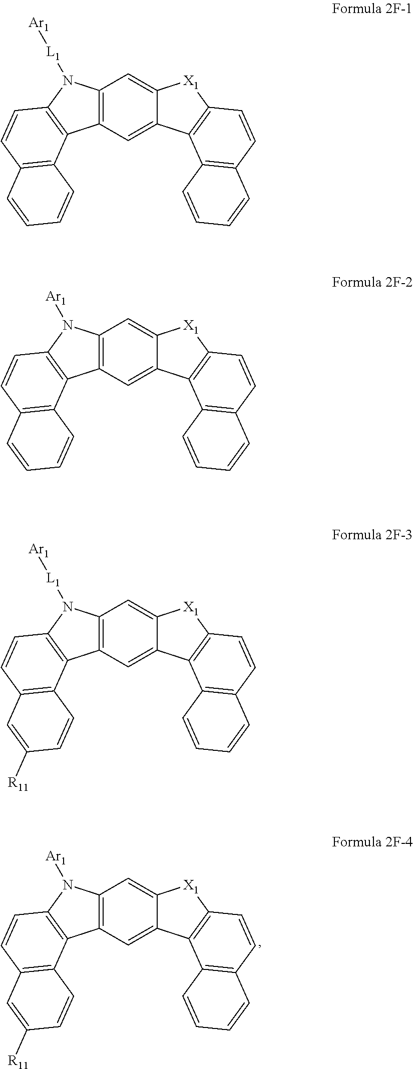
wherein, in Formulae 1A-1 to 1A-4 and 2F-1 to 2F-4,
X₁ is O or S,
L₁ is selected from groups represented by Formulae 4-1 to 4-25,
Ar₁ is selected from groups represented by Formulae 6-1 to 6-43, and
R₁₁ is selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, 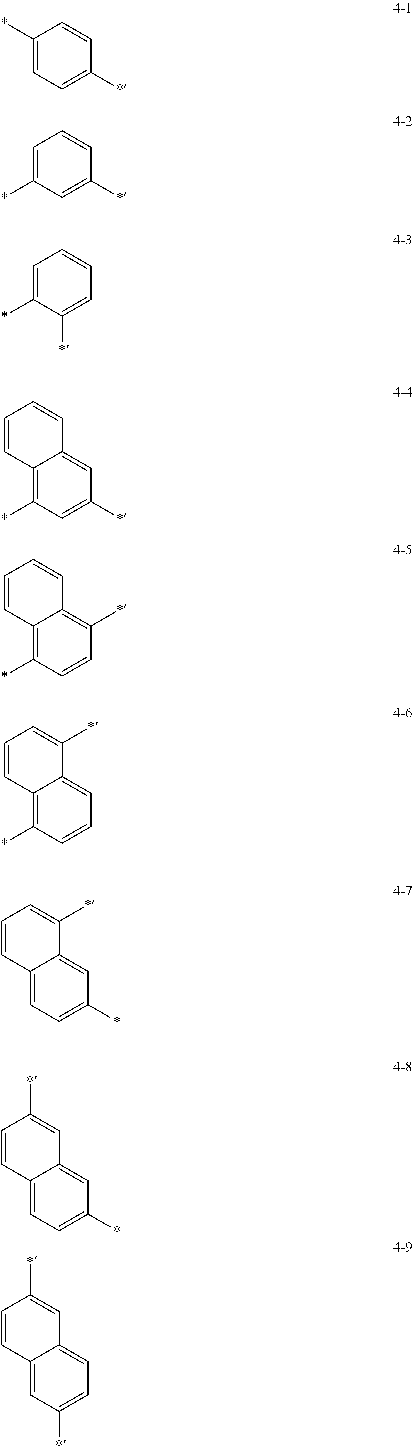 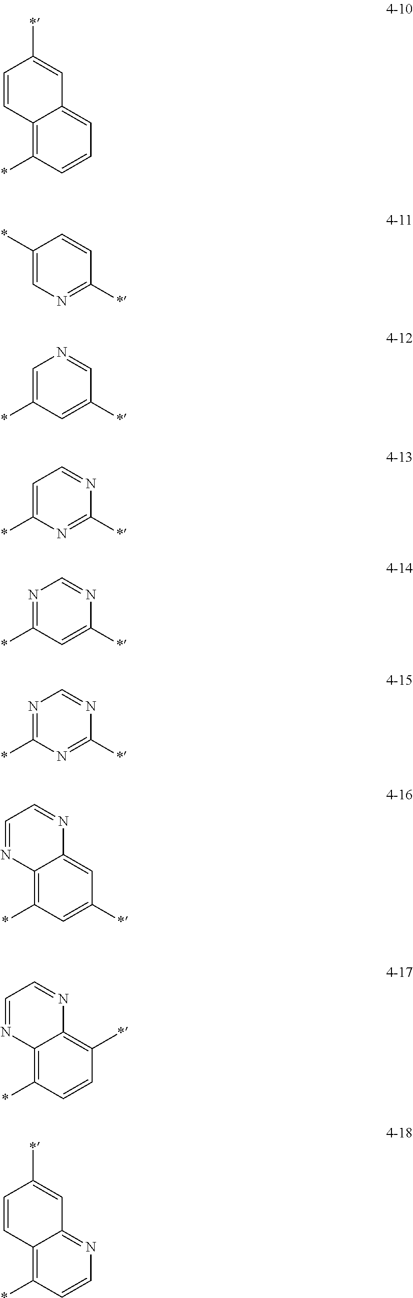 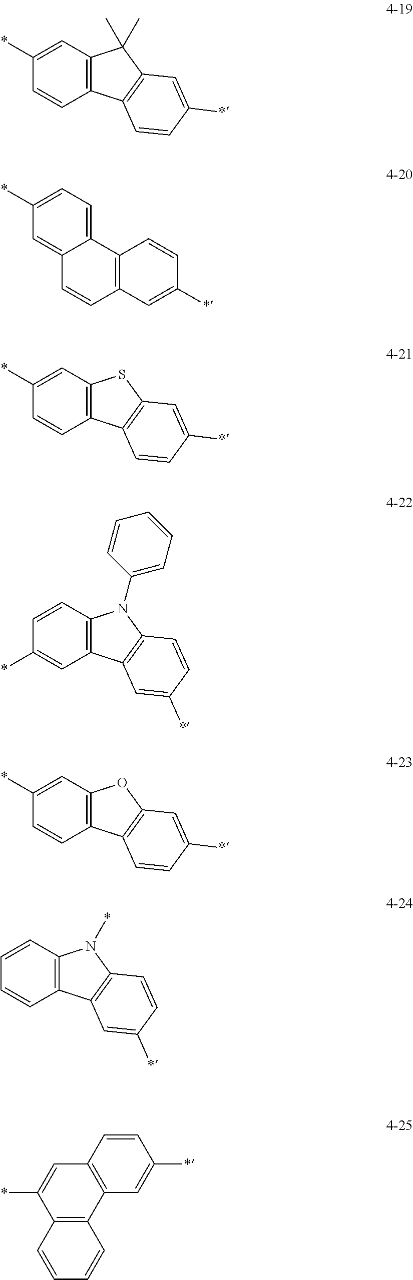 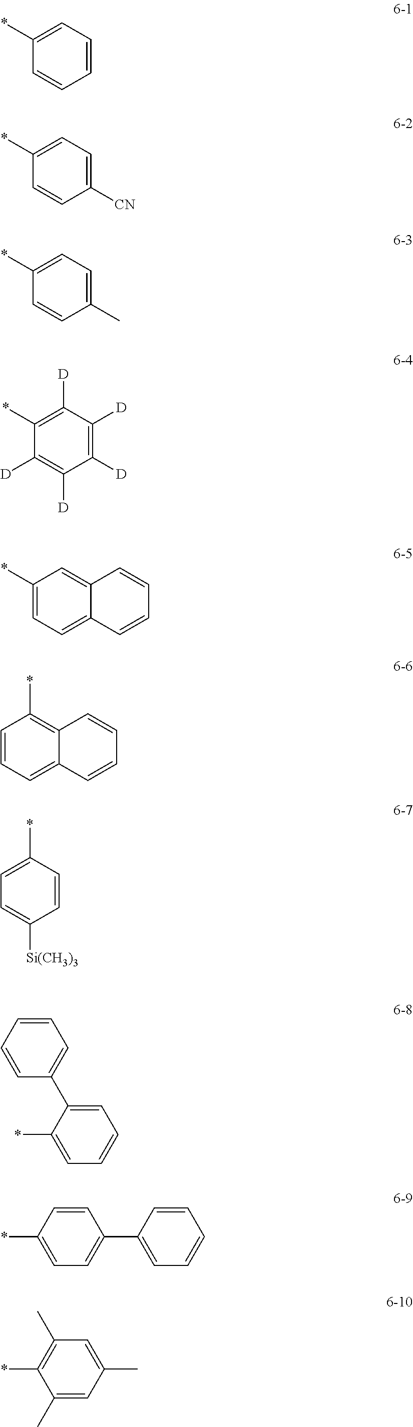 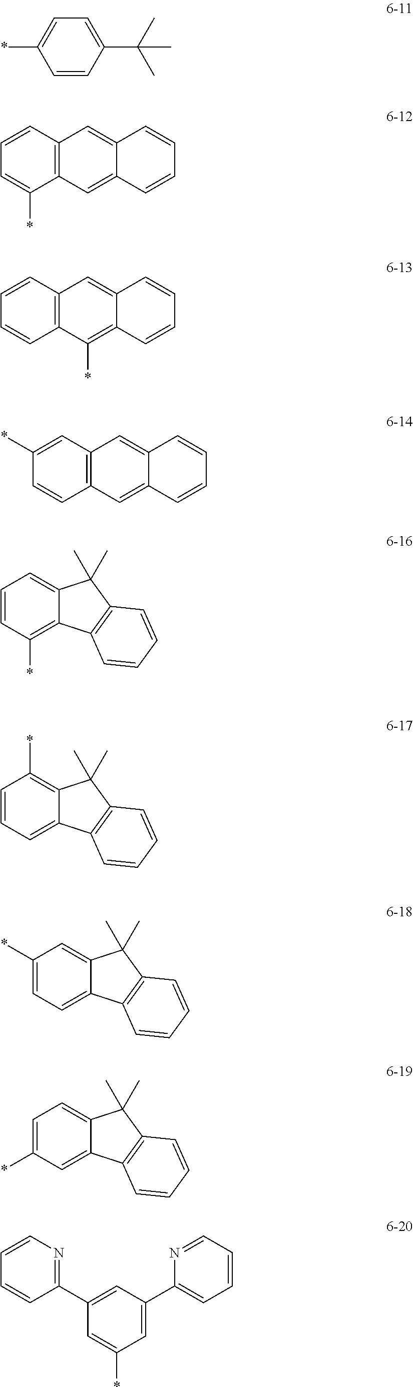 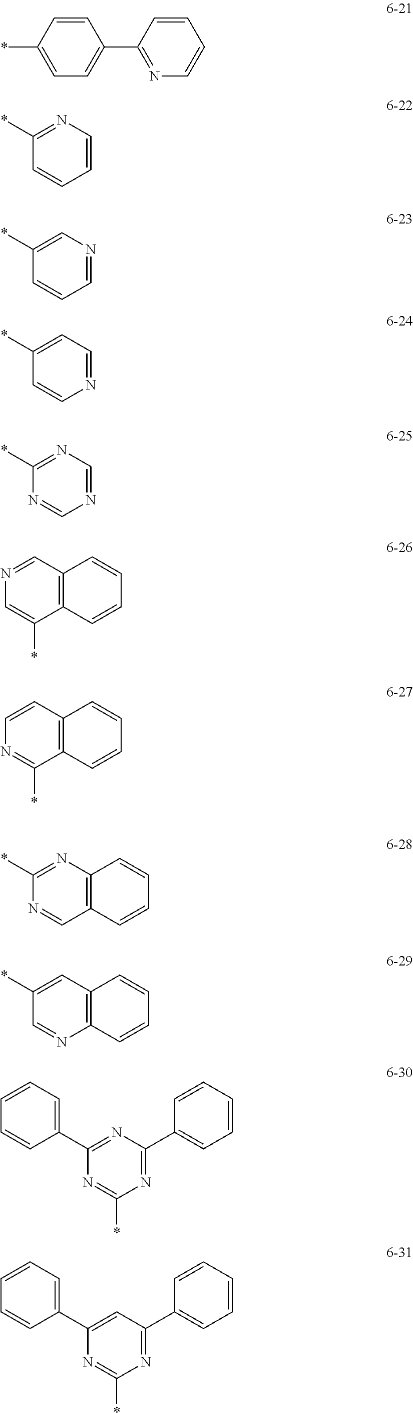 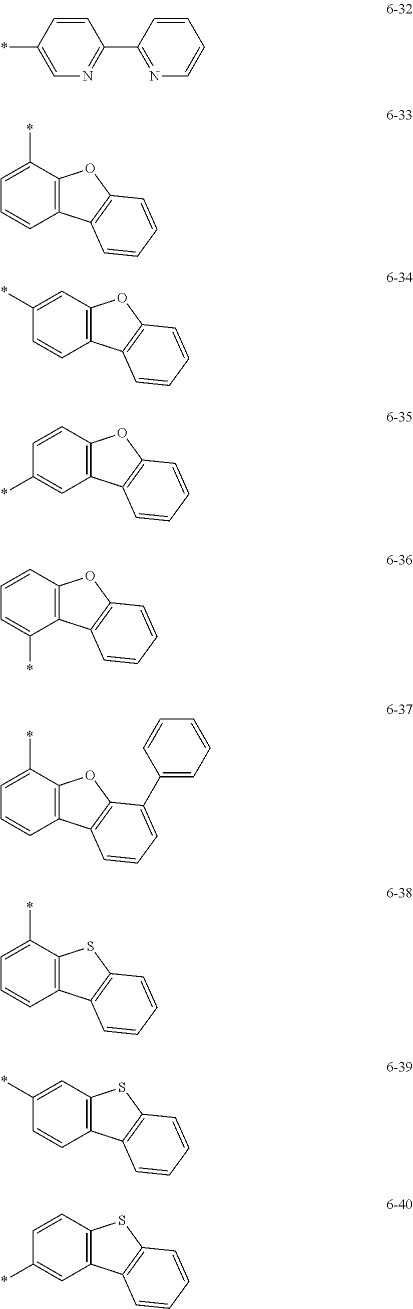 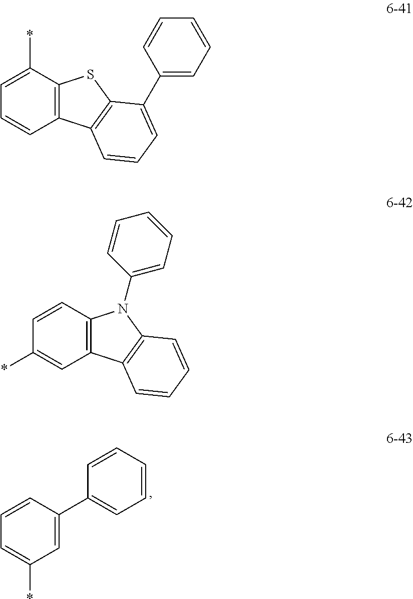 [SMILES: *c1cccc2c1sc1c(-c3ccccc3)cccc12 ; *c1ccc2c(c1)c1ccccc1n2-c1ccccc1 ; *c1cccc(-c2ccccc2)c1]
wherein * in Formulae 4-1 to 4-25 and Formulae 6-1 to 6-43 indicates a binding site to a neighboring atom.

15. The condensed cyclic compound of claim 1, wherein the condensed cyclic compound is one of Compounds 1 to 32:

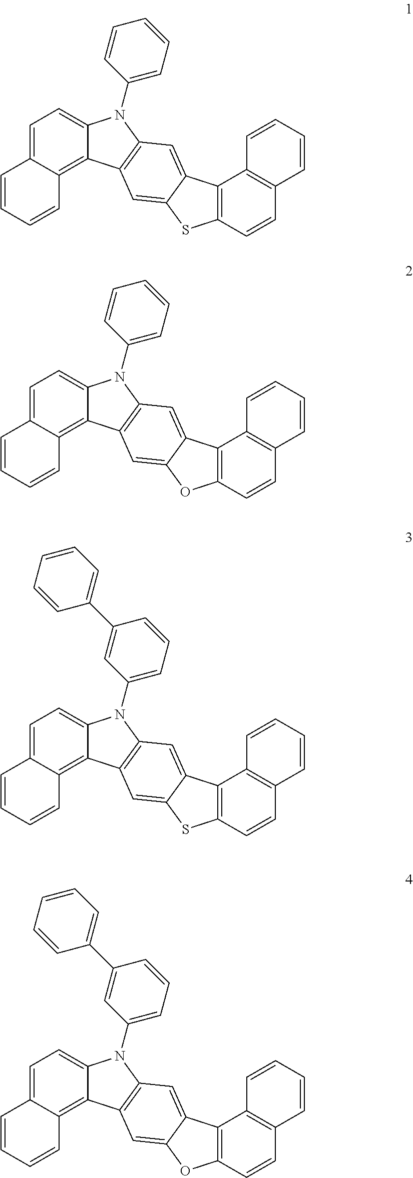 [SMILES: c1ccc(-n2c3cc4c(cc3c3c5ccccc5ccc32)sc2ccc3ccccc3c24)cc1 ; c1ccc(-n2c3cc4c(cc3c3c5ccccc5ccc32)oc2ccc3ccccc3c24)cc1 ; c1ccc(-c2cccc(-n3c4cc5c(cc4c4c6ccccc6ccc43)sc3ccc4ccccc4c35)c2)cc1 ; c1ccc(-c2cccc(-n3c4cc5c(cc4c4c6ccccc6ccc43)oc3ccc4ccccc4c35)c2)cc1]

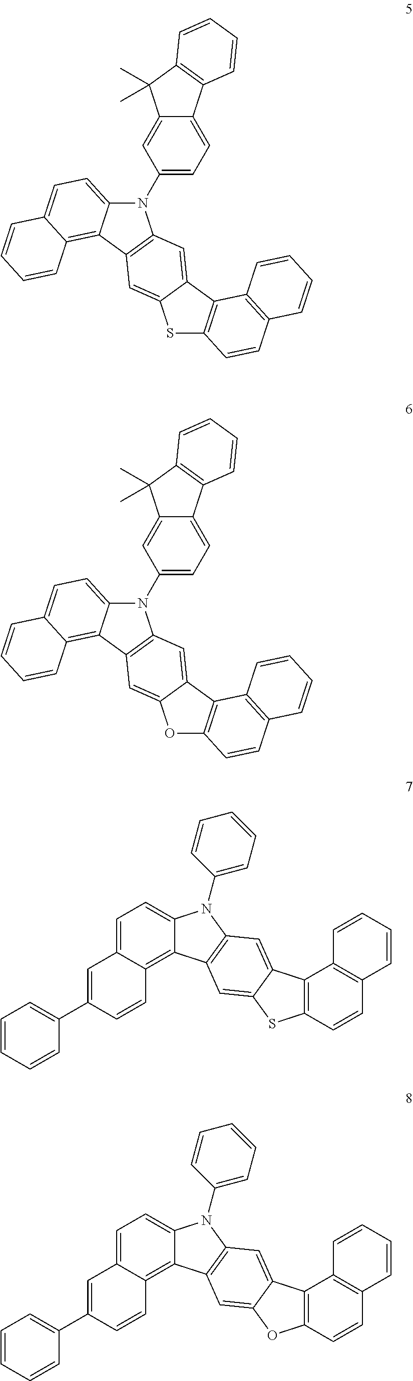 [SMILES: CC1(C)c2ccccc2-c2ccc(-n3c4cc5c(cc4c4c6ccccc6ccc43)sc3ccc4ccccc4c35)cc21 ; CC1(C)c2ccccc2-c2ccc(-n3c4cc5c(cc4c4c6ccccc6ccc43)oc3ccc4ccccc4c35)cc21 ; c1ccc(-c2ccc3c(ccc4c3c3cc5sc6ccc7ccccc7c6c5cc3n4-c3ccccc3)c2)cc1 ; c1ccc(-c2ccc3c(ccc4c3c3cc5oc6ccc7ccccc7c6c5cc3n4-c3ccccc3)c2)cc1]

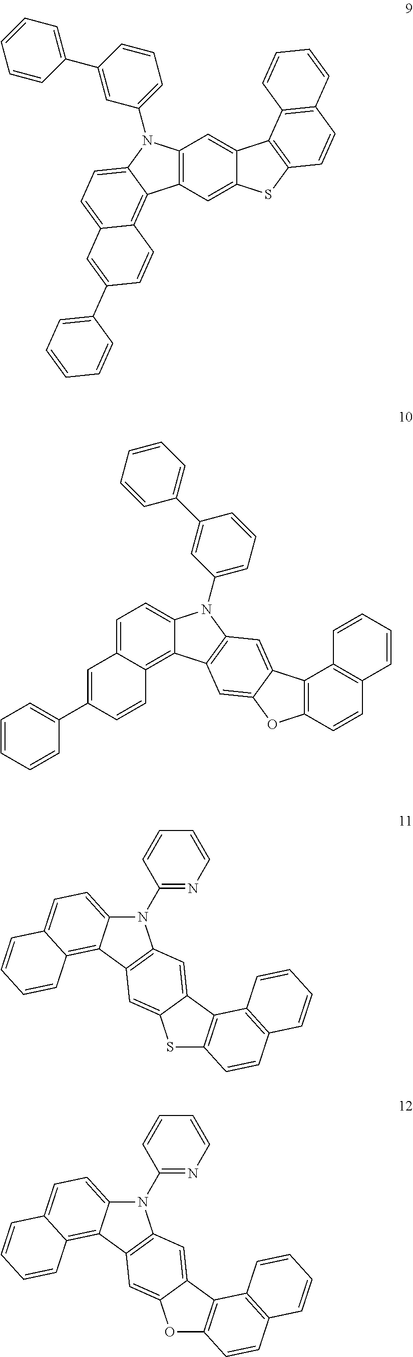 [SMILES: c1ccc(-c2cccc(-n3c4cc5c(cc4c4c6ccc(-c7ccccc7)cc6ccc43)sc3ccc4ccccc4c35)c2)cc1 ; c1ccc(-c2cccc(-n3c4cc5c(cc4c4c6ccc(-c7ccccc7)cc6ccc43)oc3ccc4ccccc4c35)c2)cc1 ; c1ccc(-n2c3cc4c(cc3c3c5ccccc5ccc32)sc2ccc3ccccc3c24)nc1 ; c1ccc(-n2c3cc4c(cc3c3c5ccccc5ccc32)oc2ccc3ccccc3c24)nc1]

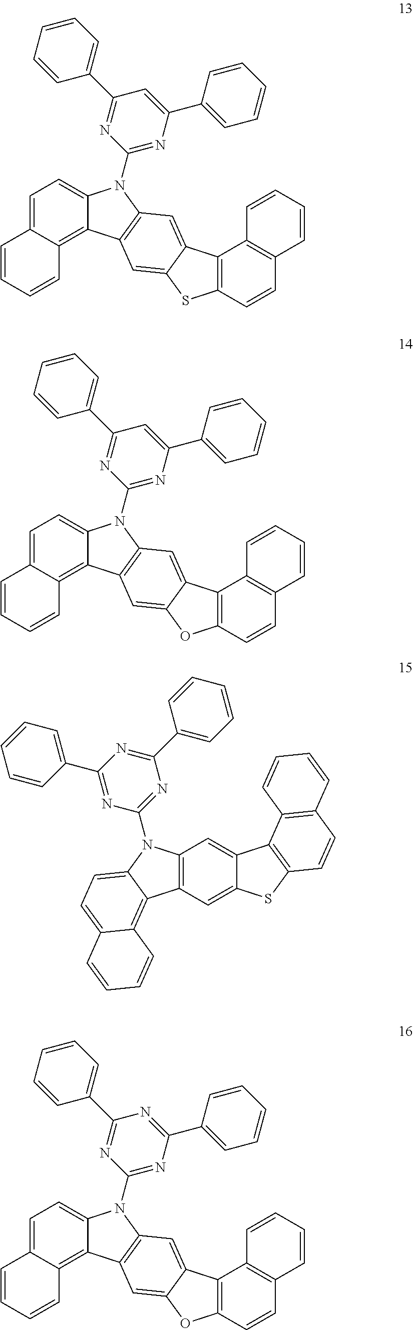

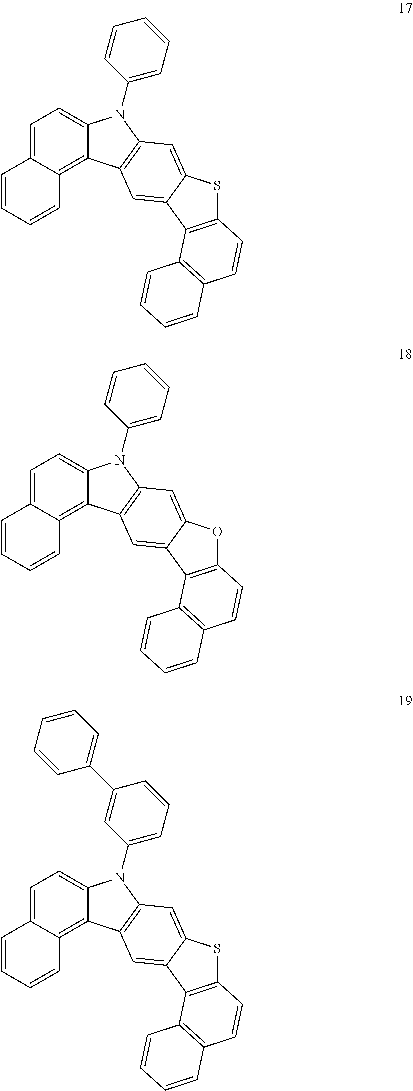

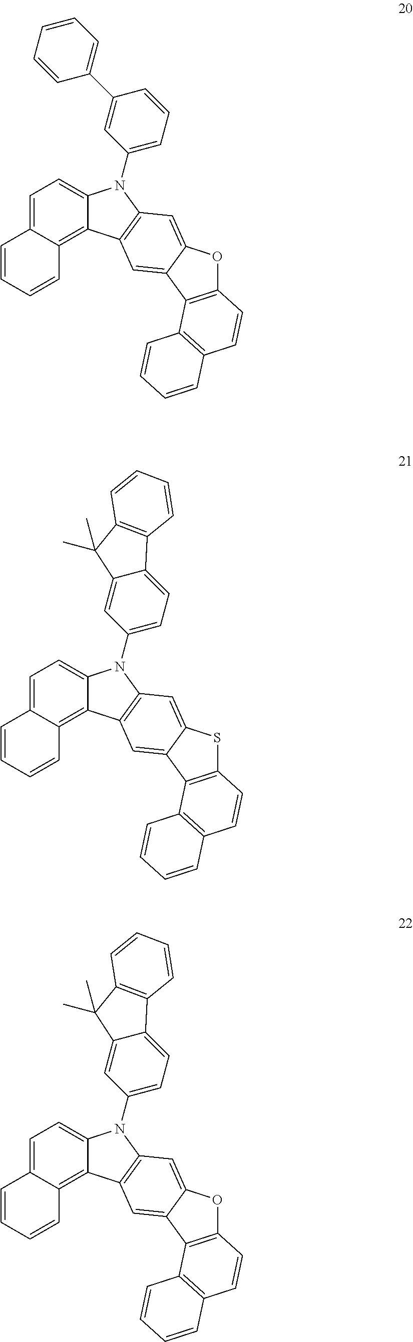 [SMILES: c1ccc(-c2cccc(-n3c4cc5oc6ccc7ccccc7c6c5cc4c4c5ccccc5ccc43)c2)cc1 ; CC1(C)c2ccccc2-c2ccc(-n3c4cc5sc6ccc7ccccc7c6c5cc4c4c5ccccc5ccc43)cc21 ; CC1(C)c2ccccc2-c2ccc(-n3c4cc5oc6ccc7ccccc7c6c5cc4c4c5ccccc5ccc43)cc21]

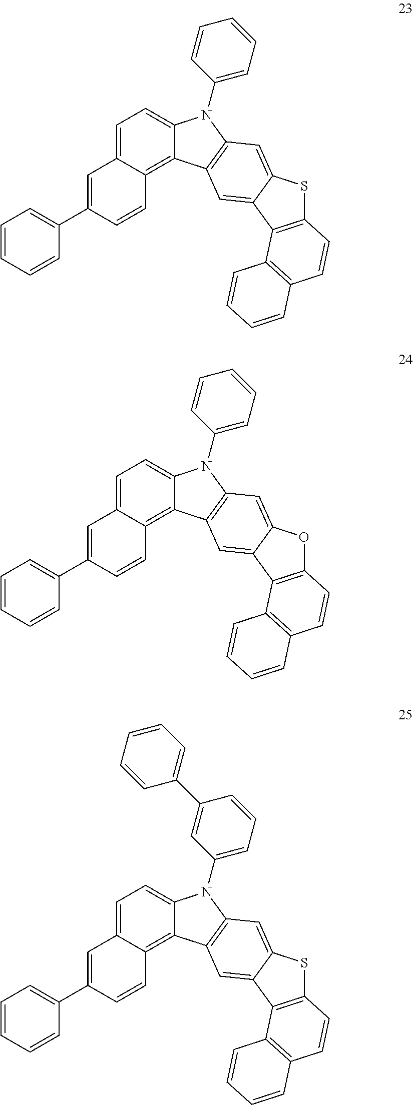

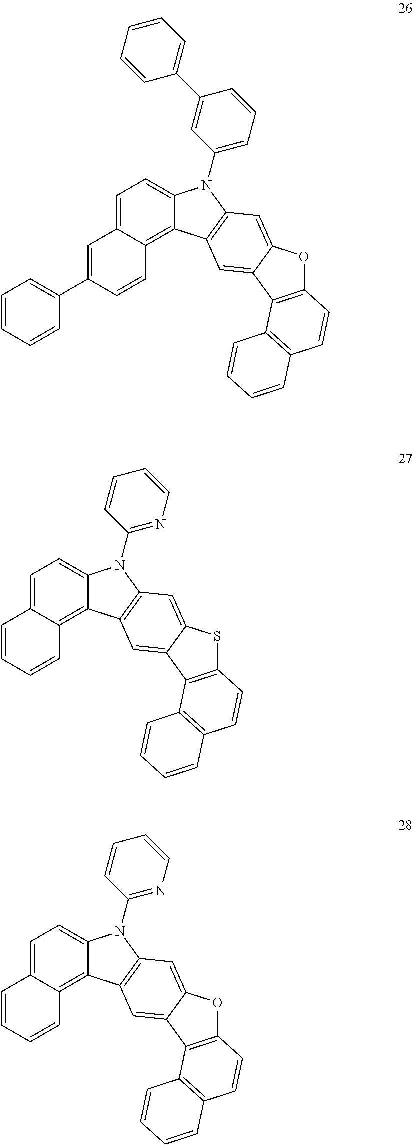 [SMILES: c1ccc(-c2cccc(-n3c4cc5oc6ccc7ccccc7c6c5cc4c4c5ccc(-c6ccccc6)cc5ccc43)c2)cc1 ; c1ccc(-n2c3cc4sc5ccc6ccccc6c5c4cc3c3c4ccccc4ccc32)nc1 ; c1ccc(-n2c3cc4oc5ccc6ccccc6c5c4cc3c3c4ccccc4ccc32)nc1]

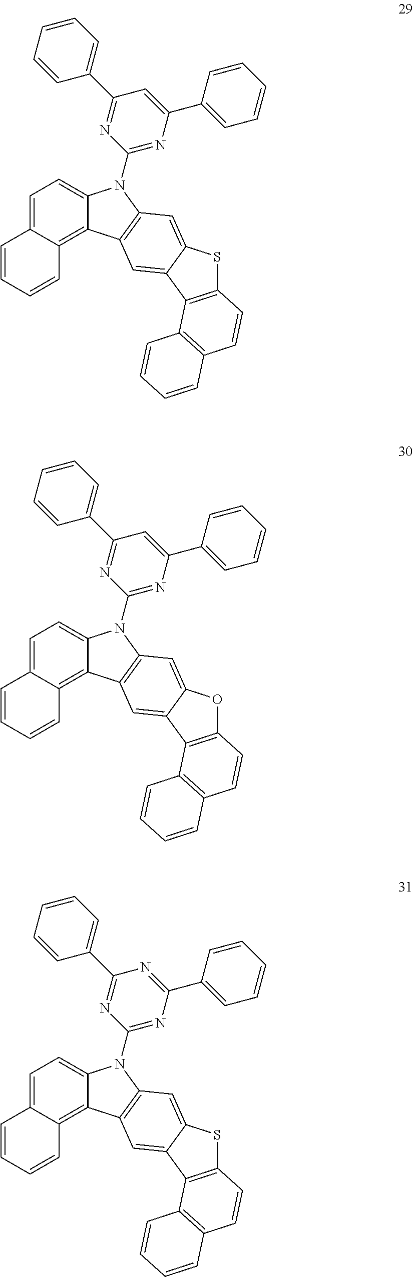 [SMILES: c1ccc(-c2cc(-c3ccccc3)nc(-n3c4cc5sc6ccc7ccccc7c6c5cc4c4c5ccccc5ccc43)n2)cc1 ; c1ccc(-c2cc(-c3ccccc3)nc(-n3c4cc5oc6ccc7ccccc7c6c5cc4c4c5ccccc5ccc43)n2)cc1 ; c1ccc(-c2nc(-c3ccccc3)nc(-n3c4cc5sc6ccc7ccccc7c6c5cc4c4c5ccccc5ccc43)n2)cc1]

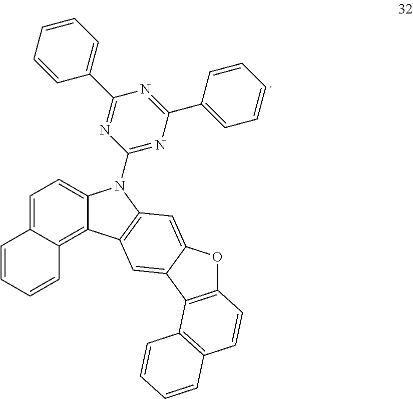 [SMILES: c1ccc(-c2nc(-c3ccccc3)nc(-n3c4cc5oc6ccc7ccccc7c6c5cc4c4c5ccccc5ccc43)n2)cc1]

16. An organic light-emitting device comprising: a first electrode; a second electrode facing the first electrode; and an organic layer between the first electrode and the second electrode, the organic layer comprising an emission layer, wherein the organic layer comprises at least one condensed cyclic compound of claim 1.

17. The organic light-emitting device of claim 16, wherein
the first electrode is an anode,
the second electrode is a cathode, and
the organic layer comprises:
a hole transport region between the first electrode and the emission layer, the hole transport region comprising at least one selected from a hole injection layer, a hole transport layer, a buffer layer, and an electron blocking layer, and
an electron transport region between the emission layer and the second electrode, the electron transport region comprising at least one selected from a hole blocking layer, an electron transport layer, and an electron injection layer.

18. The organic light-emitting device of claim 16, wherein the condensed cyclic compound is comprised in the emission layer.

19. The organic light-emitting device of claim 18, wherein the emission layer further comprises an organometallic complex represented by Formula 401: 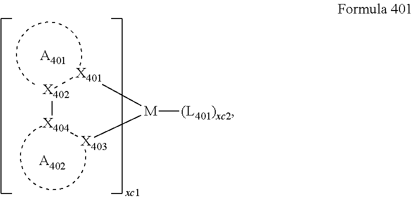
wherein, in Formula 401,
M is selected from iridium (Ir), platinum (Pt), osmium (Os), titanium (Ti), zirconium (Zr), hafnium (Hf), europium (Eu), terbium (Tb), and thulium (Tm),
X₄₀₁ to X₄₀₄ are each independently a nitrogen or a carbon,
A₄₀₁ ring and A₄₀₂ ring are each independently selected from a substituted or unsubstituted benzene, a substituted or unsubstituted naphthalene, a substituted or unsubstituted fluorene, a substituted or unsubstituted spiro-fluorene, a substituted or unsubstituted indene, a substituted or unsubstituted pyrrole, a substituted or unsubstituted thiophene, a substituted or unsubstituted furan, a substituted or unsubstituted imidazole, a substituted or unsubstituted pyrazole, a substituted or unsubstituted thiazole, a substituted or unsubstituted isothiazole, a substituted or unsubstituted oxazole, a substituted or unsubstituted isooxazole, a substituted or unsubstituted pyridine, a substituted or unsubstituted pyrazine, a substituted or unsubstituted pyrimidine, a substituted or unsubstituted pyridazine, a substituted or unsubstituted quinoline, a substituted or unsubstituted isoquinoline, a substituted or unsubstituted benzoquinoline, a substituted or unsubstituted quinoxaline, a substituted or unsubstituted quinazoline, a substituted or unsubstituted carbazole, a substituted or unsubstituted benzoimidazole, a substituted or unsubstituted benzofuran, a substituted or unsubstituted benzothiophene, a substituted or unsubstituted isobenzothiophene, a substituted or unsubstituted benzoxazole, a substituted or unsubstituted isobenzoxazole, a substituted or unsubstituted triazole, a substituted or unsubstituted oxadiazole, a substituted or unsubstituted triazine, a substituted or unsubstituted dibenzofuran, and a substituted or unsubstituted dibenzothiophene,
at least one substituent of the substituted benzene, substituted naphthalene, substituted fluorene, substituted spiro-fluorene, substituted indene, substituted pyrrole, substituted thiophene, substituted furan, substituted imidazole, substituted pyrazole, substituted thiazole, substituted isothiazole, substituted oxazole, substituted isoxazole, substituted pyridine, substituted pyrazine, substituted pyrimidine, substituted pyridazine, substituted quinoline, substituted isoquinoline, substituted benzoquinoline, substituted quinoxaline, substituted quinazoline, substituted carbazole, substituted benzoimidazole, substituted benzofuran, substituted benzothiophene, substituted isobenzothiophene, substituted benzoxazole, substituted isobenzoxazole, substituted triazole, substituted oxadiazole, substituted triazine, substituted dibenzofuran, and substituted dibenzothiophene is selected from:
deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₄₀₁)(Q₄₀₂), —Si(Q₄₀₃)(Q₄₀₄)(Q₄₀₅), and —B(Q₄₀₆)(Q₄₀₇);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₄₁₁)(Q₄₁₂), —Si(Q₄₁₃)(Q₄₁₄)(Q₄₁₅), and —B(Q₄₁₆)(Q₄₁₇); and
—N(Q₄₂₁)(Q₄₂₂), —Si(Q₄₂₃)(Q₄₂₄)(Q₄₂₅), and —B(Q₄₂₆)(Q₄₂₇),
L₄₀₁ is an organic ligand,
xc1 is 1, 2, or 3, and
xc2 is 0, 1, 2, or 3,
wherein Q₄₀₁ to Q₄₀₇, Q₄₁₁ to Q₄₁₇, and Q₄₂₁ to Q₄₂₇ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

20. The organic light-emitting device of claim 19, wherein an amount of the condensed cyclic compound in the emission layer is greater than that of the organometallic complex in the emission layer.
